(19) United States
(12) Patent Application Publication
(10) Pub. No.: US 2023/0398192 A1
(43) Pub. Date: Dec. 14, 2023

(54) METHODS OF TREATING METACHROMATIC LEUKODYSTROPHY

(71) Applicant: Genzyme Corporation, Cambridge, MA (US)

(72) Inventor: Shyam RAMACHANDRAN, Winchester, MA (US)

(21) Appl. No.: 18/317,772

(22) Filed: May 15, 2023

Related U.S. Application Data
(60) Provisional application No. 63/459,564, filed on Apr. 14, 2023.
(60) Provisional application No. 63/342,590, filed on May 16, 2022.

(51) Int. Cl. A61K 38/46; C12N 15/86; C12N 9/16; A61K 48/00; A61P 25/28

(52) U.S. Cl. CPC A61K 38/465; C12N 15/86; C12N 9/16; C12Y 301/06001; A61K 48/0058; A61K 48/0075; A61P 25/28; C12N 2750/14143; C12N 2750/14145; C12N 2750/14171

(57) ABSTRACT

Provided herein are expression cassettes for expressing a transgene in a liver cell, wherein the transgene encodes an ARSA polypeptide. Also provided are methods to treat metachromatic leukodystrophy (MLD). Further provided herein are vectors (e.g., rAAV vectors), viral particles, pharmaceutical compositions and kits for expressing an ARSA polypeptide in an individual in need thereof.

### CROSS-REFERENCE TO RELATED APPLICATIONS

This application claims priority to U.S. Provisional Application No. 63/342,590, filed May 16, 2022 and U.S. Provisional Application No. 63/459,564, filed Apr. 14, 2023, the contents of which are incorporated herein by reference in their entirety.

### REFERENCE TO AN ELECTRONIC SEQUENCE LISTING

The contents of the electronic sequence listing (159792018400SEQLIST.xml; Size: 36,314 bytes; and Date of Creation: May 8, 2023) is herein incorporated by reference in its entirety.

### FIELD OF THE INVENTION

The present disclosure relates to provides methods for treating metachromatic leukodystrophy (MLD) in a patient in need thereof, comprising administering to the cerebrospinal fluid (CSF) of a patient a recombinant adeno-associated virus (rAAV) viral particle comprising a vector encoding arylsulfatase A (ARSA).

### BACKGROUND

Metachromatic leukodystrophy (MLD) is an autosomal recessive neurodegenerative disorder caused by mutations in the enzyme arylsulfatase A (ARSA). Reduced levels of ARSA activity result in toxic accumulation of sulfatide characterized by degeneration of myelin-forming cells (oligodendrocytes and Schwann cells) in the central and peripheral nervous system. This results in demyelination, dysfunction, degeneration of neurons and neuroinflammation (astrocytosis, microglial activation). Clinical manifestations are primarily in the nervous system, resulting in intellectual disability, emotional and behavioral problems, loss of motor skills (moving, speaking, swallowing), poor muscle function and paralysis, blindness, hearing loss and seizures.

The current treatment for MILD involves autologous hematopoietic stem cell transplantation (HSCT) involving allogenic bone marrow transplant.

### BRIEF SUMMARY OF THE INVENTION

In some aspects, the invention provides methods for treating metachromatic leukodystrophy (MLD) in a patient in need thereof, comprising administering to the cerebrospinal fluid (CSF) of the patient a recombinant adeno-associated virus (rAAV) viral particle comprising a vector encoding the enzyme arylsulfatase A (ARSA) (also referred to herein as the ARSA polypeptide). In some embodiments, the viral particle is administered directly to the CSF via intracerebroventricular (ICV) administration, direct cisterna magna (dCM) administration or an intrathecal microcatheter (IT-CM).

The present invention is based at least in part on the development of rAAV viral particles comprising (a) a rAAV vector comprising an expression cassette that encodes an arylsulfatase A (ARSA) polypeptide and (b) a capsid capable of transducing the cells of the central nervous system (CNS). Administration of the viral particles into the CSF provides high levels of expression of the ARSA protein in the brain. In particular embodiments, the expression cassette of the viral particle is able to drive transgene expression in the central and peripheral nervous systems to treat MLD. In some embodiments, the expression cassette comprises a ARSA gene of SEQ ID NO: 2. In some embodiments, the ARSA gene expresses an ARSA polypeptide having a SEQ ID NO:1.

In some aspects, the invention provides a recombinant adeno-associated virus (rAAV) particle comprising a rAAV vector, wherein the rAAV vector comprises an expression cassette for expressing an ARSA polypeptide in the central and peripheral nervous systems, wherein the expression cassette comprises a transgene operably linked to a promoter and optionally an enhancer, wherein the transgene encodes an ARSA polypeptide, and wherein the AAV viral particle comprises a capsid protein capable of transducing the cells of the central nervous system (CNS). In some embodiments, the AAV capsid protein is an AAV9 capsid protein (SEQ ID NO:9). In some embodiments, the capsid of the rAAV is a modified AAV9 capsid protein. In some embodiments, the capsid of the rAAV is AAV.rh10 capsid protein. In some embodiments, the ARSA gene expresses an ARSA polypeptide having a SEQ ID NO:1.

In some embodiments, the disclosure provides rAAV particles for intra-CSF administration of an ARSA polypeptide that comprise a modified AAV9 capsid protein comprising a targeting peptide that targets the rAAV particles to the brain. Such modified AAV9 capsids are described in International Publication No. WO 2021/102234 A1, which is hereby incorporated by reference in its entirety. In particular embodiments, the targeting peptide of the modified AAV9 capsids are inserted after residue 588 of the AAV9 structural protein. In some embodiments, the targeting peptide has SEQ ID NO: 10. In some embodiments, the targeting peptide is flanked by linker sequences on the N-terminal and the C-terminal end of the targeting peptide. In some embodiments, the linker sequence on the N-terminal side has the sequence AAA. In some embodiments, the linker sequence on the C-terminal side is AS. In some embodiments, the full sequence inserted after residue 588 of the AAV9 capsid structural protein has SEQ ID NO: 11. In some embodiments, the full modified AAV9 capsid structural protein (VP1) has SEQ ID NO: 12. The capsid protein having SEQ ID NO: 12 is referred to herein as the AAV1999. In some embodiments, the full modified AAV9 capsid structural protein that it at least 90% (e.g., at least 92%, at least 95%, at least 98%, at least 98.5%, at least 99%, at least 99.2%, at least 99.5%, or at least 99.8%) identical to SEQ ID NO: 12, wherein the modified AAV9 structural capsid comprises the targeting peptide of SEQ ID NO: 10.

The rAAV particles comprising modified AAV9 capsid proteins, as disclosed herein, comprise three structural capsid proteins, VP1, VP2 and VP3. The three capsid proteins are alternative splice variants. The full length VP1 protein has a sequence of SEQ ID NO: 9. The VP2 protein include amino acids 138 to 736 of SEQ ID NO: 9. The VP3 protein include amino acids 203 to 736 of SEQ ID NO: 9. In some embodiments, the targeting peptide (SEQ ID NO: 10) is inserted into the AAV9 VP3 capsid protein in the rAAV particle. In some embodiments, the targeting peptide (SEQ ID NO: 10) is inserted into the AAV9 VP2 capsid protein in the rAAV particle. In some embodiments, the targeting peptide (SEQ ID NO: 10) is inserted into the AAV9 VP1 capsid protein in the rAAV particle. In some embodiments, the targeting peptide (SEQ ID NO: 10) is inserted into the VP1, VP2 and VP3 capsid proteins within the rAAV particle. In some embodiments, the targeting peptide in the modified AAV9 capsid protein is flanked by linker sequences on the N-terminal and the C-terminal end of the targeting peptide. In some embodiments, the linker sequence on the N-terminal side has the sequence AAA. In some embodiments, the linker sequence on the C-terminal side is AS.

It has been discovered that rAAV particles comprising modified AAV9 capsid proteins comprising a targeting peptide of SEQ ID NO: 10 show very high levels of transgene expression following administration to the brain (e.g., intra-CSF administration) of a subject. For instance, rAAV particles comprising modified VP1 capsid protein having a SEQ ID NO: 12 (and/or a modified VP2 capsid protein having a SEQ ID NO: 13 and/or a modified VP2 capsid protein having a SEQ ID NO: 14) show higher levels of transgene expression in the brain and spinal cord than rAAV particles comprising other capsids capable of transducing the cells of the CNS, for instance AAV9 and AAV.rh10, following intra-CSF administration of the rAAV particles. Conversely, the levels of transgene expressed in other organs such as the heart and liver are substantially lower following intra-CSF administration of rAAV particles comprising a modified AAV9 capsid comprising a targeting peptide of SEQ ID NO: 10. Moreover, high levels of expression can be achieved with lower doses of rAAV particles comprising a modified AAV9 capsid comprising a targeting peptide of SEQ ID NO: 10 relative to rAAV particles comprising other capsids capable of transducing the cells of the CNS, for instance AAV9 and AAV.rh10. Accordingly, rAAV particles comprising modified AAV9 capsid proteins comprising a targeting peptide of SEQ ID NO: 10 have an improved therapeutic index relative to rAAV particles comprising an AAV9 capsid or AAV.rh10 capsid.

Accordingly, in one aspect, the invention provides methods for treating MLD in a patient in need thereof, comprising administering to the cerebrospinal fluid (CSF) of a patient a recombinant adeno-associated virus (rAAV) viral particle comprising (1) a vector encoding an ARSA polypeptide and (2) a modified AAV9 capsid protein, wherein the modified rAAV capsid protein comprises a targeting peptide of SEQ ID NO: 10. In some embodiments, the modified AAV9 capsid protein comprising SEQ ID NO:10 is at least 90% (e.g., at least 92%, at least 95% at least 98%, at least 98,5%, at least 99%, at least 99.2%, at least 99.5%, or at least 99.8%) identical to a capsid protein having a SEQ ID NO: 12. In some embodiments, the capsid protein has SEQ ID NO: 12.

In some embodiments of the above aspects, the rAAV particle comprises a vector comprising an expression cassette flanked by one or more AAV inverted terminal repeat (ITR) sequences. In some embodiments, the expression cassette is flanked by two AAV ITRs. In some embodiments, the AAV ITRs are AAV1, AAV2, AAV3, AAV4, AAV5, AAV6, AAV7, AAV8, AAVrh8, AAVrh8R, AAV9, AAV10, AAV.rh10, AAV11, AAV12, AAV2R471A, AAV DJ, a goat AAV, bovine AAV, or mouse AAV serotype ITRs. In some embodiments, the AAV ITRs are AAV2 ITRs. In some embodiments, the vector is a self-complimenting vector. In some embodiments, the vector comprises first nucleic acid sequence encoding the ARSA polypeptide and a second nucleic acid sequence encoding a complement of the ARSA polypeptide, wherein the first nucleic acid sequence can form intrastrand base pairs with the second nucleic acid sequence along most or all of its length. In some embodiments, the first nucleic acid sequence and the second nucleic acid sequence are linked by a mutated AAV ITR, wherein the mutated AAV ITR comprises a deletion of the D region and comprises a mutation of the terminal resolution sequence.

In some aspects, the invention provides a composition comprising any of the rAAV particles described herein. In some embodiments, the composition further comprises a pharmaceutically acceptable carrier.

In some aspects, the invention provides a cell comprising any of the rAAV particles described herein. In some aspects, the invention provides a method of producing an ARSA polypeptide, the method comprising culturing a cell as described herein under conditions to produce the ARSA polypeptide. In some embodiments, the methods further comprise the step of purifying the ARSA polypeptide.

In some aspects, the invention provides methods for treating MLD in an individual in need thereof, comprising administering to the individual a rAAV particle as described herein. In some aspects, the invention provides methods for treating MLD in an individual in need thereof, comprising administering to the individual a composition as described herein. In some embodiments, the invention provides methods for treating MLD in an individual in need thereof, comprising administering to the individual the cell as described herein. In some embodiments, the individual lacks ARSA activity.

In some embodiments, the invention provides methods of increasing ARSA activity by at least about 5% in an individual in need thereof, comprising administering the individual a rAAV particle as described herein. In other embodiments, the invention provides methods of increasing ARSA activity by at least about 10% in an individual in need thereof, comprising administering the individual a rAAV particle as described herein. In other embodiments, the invention provides methods of increasing ARSA activity by at least about 20% in an individual in need thereof, comprising administering the individual a rAAV particle as described herein. In other embodiments, the invention provides methods of increasing ARSA activity by at least about 30% in an individual in need thereof, comprising administering the individual a rAAV particle as described herein. In other embodiments, the invention provides methods of increasing ARSA activity by at least about 50% in an individual in need thereof, comprising administering the individual a rAAV particle as described herein.

Administration of the rAAV particles may be conducted through various routes. In some embodiments, the administration includes direct spinal cord injection and/or intracerebral administration. In some embodiments, the administration is at a site selected from the cerebrum, medulla, pons, cerebellum, intracranial cavity, meninges surrounding the brain, dura mater, arachnoid mater, pia mater, cerebrospinal fluid (CSF) of the subarachnoid space surrounding the brain, deep cerebellar nuclei of the cerebellum, ventricular system of the cerebrum, subarachnoid space, striatum, cortex, septum, thalamus, hypothalamus, and the parenchyma of the brain. In some embodiments, the administration comprises intracerebroventricular injection into at least one cerebral lateral ventricle. In some embodiments, the administration comprises intrathecal injection in the cervical, thoracic, and/or lumbar region. In some embodiments, the administration comprises intrastriatal injection. In some embodiments, the administration comprises intrathalamic injection.

In some embodiments of the above aspects, the rAAV particle is administered via direct injection into the spinal cord, via intrathecal injection, and/or via intracisternal injection. In some embodiments, the rAAV particle is administered to more than one location of the spinal cord or cisterna magna. In some embodiments, the rAAV particle is administered to more than one location of the spinal cord. In some embodiments, the rAAV particle is administered to one or more of a lumbar subarachnoid space, thoracic subarachnoid space and a cervical subarachnoid space of the spinal cord. In some embodiments, the rAAV particle is administered to the cisterna magna.

In some embodiments of the above aspects, the rAAV particle is administered only one time to a patient in need thereof. In some embodiments, the rAAV particle is administered multiple times to a patient in need thereof (e.g., over one or more months or years). In other embodiments, the rAAV particle is administered once every year to a patient in need thereof. In other embodiments, the rAAV particle is administered twice yearly to a patient in need thereof.

In some embodiments, the invention provides kits comprising any of the rAAV particles, the compositions, or the cell as described herein. In some embodiments, the kit further comprises instructions for use; buffers and/or pharmaceutically acceptable excipients; and/or bottles, vials and/or syringes.

### BRIEF DESCRIPTION OF THE DRAWINGS

FIGS. 1A-C show increased ARSA expression in vivo with viral particles comprising two different expression cassettes, driving better clearance of toxic sulfatide deposits (C24-ST) in a mouse model of MLD (ARSA^{-/-}). FIG. 1A shows the expression cassettes used in the viral particles, one with a WPRE element and one without a WPRE element. FIG. 1B shows expression levels of ARSA following bilateral retro-orbital injection of the viral particles to mice. FIG. 1C shows C24-ST levels following bilateral retro-orbital injection of the viral particles to mice.

FIG. 2 shows that AAV.rh10-CBA-ARSA-WPRE treated late-stage MLD (ARSA^{-/-}) mice show increased ARSA-mediated sulfatase activity in brain, spinal cord and liver.

FIG. 3 depicts that AAV.rh10-CBA-ARSA-WPRE treated late-stage MLD (ARSA^{-/-}) mice show significantly reduced sulfatide levels in brain and spinal cord, 3-months post dosing.

FIG. 4 shows improved Corpus Callosum myelination measured in brain of AAV.rh10-CBA-ARSA-WPRE treated late-stage MLD (ARSA^{-/-}) mice.

FIG. 5 shows increased myelin-forming and mature oligodendrocyte cell numbers observed in brain of AAV.rh10-CBA-ARSA-WPRE treated late-stage MLD (ARSA^{-/-}) mice.

FIGS. 6A-B show the extent of ARSA protein cross-correction in brain of AAV.rh10-CBA-ARSA-WPRE treated late-stage MLD (ARSA^{-/-}) mice. FIG. 6A shows the signal overlay of matched-sagittal brain hemi-sections after three months following administration of AAV.rh10-CBA-ARSA-WPRE. FIG. 6B shows a heat map of the cross-correction factor.

FIG. 7A-B shows that AAV.rh10-CBA-ARSA-WPRE treated early-stage MLD

(ARSA^{-/-}) mice show broad CNS human ARSA (hARSA) expression and increased ARSA-mediated sulfatase activity in brain, spinal cord, dorsal root ganglions (DRGs) and liver. FIG. 7A shows that four months post dosing, broad ARSA expression was observed in sagittal brain sections by IHC against human ARSA. FIG. 7B shows that the brain, spinal cord, DRGs, and liver manifest a significant increase in ARSA activity in the animals treated with AAV.rh10-CBA-ARSA-WPRE.

FIGS. 8A-D show sulfatide levels in brain (FIG. 8A), liver (FIG. 8B), plasma (FIG. 8C) and CSF (FIG. 8D) following AAV.rh10-CBA-ARSA-WPRE dosed to early-stage (6 month) ARSA^{-/-} mice. Sulfatide levels were increased significantly in the brain, spinal cord, liver, plasma, and CSF following administration of AAV.rh10-CBA-ARSA-WPRE.

FIGS. 9A-C show that treatment with AAV.rh10-ARSA-FLAG-WPRE administration results in significantly increased sulfatase activity in the brain and spinal cord of non-human primates (NHPs) at low and high doses of the viral particles. FIG. 9A shows sulfatase activity in the brain. FIG. 9B shows sulfatase activity in the spinal cord. FIG. 9C shows representative brain sections following administration of the viral particles.

FIGS. 10A and 10B show that capsid AAV1999 displays higher transgene expression in the brain and spinal cord of NHPs, compared to AAVrh.10. FIG. 10A shows expression levels of green fluorescent protein (GFP) following administration of the rAAV particles. FIG. 10B show that across all samples, GFP expression in brain of AAV1999-treated NHPs was 93%-123% higher, as compared to AAVrh.10-treated NHPs.

FIG. 11 shows a heatmap of GFP expression in the brain following administration of AAV1999 or AAVrh.10 to NHPs.

FIG. 12 shows representative matched brain sections stained with antibodies at eGFP show robust expression in AAV1999 treated NHPs, demonstrating superior biodistribution compared to AAVrh10 (FIG. 12).

FIG. 13 shows GFP expression in the spinal cord and DRGs of NHPs following administration of particles comprising AAV1999-GFP.

FIG. 14 shows AAV1999-GFP expression in the heart and liver were lower compared to AAVrh10-GFP in treated NHPs.

FIGS. 15A-B shows AAVrh10 and AAV1999 vector genome load in various tissue following administration of AAVrh10-GFP particles or AAV1999-GFP particles. FIG. 15A shows vector genome loads in the brain, spinal cord and DRG. The vector genome load in the brain, spinal cord and DRG are lower for AAV1999 than for AAVrh10. FIG. 15B shows vector genome loads in the liver, heart, lung, kidney and spleen following administration of AAVrh10-GFP or AAV1999-GFP.

FIG. 16 shows a left-shift in the correlation between GFP expression (y-axis) and tissue vector genomes (x-axis), where in the correlation for AAV1999 (red) is left shifted indicating higher protein expression and lower (˜10 fold) tissue dose of vector compared to AAVrh.10.

FIG. 17 shows a vector map of a particular plasmid for rAAV particle production.

FIG. 18 shows that Arsa KO mice treated with AAV1999-ARSA show persistent hARSA mRNA expression. Thirteen-months post-dose, Arsa KO mice were euthanized and brain and spinal cord samples collected. RTdPCR was performed to quantify hARSA mRNA expression, normalized to mouse Hprt gene. Each data point represents a single animal. FB=formulation buffer. Error bars represent mean with standard error; Statistics: one-way ANOVA with Tukey's multiple comparison test. *<0.05; **<0.01; ***<0.001; ****<0.0001. WT+FB: wild type mice dosed with formulation buffer; KO+FB: Arsa KO mice dosed with formulation buffer; KO+AAV: Arsa KO mice dosed with AAV1999-ARSA.

FIG. 19 shows that 1AAV1999-ARSA treated Arsa KO mice show persistent ARSA-mediated sulfatase activity over time. Thirteen-months post-dose, Arsa KO mice were euthanized and brain, spinal cord, DRG, sciatic nerve, liver, and plasma samples collected. ARSA-mediated sulfatase activity was measured using the Sulfatase Activity Assay; data normalized to total protein measured by BCA assay. Each data point represents a single animal. Error bars represent mean with standard error; Statistics: One-way ANOVA with Tukey's multiple comparison test. *<0.05; **<0.01; ***<0.001; ****<0.0001.

FIG. 20 shows that 2 AAV1999-ARSA treated Arsa KO mice show significant improvement in lyso-sulfatide levels in the brain and spinal cord. Thirteen-months post-dose, Arsa KO mice were euthanized and brain, spinal cord, DRG, sciatic nerve, liver, plasma and CSF samples collected. Sulfatide levels were measured using LC-MS. Data normalized to tissue weight: converted from ng/mL (50 μL) to μg/g. Data was converted from (30 ul) ng/ml to ng/ml (plasma), and ng/ml (5 ul) to ng/ul (CSF). Each data point represents a single animal. Error bars represent mean with standard error; Statistics: one-way ANOVA with Tukey's multiple comparison test. *<0.05; **<0.01; ***<0.001; ****<0.0001.

FIG. 21 shows that Arsa KO mice treated with AAV1999-ARSA show significant improvements in expression of key inflammatory markers. Thirteen-months post-dose, Arsa KO mice were euthanized and brain and spinal cord samples collected. RTdPCR was performed to quantify Gfap, Aif1 (gene for Iba1) and Lamp1 levels, normalized to mouse Hprt gene. Each data point represents a single animal. Error bars represent mean with standard error; Statistics: one-way ANOVA with Tukey's multiple comparison test. *<0.05; **<0.01; ***<0.001; ****<0.0001.

FIG. 22 shows that treatment of Arsa KO mice with AAV1999-hARSA significantly reduced plasma Nf-L levels long-term (13-months post-dose). Thirteen-months post-dose, Arsa KO mice were euthanized, and plasma samples collected. Nf-L levels were measured by Quanterix platform. Each data point represents a single animal. Error bars represent mean with standard error; Statistics: one-way ANOVA with Tukey's multiple comparison test. *<0.05; **<0.01; ***<0.001; ****<0.0001.

FIG. 23 shows that Arsa KO mice show progressive deterioration in hearing with time; treatment with AAV1999-ARSA prevents progression of auditory phenotype: At 4-, 7-, 10- and 13-months post dose, ABR measurements were recorded via electrodes placed on the scalp of an anesthetized animal. Error bars represent mean with standard error; Statistics: Two-way ANOVA with Tukey's multiple comparison test. Asterisks denote KO+FB vs KO+AAV group. *=KO+FB vs KO+AAV; *<0.05; **<0.01; ***<0.001; ****<0.0001.

FIGS. 24A-C show that AAV1999-ARSA treated Arsa KO mice show significant reversal of MLD-associated pathology in brain and spinal cord: Thirteen-months post-dose, Arsa KO mice were euthanized, and brain (FIG. 24A), spinal cord (FIG. 24B) and DRG (FIG. 24C) samples collected for histopathology assessment. Each data point represents a single animal. Error bars represent mean with standard error; Statistics: One-way ANOVA with Tukey's multiple comparison test. *<0.05; **<0.01; ***<0.001; ****<0.0001

FIGS. 25A-C show that AAV1999-ARSA treated Arsa KO mice show persistent ARSA-mediated sulfatase activity over time. One-, two-, three- and six-months post-dosing, Arsa KO mice were euthanized and brain samples collected (FIG. 25A). Digital PCR (dPCR) was performed to quantify AAV1999-ARSA vector levels in each tissue and normalized to the Rab1a gene. (FIG. 25B and FIG. 25C) ARSA-mediated sulfatase activity was measured using the Sulfatase Activity Assay Kit (ab204731); data normalized to total protein measured by BCA assay. Each data point represents a single animal. Error bars represent mean with standard error; Statistics: Two-way ANOVA with Tukey's multiple comparison test.

FIGS. 26A-F shows that AAV1999-ARSA treated Arsa KO mice show progressive clearance of sulfatide isoforms over time. One-, two-, three- and six-months post-dosing, Arsa KO mice were euthanized, and brain samples collected. (FIG. 26A) Lyso-sulfatide, (FIG. 26B) C16-sulfatide isoform and (FIG. 26C) C18-sulfatide isoform were measured using LC-MS. Data normalized to tissue weight: converted from ng/mL (50 μL) to μg/g. (FIGS. 26D-F) to elucidate progressive nature of sulfatide clearance, data is presented as a percentage of ST remaining in KO+AAV animals, relative to the average ST remaining in KO+FB animals for (FIG. 26D) Lyso-sulfatide, (FIG. 26E) C16-sulfatide isoform and (FIG. 26F) C18-sulfatide isoform. Each data point represents a single animal. Error bars represent mean with standard error; Statistics: Two-way ANOVA with Tukey's multiple comparison test. *<0.05; **<0.01; ***<0.001; ****<0.0001.

FIG. 27 shows that total sulfatide levels in CSF and plasma of Arsa KO mice show significant reduction upon treatment with AAV1999-ARSA. One-, two-, three- and six-months post-dosing, Arsa KO mice were euthanized, and CSF and plasma samples collected. Sulfatide levels were measured using LC-MS. Data was converted from (30 ul) ng/ml to ng/ml (plasma), and ng/ml (5 ul) to ng/ul (CSF). Each data point represents a single animal. Error bars represent mean with standard error; Statistics: two-way ANOVA with Tukey's multiple comparison test.

FIG. 28 shows initial increase in plasma Nf-L levels decreased by 6 months post-dosing with AAV1999-ARSA, compared to KO+FB group. One-, two-, three- and six-months post-dosing, Arsa KO mice were euthanized, and plasma samples collected. Nf-L levels were measured by Quanterix platform. Each data point represents a single animal. Error bars represent mean with standard error; Statistics: one-way ANOVA with Tukey's multiple comparison test. *<0.05; **<0.01; ***<0.001; ****<0.0001.

FIGS. 29A-D show that microscopic findings of minimal severity found in brain AAV1999-ARSA treated Arsa KO mice. One-, two-, three- and six-months post-dosing, Arsa KO mice were euthanized, and brain samples collected for histopathology analysis. Each data point represents a single animal; findings classified under incidence and severity of (FIG. 29A) brain stem and cerebellar degeneration, (FIG. 29B) vacuolation, (FIG. 29C) infiltration and (FIG. 29D) hippocampal degeneration. Error bars represent mean with standard error; Statistics: two-way ANOVA with Tukey's multiple comparison test.

FIGS. 30A-E show that AAV1999-ARSA treated Arsa KO mice show minimal to mild findings in DRG and peripheral nerves. One-, two-, three- and six-months post-dosing, Arsa KO mice were euthanized, and DRGs (FIGS. 30A-C), spinal cord (FIGS. 30D) and sciatic nerve (FIG. 30E) were collected for histopathology analysis. Each data point represents a single animal. Error bars represent mean with standard error; Statistics: two-way ANOVA with Tukey's multiple comparison test.

FIG. 31 shows widespread dose-dependent AAV1999-ARSA vector biodistribution in NHP brain. Five-weeks post-dose of AAV1999-ARSA, NHPs were euthanized, and samples collected. The brain was hemisected with one hemisphere flash frozen; 64 tissue punches were collected from each hemisphere representing grey (47 punches) and white (17 punches) matter. Digital PCR (dPCR) was performed to quantify AAV1999 vector concentration in each tissue punch and normalized to the TUBB1 gene intron. Each data point represents VG/cell exposure for that tissue punch, either (left) averaged across all NHPs in that group or (right) presented by individual brain regions. Error bars represent mean with standard error; Statistics: one-way ANOVA with Tukey's multiple comparison test. Doses per NHP.

FIG. 32 shows widespread dose-dependent hARSA mRNA expression across 19 grey matter brain regions in NHP. Five-weeks post-dose, NHPs were euthanized, and samples collected. The brain was hemisected with one hemisphere flash frozen; 47 tissue punches were collected from each hemisphere representing 19 grey matter regions. Reverse transcription digital PCR (RTdPCR) was performed to quantify hARSA mRNA expression, normalized to Macaca fascicularis Hypoxanthine Phosphoribosyltransferase 1 (HPRT) gene. Each data point represents normalized hARSA expression in that tissue punch, either (left) averaged across all NHPs in that group or (right) presented by individual brain regions. Left: scatterplot representing median; Right: error bars represent mean with standard error; Statistics: one-way ANOVA with Tukey's multiple comparison test.

FIGS. 33A-C shows widespread dose-dependent hARSA protein expression across 19 grey matter brain regions in NHP. Five-weeks post-dose AAV1999-ARSA, NHPs were euthanized, and samples collected. The brain was hemisected with one hemisphere flash frozen; 30 tissue punches were collected from each hemisphere representing grey matter. LC-MS was performed to quantify human ARSA protein levels. Each data point represents the amount of human ARSA in that tissue punch, average across three animals in that group, either presented as an (FIG. 33A) averaged across all NHPs in that group, (FIG. 33B) presented per individual brain regions, or (FIG. 33C) as a correlation to vector genome in those punches. Error bars represent mean with standard error. Data from 1e10 VG/gm (7.5e11 VG) and 3.3e10 VG/gm (2.5e12 VG) were below limits of detection and have been excluded. Left: scatterplot representing median; Right: error bars represent mean with standard error; Statistics: one-way ANOVA with Tukey's multiple comparison test.

FIGS. 34A-B show uniform dose-dependent AAV1999 vector biodistribution and hARSA mRNA expression along the spinal rostral-caudal axis. Five-weeks post-dose of AAV1999-ARSA, NHPs were euthanized, and samples collected. Eight spinal cord segments with adjacent DRGs, two each from cervical, upper thoracic, lower thoracic and lumbar were flash frozen and DNA/RNA isolated. (FIG. 34A) Digital PCR (dPCR) was performed to quantify AAV1999 vector concentration and normalized to the TUBB1 gene intron. (FIG. 34B) RTdPCR was performed to quantify hARSA mRNA expression, normalized to endogenous HPRT gene. Each data point represents background subtracted VG/cell exposure or normalized hARSA expression for that sample, averaged across all NHPs in that group. Error bars represent mean with standard error.

FIG. 35 shows AAV1999-ARSA vector biodistribution to visceral organs and peripheral nerve. Five-weeks post-dose, NHPs were euthanized, and samples from visceral organs collected. Digital PCR (dPCR) was performed to quantify AAV1999 vector concentration in each tissue punch and normalized to the TUBB1 gene intron. Each data point represents VG/cell exposure for that tissue punch, averaged across all NHPs in that group. Error bars represent mean with standard error.

FIG. 36 shows human ARSA protein expression in NHP brain likely to meet therapeutic threshold @1e11 and 3.3e11 VG/gm brain weight dose. Five-weeks post-dose of AAV1999-ARSA, NHPs were euthanized, and samples collected. The brain was hemisected with one hemisphere flash frozen; 30 tissue punches were collected from each hemisphere representing grey matter. LC-MS was performed to quantify human and endogenous Macaca fascicularis cyARSA protein levels, and presented as a percentage (huARSA/cyARSA). Each data point is represented as a ratio of hARSA to cynomolgus cyARSA protein in that tissue punch, average across three animals in that group, either presented as an (left) averaged across all NHPs in that group or (right) presented per individual brain regions. Error bars represent mean with standard error.

FIGS. 37A-B shows that AAV1999-ARSA effective doses are therapeutically meaningful. FIG. 37A shows LC-MS was performed to quantify human ARSA protein levels in 12 brain regions from 7 health 3-8 yr old organ-donors. Tissues were procured from the NIH brain bank. FIG. 37B shows five-weeks post-dose, NHPs were euthanized, and samples collected. The brain was hemisected with one hemisphere flash frozen; 30 tissue punches were collected from each hemisphere representing grey matter. LC-MS was performed to quantify human ARSA protein levels. Mean ARSA protein in each group is presented. The Sponsor presents the percentage of human ARSA in NHPs, relatable to human ARSA in brain of health organ-donors. Error bars represent mean with standard error.

FIG. 38 shows that no clinical signs (functional or behavioral deficits) observed in NHPs at week 1 or week 5 (necropsy) post-dosing of AAV1999-ARSA in either dosing groups. NHPs were tested for 35 functional and behavioral parameters pre-dose, day 7 post-dose and prior to necropsy (week 5 post-dose). Findings were scored as either ‘in normal range’ or ‘abnormal clinical findings’. Graphed below are findings at necropsy. Functional and behavioral assessment performed by performed by CRL veterinarian.

FIGS. 39A-C show that intra-CM injection of AAV1999-ARSA was well tolerated, resulted in expected Nf-L elevation; AAV1999-ARSA did not trigger innate nor cell-mediated immune responses. As shown in FIG. 39A, pre-dose and at necropsy, CSF was collected and analyzed for Nf-L levels by Quanterix platform. Each data point represented Nf-L levels in that sample, averaged across all NHPs in that group. As shown in FIG. 39B, Plasma was isolated pre-dose and at days 2, -4, 7, 14 post dose and at necropsy. The Luminex assay was used to determine the concentration of IL-1b, IL-1RA, IL-6, IL-10, IL-12/23 (p40), IL-15, IL-18, IFN-g, TNF-a, G-CSF, MCP-1, MIP-1b, GM-CSF, IL-2, IL-4, IL-5, IL-8, IL-13 and IL-17A. Each data point represented cytokine concentration in that sample, averaged across all NHPs in that group. As shown in FIG. 39C, PBMCs isolated from animals in the 1e11 and 3.3e11 VG/gm brain weight dosing groups were subjected to IFN-γ ELISpot. Error bars represent mean with standard error; Statistics: one-way ANOVA with *Tukey's and ^{#}Dunnett's multiple comparison test.

FIG. 40 shows that AAV1999-ARSA treated NHPs show up to mild microscopic findings in brain across four doses. Five-weeks post-dose, NHPs were euthanized, and samples collected. The brain was hemisected with one hemisphere fixed for histopathological assessment. Each data point represents the incidence and severity of microscopic findings, averaged across all animals in that dosing group, measured in the categories of mononuclear infiltration, neuronal or nerve fiber degeneration, and gliosis. Error bars represent mean with standard error.

FIGS. 41A-41B shows AAV1999-ARSA treated NHPs show up to moderate microscopic findings in spinal cord, radial and femoral nerves of NHPs; marked findings in sciatic nerve. Five-weeks post-dose, NHPs were euthanized, and samples collected for histopathological assessment. Each data point represents the incidence and severity of microscopic findings, averaged across all animals in that dosing group, measured in the categories of mononuclear infiltration, neuronal or nerve fiber degeneration, and gliosis. Error bars represent mean with standard error.

FIGS. 42A-42B show AAV1999-ARSA treated NHPs show up to mild findings in DRGs, when averaged across 8 segments along the spinal rostral-caudal axis. Five-weeks post-dose, NHPs were euthanized, and samples collected for histopathological assessment. In the left panels, each data point represents the average incidence and severity of microscopic findings averaged across DRGs from 8 spinal segments, across all animals in that dosing group. In the right panels, each data point represents the incidence and severity of microscopic findings per DRG, averaged across all animals in that dosing group. Categories measured are (FIG. 42A) degeneration and necrosis and (FIG. 42B) mononuclear infiltration. Error bars represent mean with standard error.

### DETAILED DESCRIPTION

In some aspects, the invention provides expression cassettes, recombinant adeno-associated virus (rAAV) vectors, and viral particles and pharmaceutical compositions comprising the transgene encoding an ARSA polypeptide. In further aspects, the invention provides methods for treating metachromatic leukodystrophy (MLD), for example, by increasing ARSA activity, reducing sulfatide accumulation in the brain, spinal cord, liver, plasma and CSF, increasing and normalizing myelination in the corpus callosum, increasing oligodendrocyte populations in the brain, and improving CNS pathology, consequent to sulfatide clearance in brain regions. In yet further aspects, the invention provides kits for treating MLD in an individual with an expression cassette of the present disclosure.

### Definitions

A “vector,” as used herein, refers to a recombinant plasmid or virus that comprises a nucleic acid to be delivered into a host cell, either in vitro or in vivo.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one and in embodiments two, inverted terminal repeat sequences (ITRs).

A “recombinant AAV vector (rAAV vector)” refers to a polynucleotide vector comprising one or more heterologous sequences (i.e., nucleic acid sequence not of AAV origin) that are flanked by at least one, and in embodiments two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

“Chicken β-actin (CBA) promoter” refers to a polynucleotide sequence derived from a chicken β-actin gene (e.g., Gallus beta actin, represented by GenBank Entrez Gene ID 396526). As used herein, “chicken β-actin promoter” may refer to a promoter containing a cytomegalovirus (CMV) early enhancer element, the promoter and first exon and intron of the chicken β-actin gene, and the splice acceptor of the rabbit beta-globin gene, such as the sequences described in Miyazaki, J. et al. (1989) Gene 79(2):269-77. As used herein, the term “CAG promoter” may be used interchangeably. As used herein, the term “CMV early enhancer/chicken beta actin (CAG) promoter” may be used interchangeably.

The terms “genome particles (gp),” “genome equivalents,” or “genome copies” as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Virol., 62:1963-1973.

The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins.

“AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

A “helper virus” for AAV refers to a virus that allows AAV (which is a defective parvovirus) to be replicated and packaged by a host cell. A helper virus provides “helper functions” which allow for the replication of AAV. A number of such helper viruses have been identified, including adenoviruses, herpesviruses and, poxviruses such as vaccinia and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions. Baculoviruses available from depositories include Autographa californica nuclear polyhedrosis virus.

A preparation of rAAV is said to be “substantially free” of helper virus if the ratio of infectious AAV particles to infectious helper virus particles is at least about 10²:1; at least about 10⁴:1, at least about 10⁶:1; or at least about 10⁸:1 or more. In some embodiments, preparations are also free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

An “effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” is an approach for obtaining beneficial or desired clinical results. For purposes of this disclosure, beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the disclosure described herein include “comprising,” “consisting,” and/or “consisting essentially of” aspects and embodiments.

### Expression Cassettes

In some embodiments, the transgene encoding an ARSA polypeptide is codon-optimized. In some embodiments, the transgene encoding an ARSA polypeptide is codon optimized for expression in a particular cell, such as a eukaryotic cell. Eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon usage tables are readily available, for example, at the “Codon Usage Database”, and these tables can be adapted in a number of ways (see, e.g., Nakamura, Y. et al. (2000) Nucleic Acids Res. 28:292). Computer algorithms for codon optimizing a particular sequence for expression in a particular host cell are also available, such as Gene Forge (Aptagen; Jacobus, Pa.), DNA2.0, GeneArt (GA) or Genscript (GS) and a GS algorithm combined with reduction in CpG content. In some embodiments, a transgene encoding the ARSA polypeptide is codon optimized using the GA algorithm. In some embodiments, the transgene encoding the ARSA polypeptide has a sequence of SEQ ID NO: 1.

In some embodiments, the expression cassette further comprises an intron. A variety of introns for use in the invention are known to those of skill in the art, and include the MVM intron, the F IX truncated intron 1, the β-globin SD/immunoglobin heavy chain SA, the adenovirus SD/immunoglobin SA, the SV40 late SD/SA (19S/16S), and the hybrid adenovirus SD/IgG SA. (Wu et al. 2008, Kurachi et al., 1995, Choi et al. 2014, Wong et al., 1985, Yew et al. 1997, Huang and Gorman (1990). In some embodiments, the intron is a chicken β-actin (CBA)/rabbit β-globin hybrid intron. In some embodiments, intron is a chicken β-actin (CBA)/rabbit β-globin hybrid promoter and intron where all the ATG sites are removed to minimize false translation start sites. In some embodiments the intron is an MVM intron, a F IX truncated intron 1, a β-globin SD/immunoglobin heavy chain SA, an adenovirus SD/immunoglobin SA, a SV40 late SD/SA (19S/16S), or a hybrid adenovirus SD/IgG SA. In some embodiments, the intron is a chicken β-actin (CBA)/rabbit β-globin hybrid intron.

In some embodiments, the expression cassette further comprises a polyadenylation signal. In some embodiments, the polyadenylation signal is a bovine growth hormone polyadenylation signal, an SV40 polyadenylation signal, or a HSV TK pA. In some embodiments, the polyadenylation signal is a synthetic polyadenylation signal as described in Levitt, N et al. (1989), Genes Develop. 3:1019-1025.

In some embodiments, the expression cassette comprises a stuffer nucleic acid. In some embodiments, the stuffer nucleic acid may comprise a sequence that encodes a reporter polypeptide. As will be appreciated by those of skill in the art, the stuffer nucleic acid may be located in a variety of regions within the nucleic, and may be comprised of a continuous sequence (e.g., a single stuffer nucleic acid in a single location) or multiple sequences (e.g., more than one stuffer nucleic acid in more than one location (e.g., 2 locations, 3 locations, etc.) within the nucleic acid. In some embodiments, the stuffer nucleic acid may be located downstream of the transgene encoding the ARSA polypeptide. In embodiments, the stuffer nucleic acid may be located upstream of the transgene encoding the ARSA polypeptide (e.g., between the promoter and the transgene). As will also be appreciated by those of skill in the art a variety of nucleic acids may be used as a stuffer nucleic acid. In some embodiments, the stuffer nucleic acid comprises all or a portion of a human alpha-1-antitrypsin (AAT) stuffer sequence or a C16 P1 chromosome 16 P1 clone (human C16) stuffer sequence. In some embodiments, the stuffer sequence comprises all or a portion of a gene. For example, the stuffer sequence comprises a portion of the human AAT sequence. One skilled in the art would recognize that different portions of a gene (e.g., the human AAT sequence) can be used as a stuffer fragment. For example, the stuffer fragment may be from the 5′ end of the gene, the 3′ end of the gene, the middle of a gene, a non-coding portion of the gene (e.g., an intron), a coding region of the gene (e.g. an exon), or a mixture of non-coding and coding portions of a gene. One skilled in the art would also recognize that all or a portion of stuffer sequence may be used as a stuffer sequence. In some embodiments, the stuffer sequence is modified to remove internal ATG codons.

In some embodiments, the expression cassette is incorporated into a vector. In some embodiments, the expression cassette is incorporated into a viral vector. In some embodiments, the viral vector is a rAAV vector as described herein.

### Vectors and Viral Particles

In certain aspects, the expression cassette for expressing an ARSA polypeptide (e.g., a wild type human ARSA polypeptide) is contained in a vector. In some embodiments, the present invention contemplates the use of a recombinant viral genome for introduction of nucleic acid sequences encoding the ARSA polypeptide for packaging into a viral particle, e.g., a viral particle described below. The recombinant viral genome may include any element to establish the expression of the ARSA polypeptide, for example, a promoter, an ITR, a ribosome binding element, terminator, enhancer, selection marker, intron, polyA signal, and/or origin of replication. Exemplary viral genome elements and delivery methods for viral particles are described in greater detail below.

### Non-Viral Delivery Systems

Conventional non-viral gene transfer methods may also be used to introduce nucleic acids into cells or target tissues. Non-viral vector delivery systems include DNA plasmids, naked nucleic acid, and nucleic acid complexed to a delivery system. For example, the vector may be complexed to a lipid (e.g., a cationic or neutral lipid), a liposome, a polycation, a nanoparticle, or an agent that enhances the cellular uptake of nucleic acid. The vector may be complexed to an agent suitable for any of the delivery methods described herein. In some embodiments, the nucleic acid comprises one or more viral ITRs (e.g., AAV ITRs).

### Viral Particles

In some embodiments, the vector comprising the expression cassette for expressing an ARSA polypeptide (e.g., a wild type human ARSA polypeptide) is a recombinant viral vector. Some examples of recombinant viral vectors include AAV, lentivirus and adenovirus. In one aspect, the viral vector is a recombinant adeno-associated virus (rAAV) vector. In some embodiments, the expression cassette for expressing an ARSA polypeptide (e.g., a wild type human ARSA polypeptide) is flanked by one or more AAV inverted terminal repeat (ITR) sequences. In some embodiments, the viral particle is a recombinant AAV particle comprising an expression cassette for expressing an ARSA polypeptide flanked by one or two ITRs. In some embodiments, the expression cassette for expressing an ARSA polypeptide is flanked by two AAV ITRs.

In some embodiments, the expression cassette for expressing an ARSA polypeptide of the present disclosure operatively linked components in the direction of transcription, control sequences including transcription initiation and termination sequences, thereby forming an expression cassette. The expression cassette is flanked on the 5′ and 3′ end by at least one functional AAV ITR sequence. By “functional AAV ITR sequences” it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan et al., Gene Ther., 2009, 16:10-16, all of which are incorporated herein in their entirety by reference. For practicing some aspects of the invention, the recombinant vectors comprise at least all of the sequences of AAV essential for encapsidation and the physical structures for infection by the rAAV. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810.

Use of any AAV serotype is considered within the scope of the present invention. In some embodiments, a rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAV.rh10, AAV11, AAV12, a goat AAV, bovine AAV, or mouse AAV ITRs or the like. In some embodiments, the nucleic acid in the AAV comprises an ITR of AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, a goat AAV, bovine AAV, or mouse AAV or the like. In certain embodiments, the AAV ITRs are AAV2 ITRs.

In some embodiments, a vector may include a stuffer nucleic acid. In some embodiments, the stuffer nucleic acid may encode a green fluorescent protein (GFP). In some embodiments, the stuffer nucleic acid may be located 3′ to expression cassette for expressing an ARSA polypeptide of the present disclosure.

In some aspects, the invention provides viral particles comprising a recombinant self-complementing genome. In some embodiments, the vector is a self-complementary vector. AAV viral particles with self-complementing genomes and methods of use of self-complementing AAV genomes are described in U.S. Pat. Nos. 6,596,535; 7,125,717; 7,765,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands of a transgene). In some embodiments, the invention provides an AAV viral particle comprising an AAV genome, wherein the rAAV genome comprises a first heterologous polynucleotide sequence (e.g., the coding strand of the ARSA polypeptide of the invention) and a second heterologous polynucleotide sequence (e.g., the noncoding or antisense strand of the ARSA polypeptide of the present disclosure) wherein the first heterologous polynucleotide sequence can form intrastrand base pairs with the second polynucleotide sequence along most or all of its length.

In some embodiments, the first heterologous polynucleotide sequence and a second heterologous polynucleotide sequence are linked by a sequence that facilitates intrastrand basepairing; e.g., a hairpin DNA structure. Hairpin structures are known in the art, for example in siRNA molecules. In some embodiments, the first heterologous polynucleotide sequence and a second heterologous polynucleotide sequence are linked by a mutated ITR (e.g., the right ITR). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

In some embodiments, the first heterologous nucleic acid sequence and a second heterologous nucleic acid sequence are linked by a mutated ITR (e.g., the right ITR). In some embodiments, the ITR comprises the polynucleotide sequence 5′-CACTCCCTCTCTGCGCGCTCGCTCGCTCACTGAGGCCGGGCGACCAAAGGTCGCCCACGCCCGGGCTTTGCCCGGGCG-3′ (SEQ ID NO:15). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

In some embodiments, the vector is encapsidated in a viral particle. In some embodiments, the viral particle is a recombinant AAV viral particle comprising a recombinant AAV vector. Different AAV serotypes are used to optimize transduction of particular target cells or to target specific cell types within a particular target tissue (e.g., brain or spinal). A rAAV particle can comprise viral proteins and viral nucleic acids of the same serotype or a mixed serotype. For example, in some embodiments a rAAV particle can comprise modified AAV1999 capsid proteins and at least one AAV2 ITR or it can comprise modified AAV1999 capsid proteins and at least one AAV1 ITR. Any combination of AAV serotypes for production of a rAAV particle is provided herein as if each combination had been expressly stated herein.

The capsid of AAV (e.g., AAV9, AAV1999, etc.) is known to include three capsid proteins: VP1, VP2, and VP3. These proteins contain significant amounts of overlapping amino acid sequence and unique N-terminal sequences. An AAV9 capsid includes 60 subunits arranged by icosahedral symmetry. AAV9 includes VP1, VP2, and VP3 capsid proteins in a ratio of about 5:5:50. The VP proteins of AAV9 are products of the structural protein-encoding open reading frame of the genome, designated cap, VP1 (˜82 kDa) and VP2 (˜73 kDa), which are the minor capsid proteins, and VP3 (˜61 kDa), the major capsid protein. Due to the utilization of both alternative splicing and leaky scanning, when expressed, the individual VPs share a C terminus that encompasses the entire VP3, while VP1 and VP2 are N-terminal VP3 extensions. VP1 and VP2 share a region of ˜73 amino acids amino acids which is extended by an additional ˜137 amino acids in VP1, designated the VP1 unique region (VP1u). See Penzes et al., (2021), Journal of Virology 95(19)e0084321. In some embodiments of the modified AAV9 capsid proteins disclosed herein, the targeting peptide (e.g., SEQ ID NO:10) is incorporated into VP1. In some embodiments of the modified AAV9 capsid proteins disclosed herein, the targeting peptide (e.g., SEQ ID NO:10) is incorporated into VP2. In some embodiments of the modified AAV9 capsid proteins disclosed herein, the targeting peptide (e.g., SEQ ID NO:10) is incorporated into VP3. In some embodiments of the modified AAV9 capsid proteins disclosed herein, the targeting peptide (e.g., SEQ ID NO:10) is incorporated into VP1, VP2 and VP3.

In some embodiments, the disclosure provides rAAV particles for intra-CSF administration of an ARSA polypeptide that comprise a modified AAV9 capsid protein comprising a targeting peptide that targets the rAAV particles to the brain. In particular embodiments, the targeting peptide of the modified AAV9 capsids are inserted after residue 588 of the AAV9 structural protein. In some embodiments, the targeting peptide has SEQ ID NO: 10. In some embodiments, the targeting peptide is flanked by linker sequences on the N-terminal and the C-terminal end of the targeting peptide. In some embodiments, the linker sequence on the N-terminal side has the sequence AAA. In some embodiments, the linker sequence on the C-terminal side is AS. In some embodiments, the full sequence inserted after residue 588 of the AAV9 capsid structural protein has SEQ ID NO: 11.

In some embodiments, the full modified AAV9 capsid structural protein (VP1) has a sequence of SEQ ID NO: 12. In some embodiments, the full modified AAV9 capsid structural protein (VP1) is at least 90% (e.g., at least 92%, at least 95%, at least 98%, at least 98.5%, at least 99%, at least 99.2%, at least 99.5%, or at least 99.8%) identical to SEQ ID NO: 12, wherein the modified AAV9 structural capsid comprises the targeting peptide of SEQ ID NO: 10.

In some embodiments, the modified VP2 capsid of AAV9 capsid has a sequence of SEQ ID NO: 13. In some embodiments, the modified VP2 capsid of AAV9 has a sequence that is at least 90% (e.g., at least 92%, at least 95%, at least 98%, at least 98.5%, at least 99%, at least 99.2%, at least 99.5%, or at least 99.8%) identical to SEQ ID NO: 13, wherein the modified AAV9 structural capsid comprises the targeting peptide of SEQ ID NO: 10.

In some embodiments, the modified VP3 capsid of AAV9 capsid has a sequence of SEQ ID NO: 14. In some embodiments, the modified VP3 capsid of AAV9 has a sequence that is at least 90% (e.g., at least 92%, at least 95%, at least 98%, at least 98.5%, at least 99%, at least 99.2%, at least 99.5%, or at least 99.8%) identical to SEQ ID NO: 14, wherein the modified AAV9 structural capsid comprises the targeting peptide of SEQ ID NO: 10.

### Production of AAV Particles

Numerous methods are known in the art for production of rAAV vectors, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids (Urabe, M. et al., (2002) Human Gene Therapy 13(16):1935-1943; Kotin, R. (2011) Hum Mol Genet. 20(R1): R2-R6). rAAV production cultures for the production of rAAV virus particles all require; 1) suitable host cells, 2) suitable helper virus function, 3) AAV rep and cap genes and gene products; 4) a nucleic acid (such as a therapeutic nucleic acid) flanked by at least one AAV ITR sequences (e.g., an AAV genome encoding an ARSA polypeptide); and 5) suitable media and media components to support rAAV production. In some embodiments, the suitable host cell is a primate host cell. In some embodiments, the suitable host cell is a human-derived cell lines such as HeLa, A549, 293, or Perc.6 cells. In some embodiments, the suitable helper virus function is provided by wild-type or mutant adenovirus (such as temperature sensitive adenovirus), herpes virus (HSV), baculovirus, or a plasmid construct providing helper functions. In some embodiments, the AAV rep and cap gene products may be from any AAV serotype. In general, but not obligatory, the AAV rep gene product is of the same serotype as the ITRs of the rAAV vector genome as long as the rep gene products may function to replicated and package the rAAV genome. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors. In some embodiments, the AAV helper functions are provided by adenovirus or HSV. In some embodiments, the AAV helper functions are provided by baculovirus and the host cell is an insect cell (e.g., Spodoptera frugiperda (Sf9) cells).

One method for producing rAAV particles is the triple transfection method. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified. As such, in some embodiments, the rAAV particle was produced by triple transfection of a nucleic acid encoding the rAAV vector, a nucleic acid encoding AAV rep and cap, and a nucleic acid encoding AAV helper virus functions into a host cell, wherein the transfection of the nucleic acids to the host cells generates a host cell capable of producing rAAV particles.

In some embodiments, rAAV particles may be produced by a producer cell line method (see Martin et al., (2013) Human Gene Therapy Methods 24:253-269; U.S. PG Pub. No. US2004/0224411; and Liu, X. L. et al. (1999) Gene Ther. 6:293-299). Briefly, a cell line (e.g., a HeLa, 293, A549, or Perc.6 cell line) may be stably transfected with a plasmid containing a rep gene, a capsid gene, and a vector genome comprising a promoter-heterologous nucleic acid sequence (e.g., an ARSA polypeptide). Cell lines may be screened to select a lead clone for rAAV production, which may then be expanded to a production bioreactor and infected with a helper virus (e.g., an adenovirus or HSV) to initiate rAAV production. Virus may subsequently be harvested, adenovirus may be inactivated (e.g., by heat) and/or removed, and the rAAV particles may be purified. As such, in some embodiments, the rAAV particle was produced by a producer cell line comprising one or more of nucleic acid encoding the rAAV vector, a nucleic acid encoding AAV rep and cap, and a nucleic acid encoding AAV helper virus functions. As described herein, the producer cell line method may be advantageous for the production of rAAV particles with an oversized genome, as compared to the triple transfection method.

In some embodiments, the nucleic acid encoding AAV rep and cap genes and/or the rAAV genome are stably maintained in the producer cell line. In some embodiments, nucleic acid encoding AAV rep and cap genes and/or the rAAV genome is introduced on one or more plasmids into a cell line to generate a producer cell line. In some embodiments, the AAV rep, AAV cap, and rAAV genome are introduced into a cell on the same plasmid. In other embodiments, the AAV rep, AAV cap, and rAAV genome are introduced into a cell on different plasmids. In some embodiments, a cell line stably transfected with a plasmid maintains the plasmid for multiple passages of the cell line (e.g., 5, 10, 20, 30, 40, 50 or more than 50 passages of the cell). For example, the plasmid(s) may replicate as the cell replicates, or the plasmid(s) may integrate into the cell genome. A variety of sequences that enable a plasmid to replicate autonomously in a cell (e.g., a human cell) have been identified (see, e.g., Krysan, P. J. et al. (1989) Mol. Cell Biol. 9:1026-1033). In some embodiments, the plasmid(s) may contain a selectable marker (e.g., an antibiotic resistance marker) that allows for selection of cells maintaining the plasmid. Selectable markers commonly used in mammalian cells include without limitation blasticidin, G418, hygromycin B, zeocin, puromycin, and derivatives thereof. Methods for introducing nucleic acids into a cell are known in the art and include without limitation viral transduction, cationic transfection (e.g., using a cationic polymer such as DEAE-dextran or a cationic lipid such as lipofectamine), calcium phosphate transfection, microinjection, particle bombardment, electroporation, and nanoparticle transfection (for more details, see e.g., Kim, T. K. and Eberwine, J. H. (2010) Anal. Bioanal. Chem. 397:3173-3178).

In some embodiments, the nucleic acid encoding AAV rep and cap genes and/or the rAAV genome are stably integrated into the genome of the producer cell line. In some embodiments, nucleic acid encoding AAV rep and cap genes and/or the rAAV genome is introduced on one or more plasmids into a cell line to generate a producer cell line. In some embodiments, the AAV rep, AAV cap, and rAAV genome are introduced into a cell on the same plasmid. In other embodiments, the AAV rep, AAV cap, and rAAV genome are introduced into a cell on different plasmids. In some embodiments, the plasmid(s) may contain a selectable marker (e.g., an antibiotic resistance marker) that allows for selection of cells maintaining the plasmid. Methods for stable integration of nucleic acids into a variety of host cell lines are known in the art. For example, repeated selection (e.g., through use of a selectable marker) may be used to select for cells that have integrated a nucleic acid containing a selectable marker (and AAV cap and rep genes and/or a rAAV genome). In other embodiments, nucleic acids may be integrated in a site-specific manner into a cell line to generate a producer cell line. Several site-specific recombination systems are known in the art, such as FLP/FRT (see, e.g., O'Gorman, S. et al. (1991) Science 251:1351-1355), Cre/loxP (see, e.g., Sauer, B. and Henderson, N. (1988) Proc. Natl. Acad. Sci. 85:5166-5170), and phi C31-att (see, e.g., Groth, A.C. et al. (2000) Proc. Natl. Acad Sci. 97:5995-6000).

In some embodiments, the producer cell line is derived from a primate cell line (e.g., a non-human primate cell line, such as a Vero or FRhL-2 cell line). In some embodiments, the cell line is derived from a human cell line. In some embodiments, the producer cell line is derived from HeLa, 293, A549, or PERC.6® (Crucell) cells. For example, prior to introduction and/or stable maintenance/integration of nucleic acid encoding AAV rep and cap genes and/or the oversized rAAV genome into a cell line to generate a producer cell line, the cell line is a HeLa, 293, A549, or PERC.6® (Crucell) cell line, or a derivative thereof.

In some embodiments, the producer cell line is adapted for growth in suspension. As is known in the art, anchorage-dependent cells are typically not able to grow in suspension without a substrate, such as microcarrier beads. Adapting a cell line to grow in suspension may include, for example, growing the cell line in a spinner culture with a stirring paddle, using a culture medium that lacks calcium and magnesium ions to prevent clumping (and optionally an antifoaming agent), using a culture vessel coated with a siliconizing compound, and selecting cells in the culture (rather than in large clumps or on the sides of the vessel) at each passage. For further description, see, e.g., ATCC frequently asked questions document (available at www.atcc.org/Global/FAQs/9/1/Adapting%20a%20monolayer%2Ocell%201ine%20to%20suspension-40.aspx) and references cited therein.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) a rAAV pro-vector comprising a nucleic acid encoding a heterologous nucleic acid as described herein flanked by at least one AAV ITR, and (iii) an AAV helper function; and (b) recovering the rAAV particles produced by the host cell. In some embodiments, said at least one AAV ITR is selected from the group consisting of AAV1, AAV2, AAV3, AAV4, AAV5, AAV6, AAV7, AAV8, AAVrh8, AAVrh8R, AAV9, AAV10, AAVrh10, AAV11, AAV12, a goat AAV, bovine AAV, or mouse AAV serotype ITRs or the like. For example, in some embodiments, the AAV serotype is AAV1, AAV2, AAV5, AAV6, AAV7, AAV8, AAVrh8, AAVrh8R, AAV9, AAV10, or AAVrh10. In certain embodiments, the nucleic acid in the AAV comprises an AAV2 ITR. In some embodiments, said encapsidation protein is selected from the group consisting of AAV1, AAV2, AAV3, AAV4, AAV5, AAV6, AAV7, AAV8, AAVrh8, AAVrh8R, AAV9, AAV1999. AAV10, AAVrh10, AAV11, AAV12, AAV2R471A, AAV2/2-7m8, AAV DJ, AAV2 N587A, AAV2 E548A, AAV2 N708A, AAV V708K, goat AAV, AAV1/AAV2 chimeric, bovine AAV, mouse AAV capsid, rAAV2/HBoV1 serotype, AAV-XL32, or AAV-XL32.1 capsid proteins or mutants thereof. In some embodiments, the encapsidation protein is an AAV8 capsid protein. In some embodiments, the rAAV particles comprise an AAV9 capsid and a recombinant genome comprising AAV2 ITRs, and nucleic acid encoding a therapeutic transgene/nucleic acid (e.g., an expression cassette for expressing an ARSA polypeptide). In some embodiments, the rAAV particles comprise an AAV1999 capsid and a recombinant genome comprising AAV2 ITRs, and nucleic acid encoding a therapeutic transgene/nucleic acid (e.g., an expression cassette for expressing an ARSA polypeptide).

Suitable rAAV production culture media of the present invention may be supplemented with serum or serum-derived recombinant proteins at a level of 0.5%-20% (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the invention may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the rAAV production culture harvest is clarified to remove host cell debris. In some embodiments, the production culture harvest is clarified by filtration through a series of depth filters including, for example, a grade DOHC Millipore Millistak+ HC Pod Filter, a grade A1HC Millipore Millistak+ HC Pod Filter, and a 0.2 μm Filter Opticap XL1O Millipore Express SHC Hydrophilic Membrane filter. Clarification can also be achieved by a variety of other standard techniques known in the art, such as, centrifugation or filtration through any cellulose acetate filter of 0.2 μm or greater pore size known in the art.

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. In some embodiments, the method comprises all the steps in the order as described below. Methods to purify rAAV particles are found, for example, in Xiao et al., (1998) Journal of Virology 72:2224-2232; U.S. Pat. Nos. 6,989,264 and 8,137,948; and WO 2010/148143.

### Methods of Treatment

Certain aspects of the present disclosure relate to methods of treating MLD and/or increasing levels of an ARSA polypeptide in an individual in need thereof. In some embodiments, the invention provides methods of treating MLD by administering an effective amount of an expression cassette (e.g., an expression cassette delivered in a rAAV particle) for expressing an ARSA polypeptide of the present disclosure. In some embodiments, the ARSA polypeptide is a wild type ARSA polypeptide. The expression cassette (e.g., expression cassette delivered in a rAAV particle) for expressing an ARSA polypeptide may be administered through various routes. In some embodiments, the administration includes direct spinal cord injection and/or intracerebral administration. In some embodiments, the administration is at a site selected from the cerebrum, medulla, pons, cerebellum, intracranial cavity, meninges surrounding the brain, dura mater, arachnoid mater, pia mater, cerebrospinal fluid (CSF) of the subarachnoid space surrounding the brain, deep cerebellar nuclei of the cerebellum, ventricular system of the cerebrum, subarachnoid space, striatum, cortex, septum, thalamus, hypothalamus, and the parenchyma of the brain. In some embodiments, the administration comprises intracerebroventricular injection into at least one cerebral lateral ventricle. In some embodiments, the administration comprises intrathecal injection in the cervical, thoracic, and/or lumbar region. In some embodiments, the administration comprises intrastriatal injection. In some embodiments, the administration comprises intrathalamic injection.

An effective amount of rAAV (in some embodiments in the form of particles) is administered, depending on the objectives of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells of the desired tissue type, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. The rAAV composition may be administered by one or more administrations, either during the same procedure or spaced apart by days, weeks, months, or years. One or more of any of the routes of administration described herein may be used. In some embodiments, multiple vectors may be used to treat the human.

In some embodiments of the above aspects, the rAAV is administered via direct injection into the spinal cord, via intrathecal injection, or via intracisternal injection. In some embodiments, the rAAV is administered to more than one location of the spinal cord or cisterna magna. In some embodiments, the rAAV is administered to more than one location of the spinal cord. In some embodiments, the rAAV is administered to one or more of a lumbar subarachnoid space, thoracic subarachnoid space and a cervical subarachnoid space of the spinal cord. In some embodiments, the rAAV is administered to the cisterna magna.

Methods to identify cells transduced by AAV viral particles are known in the art; for example, immunohistochemistry or the use of a marker such as enhanced green fluorescent protein can be used to detect transduction of viral particles; for example viral particles comprising a rAAV capsid with one or more substitutions of amino acids.

In some embodiments, an effective amount of rAAV particles is administered to more than one location simultaneously or sequentially. In other embodiments, an effective amount of rAAV particles is administered to a single location more than once (e.g., repeated). In some embodiments, multiple injections of rAAV viral particles are no more than one hour, two hours, three hours, four hours, five hours, six hours, nine hours, twelve hours or 24 hours apart.

In some embodiments, the invention provides a method for treating a human with MLD by administering an effective amount of a pharmaceutical composition comprising a recombinant viral vector encoding an ARSA polypeptide of the present disclosure. In some embodiments, the pharmaceutical composition comprises one or more pharmaceutically acceptable excipients.

In some embodiments, the methods comprise administering an effective amount of a pharmaceutical composition comprising a recombinant viral vector encoding an ARSA polypeptide of the present disclosure to treat MLD in an individual in need thereof. In some embodiments, the viral titer of the viral particles (e.g., rAAV particles) is at least about any of 5×10¹², 6×10¹², 7×10¹², 8×10¹², 9×10¹², 10×10¹², 11×10¹², 15×10¹², 20×10¹², 25×10¹², 30×10¹², or 50×10¹² genome copies/mL. In some embodiments, the viral titer of the viral particles (e.g., rAAV particles) is about any of 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some embodiments, the viral titer of the viral particles (e.g., rAAV particles) is about any of 5×10¹² to 10×10¹², 10×10¹² to 25×10¹², or 25×10¹² to 50×10¹² genome copies/mL. In some embodiments, the viral titer of the viral particles (e.g., rAAV particles) is at least about any of 5×10⁹, 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some embodiments, the viral titer of the viral particles (e.g., rAAV particles) is about any of 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹ or 50×10⁹ to 100×10⁹ transducing units/mL. In some embodiments, the viral titer of the viral particles (e.g., rAAV particles) is about any of 5×10⁹ to 10×10⁹, 10×10⁹ to 15×10⁹, 15×10⁹ to 25×10⁹, or 25×10⁹ to 50×10⁹ transducing units/mL. In some embodiments, the viral titer of the viral particles (e.g., rAAV particles) is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL. In some embodiments, the viral titer of the viral particles (e.g., rAAV particles) is at least any of about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL. In some embodiments, the viral titer of the viral particles (e.g., rAAV particles) is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL. In some embodiments, the viral particles are rAAV particles. In some embodiments, the rAAV particles comprise an AAV1999 capsid protein.

In some embodiments, the dose of viral particles administered to the individual is at least about any of 1×10⁸ to about 6×10¹³ genome copies/kg of body weight. In some embodiments, the dose of viral particles administered to the individual is about any of 1×10⁸ to about 6×10¹³ genome copies/kg of body weight. In some embodiments, the dose of viral particles administered to the individual is about any of 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 13×10¹², 4×10¹², 5×10¹², 6×10¹², 7×10¹², 8×10¹², 9×10¹², or 1×10¹³ genome copies/kg of body weight.

In some embodiments, the total amount of viral particles administered to the individual is at least about any of 1×10⁹ to about 1×10¹⁴ genome copies. In some embodiments, the total amount of viral particles administered to the individual is about any of 1×10⁹ to about 1×10¹⁴ genome copies. In some embodiments, the total amount of viral particles administered to the individual is about any of 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 13×10¹³, 4×10¹³, 5×10¹³, 6×10¹³, 7×10¹³, 8×10¹³, 9×10¹³, or 1×10¹⁴ genome copies.

Compositions of the invention (e.g., recombinant viral particles comprising a vector encoding an ARSA polypeptide of the present disclosure) can be used either alone or in combination with one or more additional therapeutic agents for treating MLD. The interval between sequential administration can be in terms of at least (or, alternatively, less than) minutes, hours, or days.

An effective amount of rAAV (in some embodiments in the form of particles) is administered, depending on the objectives of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. The rAAV composition may be administered by one or more administrations, either during the same procedure or spaced apart by days, weeks, months, or years. In some embodiments, multiple vectors may be used to treat the mammal (e.g., a human).

In some embodiments, a rAAV composition of the present disclosure may be used for administration to a human. In some embodiments, a rAAV composition of the present disclosure may be used for pediatric administration. In some embodiments, an effective amount of rAAV (in some embodiments in the form of particles) is administered to a patient that is less than one month, less than two months, less than three months, less than four months, less than five months, less than six months, less than seven months, less than eight months, less than nine months, less than ten months, less than eleven months, less than one year, less than 13 months, less than 14 months, less than 15 months, less than 16 months, less than 17 months, less than 18 months, less than 19 months, less than 20 months, less than 21 months, less than 22 months, less than two years, less than three years old, less than five years old or less than seven years old.

In some embodiments, a rAAV composition of the present disclosure may be used for administration to a young adult. In some embodiments, an effective amount of rAAV (in some embodiments in the form of particles) is administered to a patient that is less than 12 years old, less than 13 years old, less than 14 years old, less than 15 years old, less than 16 years old, less than 17 years old, less than 18 years old, less than 19 years old, less than 20 years old, less than 21 years old, less than 22 years old, less than 23 years old, less than 24 years old, or less than 25 years old.

### Kits or Articles of Manufacture

The expression cassettes (e.g., an expression cassette for expressing an ARSA polypeptide, such as a wild type human ARSA polypeptide), rAAV vectors, particles, and/or pharmaceutical compositions as described herein may be contained within a kit or article of manufacture, e.g., designed for use in one of the methods of the invention as described herein.

Generally, the system comprises a cannula, one or more syringes (e.g., 1, 2, 3, 4 or more), and one or more fluids (e.g., 1, 2, 3, 4 or more) suitable for use in the methods of the invention.

The syringe may be any suitable syringe, provided it is capable of being connected to the cannula for delivery of a fluid. In some embodiments, the system has one syringe. In some embodiments, the system has two syringes. In some embodiments, the system has three syringes. In some embodiments, the system has four or more syringes. The fluids suitable for use in the methods of the invention include those described herein, for example, one or more fluids each comprising an effective amount of one or more vectors as described herein, and one or more fluids comprising one or more therapeutic agents.

In some embodiments, the kit comprises a single fluid (e.g., a pharmaceutically acceptable fluid comprising an effective amount of the vector). In some embodiments, the kit comprises 2 fluids. In some embodiments, the kit comprises 3 fluids. In some embodiments, the kit comprises 4 or more fluids. A fluid may include a diluent, buffer, excipient, or any other liquid described herein or known in the art suitable for delivering, diluting, stabilizing, buffering, or otherwise transporting an expression cassette for expressing an ARSA polypeptide or rAAV vector composition of the present disclosure. In some embodiments, the kit comprises one or more buffers, e.g., an aqueous pH buffered solution. Examples of buffers may include without limitation phosphate, citrate, Tris, HEPES, and other organic acid buffers.

In some embodiments, the kit comprises a container. Suitable containers may include, e.g., vials, bags, syringes, and bottles. The container may be made of one or more of a material such as glass, metal, or plastic. In some embodiments, the container is used to hold a rAAV composition of the present disclosure. In some embodiments, the container may also hold a fluid and/or other therapeutic agent.

In some embodiments, the kit comprises an additional therapeutic agent with a rAAV composition of the present disclosure. In some embodiments, the rAAV composition and the additional therapeutic agent may be mixed. In some embodiments, the rAAV composition and the additional therapeutic agent may be kept separate. In some embodiments, the rAAV composition and the additional therapeutic agent may be in the same container. In some embodiments, the rAAV composition and the additional therapeutic agent may be in different containers. In some embodiments, the rAAV composition and the additional therapeutic agent may be administered simultaneously. In some embodiments, the rAAV composition and the additional therapeutic agent may be administered on the same day. In some embodiments, the rAAV composition may be administered within one day, two days, three days, four days, five days, six days, seven days, two weeks, three weeks, four weeks, two months, three months, four months, five months, or six months of administration of the additional therapeutic agent.

In some embodiments, the kit comprises a therapeutic agent to transiently suppress the immune system prior to AAV administration. In some embodiments, patients are transiently immune suppressed shortly before and after injection of the virus to inhibit the T cell response to the AAV particles (e.g., see Ferreira et al., Hum. Gene Ther. 25:180-188, 2014). In some embodiments, the kit further provides cyclosporine, mycophenolate mofetil, and/or methylprednisolone.

The rAAV particles and/or compositions of the invention may further be packaged into kits including instructions for use. In some embodiments, the kits further comprise a device for delivery (e.g., any type of parenteral administration described herein) of compositions of rAAV particles. In some embodiments, the instructions for use include instructions according to one of the methods described herein. In some embodiments, the instructions are printed on a label provided with (e.g., affixed to) a container. In some embodiments, the instructions for use include instructions for administering to an individual (e.g., a human) an effective amount of rAAV particles, e.g., for treating MLD in an individual.

### EXAMPLES

The invention will be more fully understood by reference to the following examples. They should not, however be construed as limiting the scope of the invention. It is understood that the examples and embodiments described herein are for illustrative purposes only and that various modification or changes in light thereof will be suggested to persons skilled in the art and are to be included within the spirit and purview of this application and scope of the appended embodiments.

### General Methods

### Tissue Homogenization

Tissues were homogenized with added cold 10 mM Tris 1 mM EDTA buffer in 1.4 mm ceramic bead homogenization tubes in refrigerated Omni bead ruptor—20 second cycle 4.7 oscillation/sec. Following homogenization, aliquots made for subsequent assay.

### Tissue Solubilization for Sulfatase Activity

To homogenized tissue, Nonidet P-40 was added to 0.1% final concentration, allowed to solubilize at 4C for 1.5 hours on an orbital shaker, then centrifuged at 18,000×g for 20 minutes. Supernatant removed and transferred to Eppendorf tube on ice.

### BCA Assay

Total protein concentration determined by BCA (bicinchoninic acid) assay (Thermo Scientific 23227) using 10 ul supernatant diluted in water. Colorimetric detection of the cuprous cation (Cu¹⁺) by bicinchoninic acid (BCA) by absorbance at 562 nm. Molecular Devices SpectraMax 340PC-384 with SoftMax Pro version 5.4.4 software used to read 96 well microliter plate.

### Sulfatase Activity

Ten microliters of clarified supernatant was assayed for total sulfatase activity using sulfatase activity assay for hydrolyzed 4-Nitrocatechol (PNC) from 4-Nitrocatechol Sulfate (PNCS) substrate. (Abecm Ab20473 1). Activity determined by hydrolyzed 4-Nitrocatechol (PNC) of sample relative to PNC standard curve and read absorbance at 515 nm. Sulfatase activity reported as mU/mg (nmol/min/mg). Molecular Devices SpectraMax 340PC-384 with SoftMax Pro version 5.4.4 software used to read 96 well microtiter plate.

### Lipid Extraction

An aliquot of tissue homogenate (or fluid) was extracted with 20-100× extraction solution (5 mM Ammonium Formate, 0.2% Formic Acid in Acetonitrile:Methanol (70:30), supplemented with 10 ng/ml C17-sulfatide). After mixing vigorously for 10 min, samples were sit for 5 min and vortexed again quickly (30 s). All samples were centrifuged at 8,400 rpm for 10 min at 4° C. An aliquot of the supernatant (200 uL) was transferred into a deactivated Q-sert Vial for LC-MS analysis.

Standard curve (linearity range 0.03-1000 ng/mL, 2×-serial dilutions) were prepared in the same extraction solution using sulfatides with C16, C18, C24 and C24:1chain length (Matreya) and C18 2R—OH sulfatide and lyso-sulfatide (Avanti).

### LC-MS Analysis of Sulfatides

A Waters Acquity UPLC system (Milford, MA) was coupled to a Qtrap 6500 mass spectrometer system (Framingham, MA) equipped with a ESI source operated in negative ion mode with the following parameters: curtain gas 25.0; ionSpray voltage −4.5 kV; temperature 500° C.; ion source gas: 50 and 70; declustering potential −80V; entrance potential -10V; collision energy −155V; and collision cell exit potential −15V.

The sulfatide species were separated on a Waters Acquity UPLC BEH Amide (1.7 μ, 2.1×100 mm, P/N: 186004801). The autosampler and column oven were maintained at 10° C. and 20° C., respectively. The mobile phase consisted of Solvent A: 5 mM Ammonium Formate in 95:5 of Acetonitrile:Water, and Solvent B: 5 mM Ammonium Formate in 90:10 Methanol:Water.

The gradient program started at 0% B and hold for 2 min, followed a linear curve from 0% B to 100% B. After 1 more min at 100% B, it re-equilibrated at 0% B. All sulfatide species are normalized to the internal standard of C17-sulfatide, and calculated by the standard curve of the corresponding standard sulfatides.

### Nitroblue Myelin Stain

Transfer sections to 12-well plate and perform all steps in free-floating sections. Wash section 2×1 min in TBA on shaker. Incubate in Nitroblue solution (Sigma cat #N5374) for 1 hour and rinse 2×5 min in TBA on shaker. Incubate sections in DAB stain (Sigma Kit cat #D4293) for 1 hour and rinse 2×1 in RO water. Rinse sections in PBS for 1 min, mount in PBS and air dry slides. Counterstain with Neutral Red solution for 2 min, wash 2×2 in RO water. Rinse slides for 1 min in 95% reagent alcohol, 2>2 min in 100% reagent alcohol, 2×2 min in Xylene, coverslip using Acrytol mounting medium.

### GFP ELISA

Measurement of GFP protein was completed using the GFP SimpleStep ELISA® Kit from Abcam (ab171581). It is imperative to read the protocol insert provided with each kit lot as some reagent concentrations change with different lots, particularly the standard.

Prior to beginning, all kit components were equilibrated to room temperature (18-25 C), at least 30 minutes. An aliquot of protein-processed tissue homogenate was thawed on ice. Reagents and working standards were prepared fresh, as per kit instructions. Samples were diluted in complete cell extraction buffer as follows: Grey Matter 1:5, Spinal Cord 1:5, Heart: 1:5, liver 1:20. Diluted sample and standard were added to the appropriate wells, followed by antibody cocktail to each well. The plate was sealed, incubated at room temperature for 1 hour, shaking at 400 RPM. Wells were washed 3 times with wash buffer, and complete removal of liquid was ensured. TMB solution was added to each well and incubated in the dark for 10 minutes at room temperature, shaking at 400 RPM. Quickly after TMB incubation, stop solution was added to each well and mixed on a plate shaker for 1 minute. The plate was read at 450nm on a SpectraMax plate reader (Molecular Devices) using Softmax software.

### FLAG IHC

The NHP brain FFPE slides were treated in EDTA solution (pH. 9.0) for 20 minutes at 90° C. for antigen retrieval, followed by blocking with 3% hydrogen peroxide for 5 minutes. The slides were then incubated with Flag DDK antibody (Abcam ab205606) at 1:200 dilution in antibody diluent (Cell Signaling #8112) for one hour at room temperature followed by anti-rabbit HRP (Abcam ab6721) secondary antibody incubation. The color development for Flag DDK signal was achieved by incubating slides in DAB solution for 3 minutes at room temperature, and the slides were subsequently counter stained with hematoxylin for nuclei staining.

### GFP IHC

The NHP brain FFPE slides were treated in EDTA solution (pH. 9.0) for 20 minutes at 90° C. for antigen retrieval and subsequently blocked with 3% hydrogen peroxide for 10 minutes, followed with 5% horse serum for 45 minutes at room temperature. The slides were then incubated with GFP antibody (ThermoFisher A-11122) at 1:500 dilution in antibody diluent (Cell Signaling #8112) for one hour at room temperature followed with anti-Rabbit HRP (Abcam ab6721) secondary antibody incubation. The color development for GFP signal was achieved by incubating slides in DAB solution for 3 minutes at room temperature, and the slides were counter stained with hematoxylin for nuclei staining.

### Cross Correction Analysis

Adjacent 5 μm FFPE sections were processed for either ARSA IHC and DAPI or WPRE ISH and DAPI. Individual tiles were then stitched to obtain sagittal sections for both IHC and ISH images. These images were then registered globally with a transformation matrix (rotational and translational parameters) to align the IHC and ISH images. Individual tiles within the sagittal sections were then registered locally using DAPI to ensure good alignment between individual IHC and ISH tiles. In each tile, IHC positive cells and ISH positive cells were estimated by thresholding parameters empirically determined across the entire sagittal sections. In the case ISH, IHC positive cells had to have at least 20% overlap with a nuclei to be counted as a bonafide ISH signal. For IHC images, negative controls with no ARSA staining were used to determine thresholding parameters. Cross-correction factor which is the ratio of IHC positive cells to ISH positive cells was estimated for each tile and represented as a heat map.

### Single-Nuclei Sequencing

Nuclei Isolation: For nuclei isolation, dissected brains were transferred to microcentrifuge tubes, snap frozen in a slurry of dry ice and ethanol, and stored at −800 C until the time of use. To isolate nuclei, frozen mouse brains were put into nuclei lysis buffer containing 0.1% Triton-100 (Sigma-Aldrich), 1 mM DTT (Sigma-Aldrich(, and 0.2 U/ul RNase Inhibitor (Sigma-Aldrich) in 1 ml Dounce Homogenizer (Wheaton). Tissue was homogenized using 10 strokes of the loose Dounce pestle followed by 10 strokes of the tight pestle, and incubated on ice for 15 min. The resulting homogenate was passed through 301.tm cell strainer (Miltenyi Biotech) and centrifuged at 500×g for 5 min to pellet nuclei. Nuclei were resuspended in buffer containing 1×PBS (Thermo Fisher), 1% nuclease-free BSA(Sigma-Aldrich), 1 mM DTT (Sigma-Aldrich) and 0.2 U/ul RNase inhibitor (Sigma-Aldrich). Mouse anti-NeuN conjugated to PE (EMD Millipore) was added to preparations at a dilution of 1:500 and samples were incubated for 30 min at 40 C. Samples were then centrifuged for 5 min at 500×g to pellet nuclei, and pellets were resuspended in 1×PBS, 1% BSA, 1 mM DTT, and 0.2 U/ul RNase inhibitor. DAPI was added at a concentration of 0.1 ug/ml. Single nuclei sorting was carried out on Influx-83 (BD Biosciences) using 100 μm nozzle. Nuclei were gated on DAPI and NeuN signal (PE).

### Library Preparation and NovaSeq Sequencing

Libraries were prepared according to 10× Genomics protocol for Chromium Single Cell 3′ Gene Expression V3.1 kit. Briefly, immediately after sorting, the nuclei were mixed with 90% NeuN− and 10% NeuN+ nuclei. The GEM generation step in 10× Genomics gene expression kit (10× Genomics) was carried out. After GEM generation, samples were kept at −20° C. for further step. All samples were processed together from cDNA amplification, library construction, to libraries sequencing. The quantification of cDNA and libraries were solid for all samples. The libraries were sequenced on illumine Novaseq 6000. Libraries were sequenced at a median depth of around 50K reads/nuclei. UMI count matrices generated by Cell Ranger V5.

Data Preprocessing: The generated count matrices together with nucleus barcodes and gene labels were loaded onto Partek Flow. For Quality Control (QC), nuclei were filtered following standard protocols based on examination of violin plots. The detailed cutoffs were 250<nFeature_RNA<6000 and nCount RNA<30000. The mitochondria protein coding genes were <2%. After quality filtering, 147313 total nuclei remained for the further analysis.

### Vector Genome Assessment

HT gDNA isolation: The gDNA was isolated from 50 ul NHP tissue homogenate using QIAmp 96 DNA QlAcube HT kit (cat #51331) according to manufacturer's protocol “QIAamp® 96 DNA QlAcube® HT Handbook” The tissue homogenate was treated with proteinase K at 56 C overnight first, then transferred to S block and place the samples into QIAcube HT instrument performing gDNA isolation with the QIAcube HT Prep Mange Softer ware. gDNA concentration then was measured with NANODROP 8000 (Thermo Fisher Scientific).

dPCR via QIAcuty: Vector genome was determined with dPCR via QIAcuty that manufactured by QIAGEN. 7 ug of gDNA isolated from NHP tissue and 5 ul of master reaction mix which contain 1× probe PCR Master Mix (cat #250103); 1× primer-probe mix1 (BGH) and mix2 (housekeep gene); 0.25 U of HindIII restriction enzyme was mixed in standard PCR plate and then transferred to nanoplate. Total reaction volume is 12 ul per well. Then place the nanoplate into QIAcuity instrument and perform dPCR use manufacture suggested cycling. DNA copy for each sample and each gene was analyzed automatically by the softer ware. Then vector genome per cell was calculated as: 2×BGH copies divide housekeep gene copies.

### Example 1. Arsa Expression In Vivo with Two Different Expression Cassettes

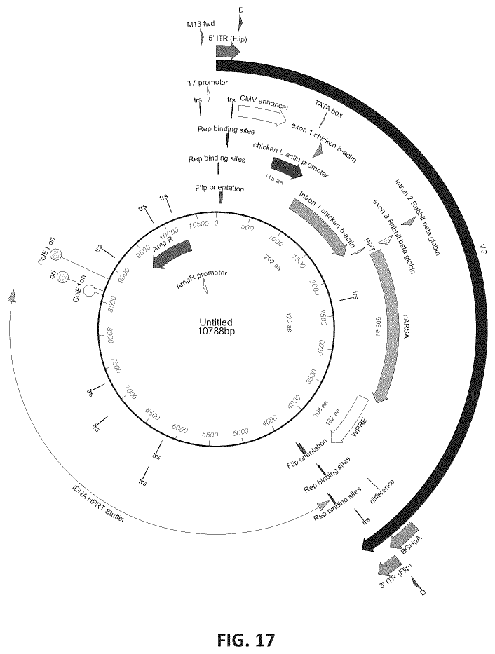

The vector payload was designed to express the human codon optimized arylsulfatase A (ARSA) gene (SEQ ID NO: 2) driven by the constitutive chimeric CMV-chicken β-actin (CBA) promoter. Transgene expression is further enhanced with the addition of the woodchuck hepatitis virus post-transcriptional regulatory element (WPRE). SEQ ID NO: 3 shows a representative full DNA sequence of plasmid for rAAV packaging, including 5′ AAV2 ITR (SEQ ID NO: 4) and 3′ AAV2 ITR (SEQ ID NO: 5), CMV enhancer element (SEQ ID NO: 6), chicken β-actin promoter (SEQ ID NO: 7) and WPRE element (SEQ ID NO: 8). A schematic of a map of a particular plasmid for rAAV particle production is shown in FIG. 17. Particular elements of the plasmid are listed in Table 1.

TABLE 1
Sequence Annotation from 5′ITR to 3′ITR

| Sequence | |
|---|---|
| Numbering | Annotation |
| 1-145 | AAV2 5′ ITR |
| 173-553 | CMV enhancer element |
| 554-873 | Chicken b-actin promoter |
| 874-924 | Exon 1 chicken b-actin |
| 925-1799 | Intron 1 chicken b-actin |
| 1808-1848 | Intron 2 rabbit beta globin |
| 1849-1902 | Exon 3 rabbit beta globin |
| 1914-3443 | Codon optimized human ARSA |
| 3446-4030 | WPRE element |
| 4043-4245 | polyA signal sequence; Bovine growth |
| | hormone gene polyadenylation sequence |
| 4253-4397 | AAV2 3 ITR element in flip orientation |

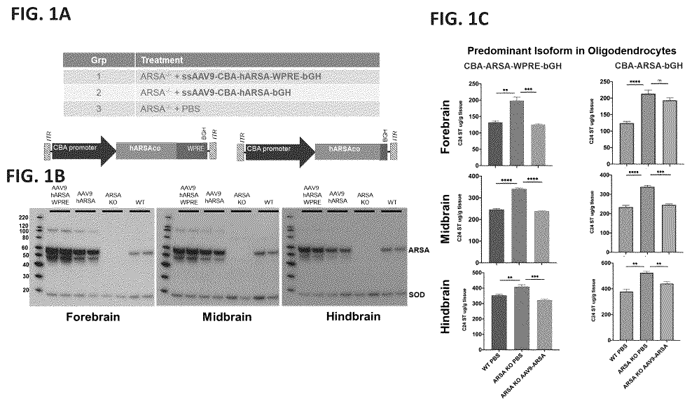

Post-natal day two ARSA^{-/-} mice were administered IV (bilateral retro-orbital injection) with PBS or AAV9 (2e11 VGs; 2e14 VG/kg) rAAV viral particles expressing human ARSA with or without the WPRE element. The two expression cassettes are schematized in FIG. 1A. FIG. 1B shows a Western blot analysis of hARSA expression in fore-, mid- and hindbrain following administration of the viral particles. The expression levels are compared to expression levels in ARSA^{-/-} mice and mice expressing normal levels of ARSA (i.e., WT mice).

As shown in FIG. 1B, expression levels are increased following administration of the viral particles comprising the two expression cassettes. Inclusion of the WPRE element in the expression cassette gives increased expression levels of ARSA relative to the expression cassette without the WPRE element.

Next, LC-MS was used to measure C24-ST levels. ST levels were normalized to tissue weight. FIG. 1C shows CST levels in the forebrain, midbrain and hindbrain following administration of the viral particles comprising the two expression cassettes. Error bars represent mean with standard deviation. One-way ANOVA with Tukey's multiple comparison test. *p<0.05; **p<0.01; ***p<0.001; ****p<0.0001. ELN: 20200205-188, 20200305-152, 20200205-006. C24-ST levels following administration of AAV9 particles to ARSA^{-/-} mice comprising expression cassettes without the WPRE element were significantly decreased relative to C24-ST levels in ARSA^{-/-} mice. Even further decreases in C24-ST levels were observed following administration of AAV9 particles to ARSA^{-/-} mice comprising expression cassettes with the WPRE element.

### Example 2. Evaluation of Sulfatase Activity In ARSA^{-/-} Mice Dosed with AAV.RH10-CBA-ARSA-WPRE

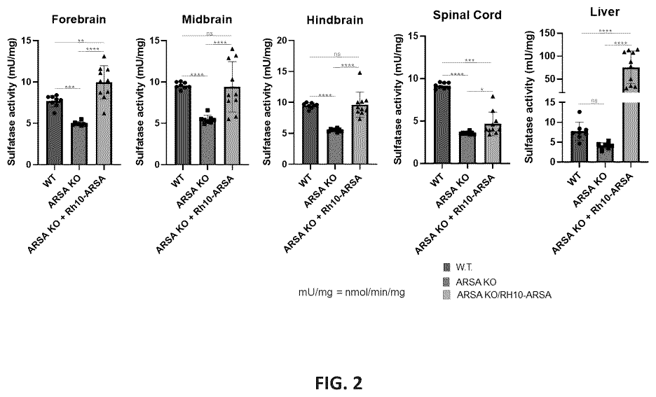

Late-stage (13 month) ARSA^{-/-} mice were dosed with AAV.rh10-CBA-ARSA-WPRE (1.97e11 VGs per animal). As controls, age-matched WT and ARSA^{-/-} mice were injected with formulation buffer. Three months post-dosing, mice were euthanized, and the brain, spinal cord and liver were collected. ARSA-mediated sulfatase activity was measured using the Sulfatase Activity Assay Kit (ab204731); data normalized to total protein measured by BCA assay. Error bars represent mean with standard deviation. Two-way ANOVA with Tukey's multiple comparison test. *p<0.05; **p<0.01; ***p<0.001; ****p<0.0001. ELN: 20201205-001, 20210225-107, 20210211-117. As shown in FIG. 2, AAV.rh10-CBA-ARSA-WPRE treated late-stage MLD (ARSA^{-/-}) mice show increased ARSA-mediated sulfatase activity in brain, spinal cord and liver.

### Example 3. Evaluation of Sulfatide in ARSA^{-/-} Mice Dosed with AAV.RH10-CBA-ARSA-WPRE

Sulfatide deposition in MLD patients is the primary driver of toxicity and neuronal death. Short-chain fatty acids (C16, C18) predominantly accumulate in neurons and astrocytes while long-chain fatty acid sulfatides (C24:1, C24) accumulate in myelin forming cells. Lyso-sulfatide, the deacylated form of sulfatides, accumulate in all tissues of MLD patients.

Late-stage (13 month) ARSA^{-/-} (ARSA KO) mice were dosed with AAV.rh10-CBA-ARSA-WPRE. As controls, age-matched WT and ARSA^{-/-} were injected with formulation buffer. Three months post-dosing, mice were euthanized, and the brain and spinal cord were collected. Sulfatide levels were measured using LC-MS. Data normalized to tissue weight: converted from ng/mL (50 μL) to μg/g. Error bars represent mean with standard deviation. Two-way ANOVA with Tukey's multiple comparison test.

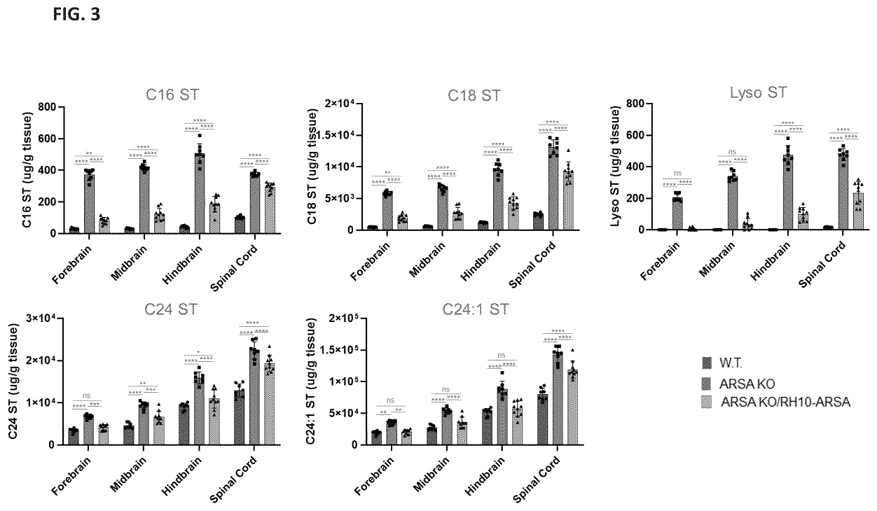

Sulfatide levels measured using LC-MS and both brain and spinal cord samples in ARSA^{-/-} mice dosed with AAV.rh10-CBA-ARSA-WPRE showed a significant decrease in all sulfatide isoform levels (FIG. 3).

### Example 4. Evaluation of Myelination in ARSA^{-/-} Mice Dosed with AAV.RH10-CBA-ARSA-WPRE

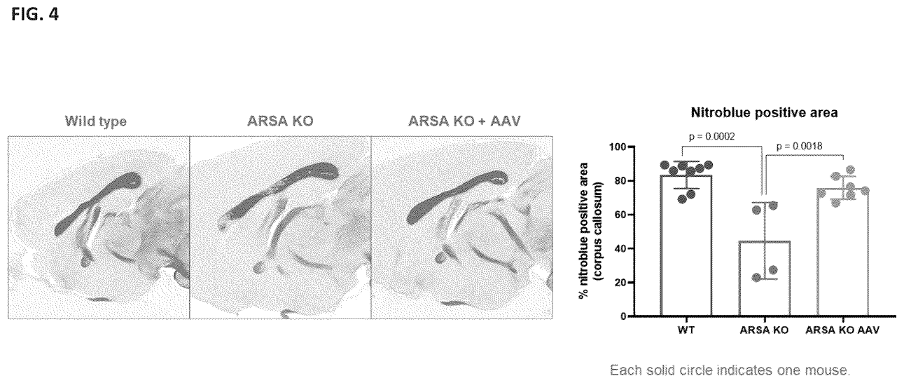

MLD patients exhibit significant loss of myelination in white matter tracts. To address the impact of gene therapy on late-stage (13 month) ARSA^{-/-} mice, mice were dosed with AAV.rh10-CBA-ARSA-WPRE. As controls, age-matched WT and ARSA^{-/-} were injected with formulation buffer. Three months post-dosing matched sagittal brain hemisections were stained with nitroblue and signal intensity quantitation performed specially in the corpus callosum. Error bars represent mean with standard deviation. One-way ANOVA with Tukey's multiple comparison test. AAV.rh10-CBA-ARSA-WPRE treated late-stage MLD (ARSA^{-/-}) mice show improved corpus callosum myelination relative to control (FIG. 4).

### Example 5. Evaluation of Myelin-Forming And Mature Oligodendrocyte Cell Numbers in ARSA^{-/-} Mice Dosed with AAV.RH10-CBA-ARSA-WPRE

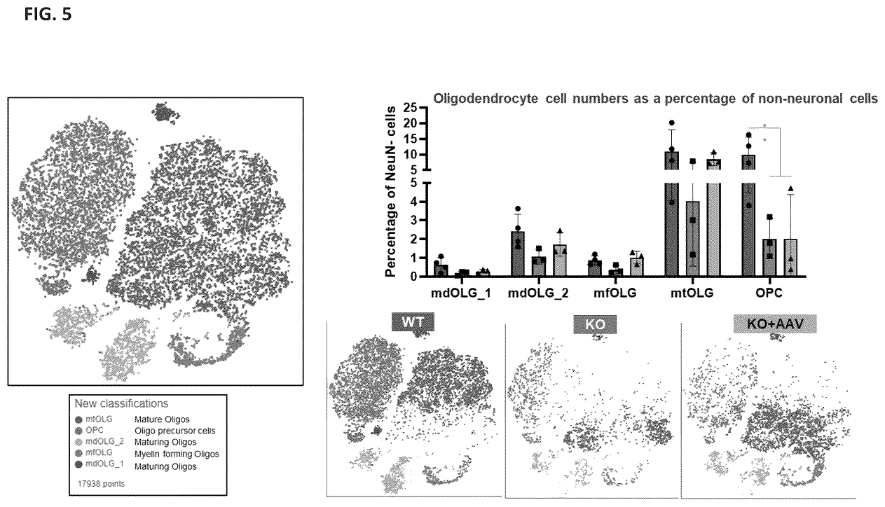

Late-stage (13 month) MLD ARSA^{-/-} mice were dosed with AAV.rh10-CBA-ARSA-WPRE. As controls, age-matched WT and ARSA^{-/-} mice were injected with formulation buffer. Brain hemispheres were collected flash frozen; the cerebellum, brain stem and olfactory bulb was dissected out. The rest of the brain was processed for single-nucleus RNAseq to measure relative abundance of different cell types. Error bars represent mean with standard deviation. Two-way ANOVA with Tukey's multiple comparison. FIG. 5 shows that AAV.rh10-CBA-ARSA-WPRE treated ARSA^{-/-} mice revealed a normalization in oligodendrocyte cells counts, as compared to untreated ARSA^{-/-} mice.

### Example 6. ARSA Protein Cross-Correction Studies

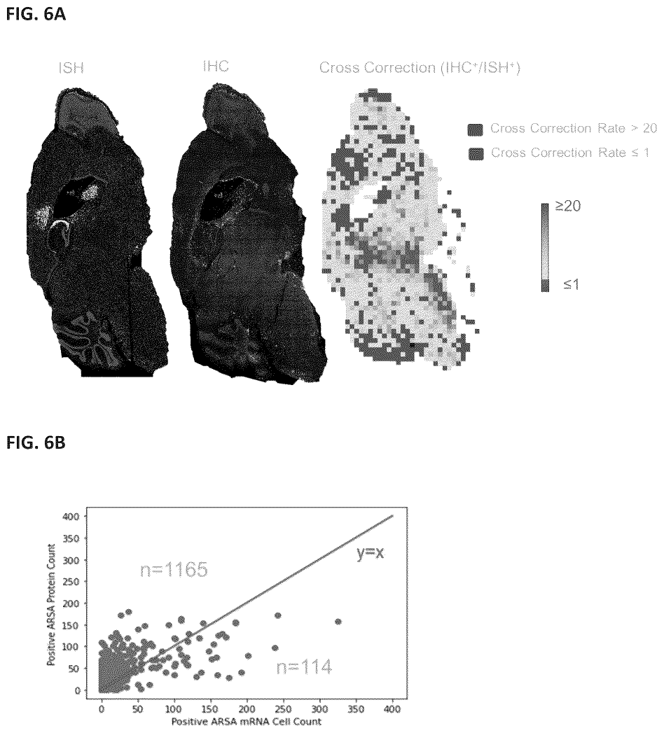

Late-stage (13 month) ARSA^{-/-} were dosed with AAV.rh10-CBA-ARSA-WPRE. Three months post-dosing, ARSA-mRNA in situ hybridization (ISH) and ARSA-protein immunohistochemistry (IHC) were performed on matched sagittal brain hemi-sections. The sections were imaged and analyzed for signal overlay. (FIG. 6A) Adjacent sagittal sections from mouse brain were treated for WPRE ISH or ARSA IHC with DAPI staining for nuclei. The sagittal sections were then analyzed as individual 1024×1024 pixel tiles. In each tile, ISH positive cells and IHC positive cells were determined by thresholding parameters empirically determined across the entire sagittal images. Cross-correction factor, which is the ratio of IHC+ cells to ISH+ cells is represented as a heat map with highly cross-corrected tiles shown in shades of red (FIG. 6B). ISH+ cell count vs IHC+ cell count from each tile is plotted as a scatter plot with y=x line shown in red. Tiles above the y=x line indicate cross-corrected cells. In late-neuropathic mice, 10 fold higher cells were cross corrected, compared to infected cells, highlighting the broad cross correction achieved with this strategy.

### Example 7. Evaluation of AAV.rh10-hARSA-WPRE Mediated Gene Replacement in MLD Mice (ARSA^{-/-}) at Early (6 Mo) Neuropathologic Stages Resulted in the Reversal of MLD-Associated Pathology

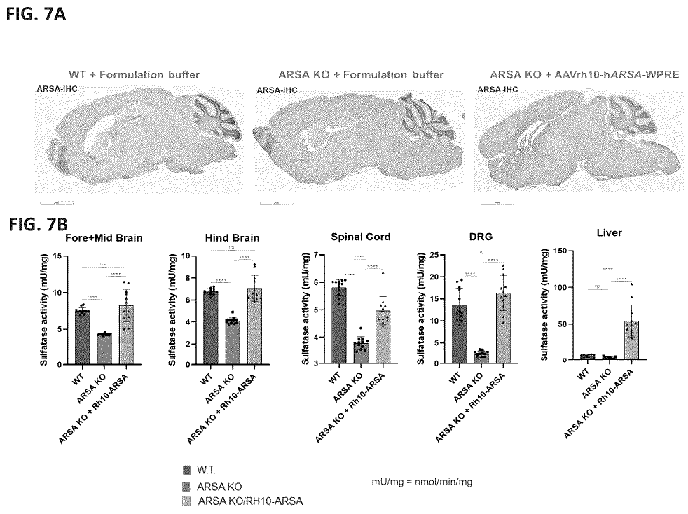

Early-stage (6 month) ARSA^{-/-} mice were dosed with AAV.rh10-CBA-ARSA-WPRE. As controls, age-matched WT and ARSA^{-/-} mice were injected with formulation buffer. Four months post-dosing, broad ARSA expression was observed in sagittal brain sections by IHC against human ARSA (FIG. 7A). In the same animals, brain, spinal cord, DRGs, and liver showed a significant increase in ARSA activity (FIG. 7B). Hence, AAV.rh10-CBA-ARSA-WPRE treated early-stage MLD (ARSA^{-/-}) mice show broad CNS human ARSA expression and increased ARSA-mediated sulfatase activity in brain, spinal cord, DRGs and liver.

### Example 8. Evaluation of Sulfatide Levels in AAV.rh10-hARSA-WPRE Mediated Gene Replacement in MLD Mice (ARSA^{-/-}) at Early (6 Mo) Neuropathologic Stages

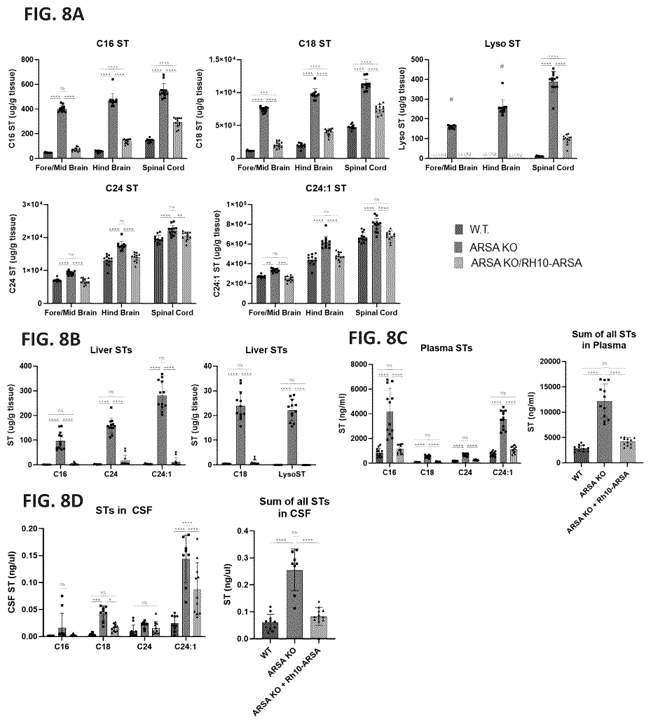

Early-stage (6 month) ARSA^{-/-} mice were dosed with AAV.rh10-CBA-ARSA-WPRE. As controls, age-matched WT and ARSA^{-/-} mice were injected with formulation buffer. Four months post-dosing, animals were euthanized, and (A) brain and spinal cord, (B) liver, (C) plasma and (D) CSF were collected. Sulfatide levels were measured using LC-MS. Data was normalized to tissue weight: converted from ng/mL (50 μL) to μg/g. Fluids: Data converted to (30 μL) ng/mL to ng/mL, and ng/mL (5 μL) to ng/uL. FIGS. 8A-D show sulfatide levels in brain (FIG. 8A), liver (FIG. 8B), plasma (FIG. 8C) and CSF (FIG. 8D). Sulfatide levels were increased significantly in the brain, spinal cord, liver, plasma, and CSF following administration of AAV.rh10-CBA-ARSA-WPRE.

### Example 9: Studies with Non-Human Primates (NHPS)—Sulfatase Activity

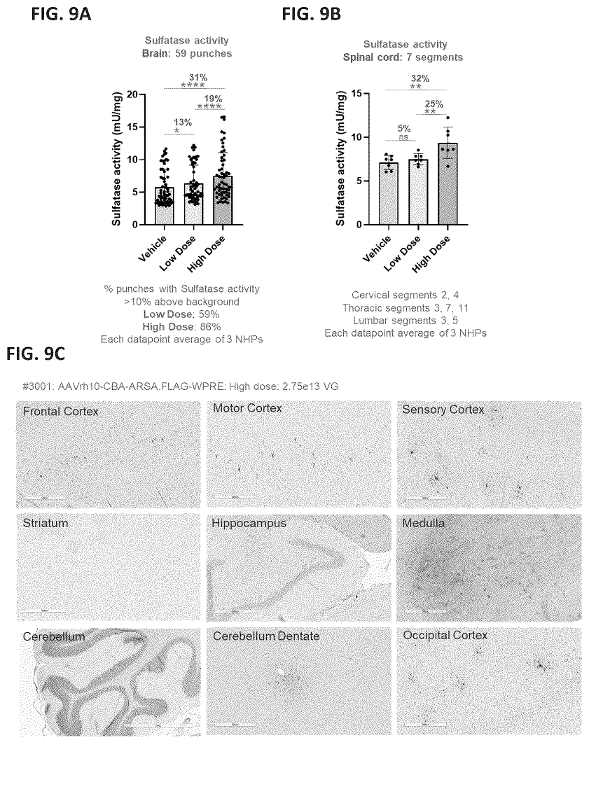

Cynomolgus monkeys (Male, Mauritian 2 yr old, 2-3 Kg) seronegative for AAVrh.10 were dosed AAV.rh10-CBA-ARSA-WPRE intrathecally at the cervical level 1-2 junction using a ported intrathecal catheter inserted at the lumbar region. Animals were dosed in the Trendelenburg position with two 2.5 mL infusions at 0.125 mL/min, approximately six hours apart. Two doses were administrated: 7.5e12 VG/NHP (@1e11 VG/brain gram) and 2.75e13 VG/NHP (@3.3e11 VG/brain gram). 29 days post-dosing, animals were euthanized and samples were assessed for ARSA activity: 59 tissues punched from brain and 7 tissue punches from spinal cord. Significantly increased sulfatase activity in the brain and spinal cord was observed with 59% and 86% of punches across the brain showing at least 10% increase activity over background at low and high doses, respectively (FIG. 9A). This exceeded our target criteria of 50% punches with >10% increase activity. Similarly, 25% and 32% increase in sulfatase activity was observed in spinal cord of treated NHPs at low and high doses, respectively (FIG. 9B). Broad ARSA biodistribution was observed in the brain of treated NHPs by IHC against the FLAG antibody (FIG. 9C). Our results indicate that AAV.rh10-CBA-ARSA-WPRE mediated gene replacement is a viable approach to achieve broad and therapeutic levels of ARSA in the central nervous system (CNS) and peripheral nervous system (PNS).

### Example 10: Studies with Non-Human Primates (NHPS)—Comparative Transgene Expression Levels Following Administration of Particles Comprising AAV1999 or AAV.rh10

Cynomolgus monkeys (Male, Mauritian 2 yr old, 2-3 kg) seronegative for AAVrh10 and AAV1999 (VP1 having SEQ ID NO. 12; VP2 having SEQ ID NO. 13; and VP3 having SEQ ID NO:14) were dosed intrathecally at the cervical level 1-2 junction using a ported intrathecal catheter inserted at the lumbar region. Animals were dosed in the Trendelenburg position with two 2.5 mL infusions at 0.125 mL/min, approximately 6 hours apart. One dose of AAV1999/rh10/Myo-CBA-eGFP was administrated at 2.75e13 VG/NHP (@3.3e11 VG/brain gram). Two weeks post-dosing, animals were euthanized, and samples were assessed for green fluorescent protein (GFP) expression by ELISA.

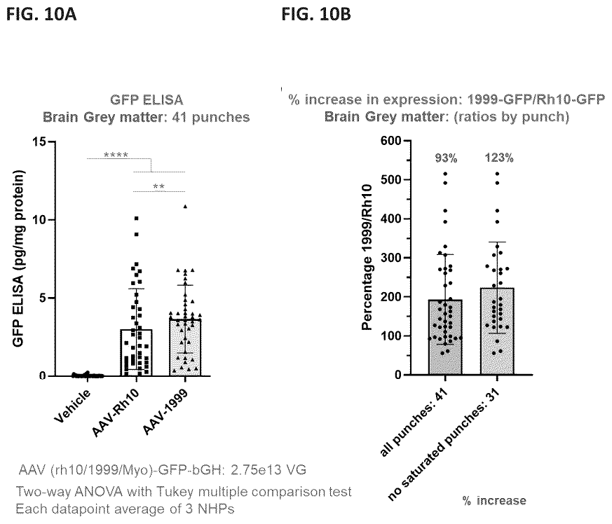

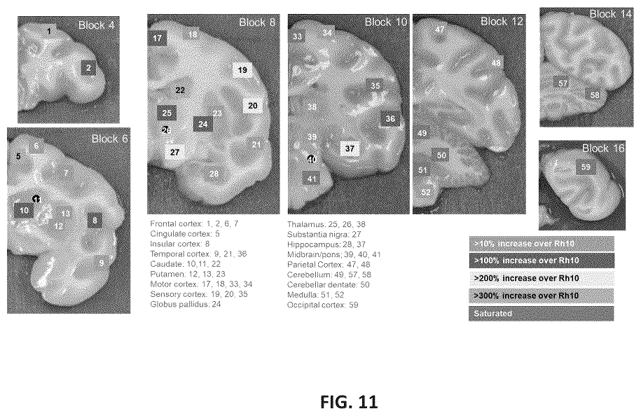

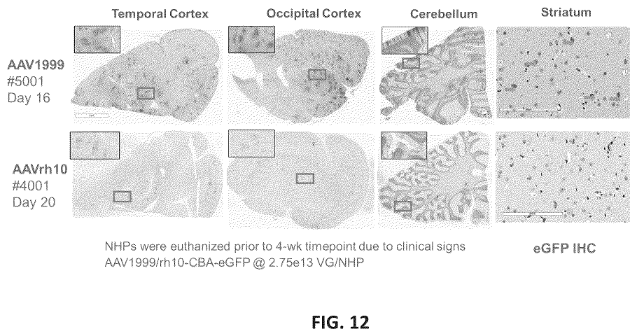

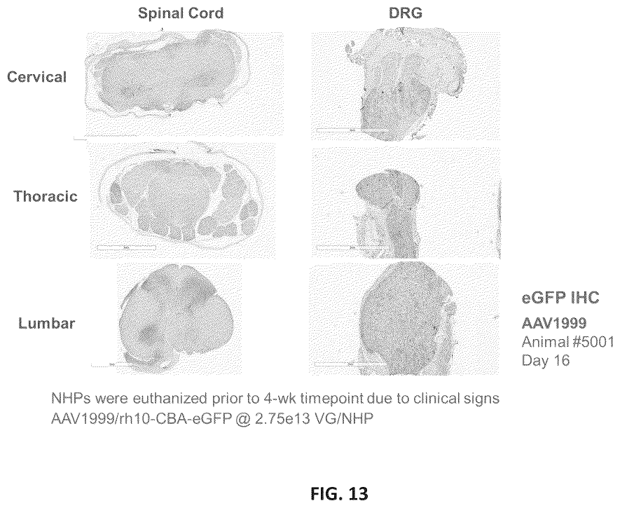

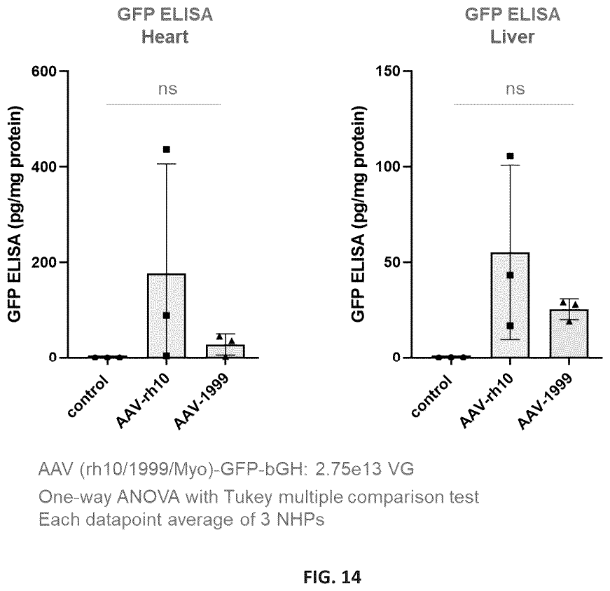

A total of 41 brain punches (grey matter) representing 16 different brain regions showed significantly higher GFP expression from AAV1999 as compared to AAVrh.10 (FIG. 10A). Across all samples, GFP expression in brain of AAV1999-treated NHPs was 93%-123% higher, as compared to AAVrh.10-treated NHPs (FIG. 10B). This is demonstrated by the heatmap, where in 38 out of 41 punches (averaged across three NHPs) in AAV1999 treated NHPs show at minimum 10% increase in GFP expression, compared to AAVrh10 treated NHPs (FIG. 11). Representative matched brain sections stained with antibodies at eGFP show robust expression in AAV1999 treated NHPs, demonstrating superior biodistribution compared to AAVrh10 (FIG. 12). Furthermore, in the spinal cord and DRGs, GFP expression in AAV1999-treated NHPs was 79% and 22% higher as compared to AAVrh.10-treated NHPs, respectively. FIG. 13 shows GFP expression in the spinal cord and DRGs of the NHPs. AAV1999-GFP expression in the heart and liver were lower, compared to AAVrh10-GFP treated NHPs (FIG. 14).

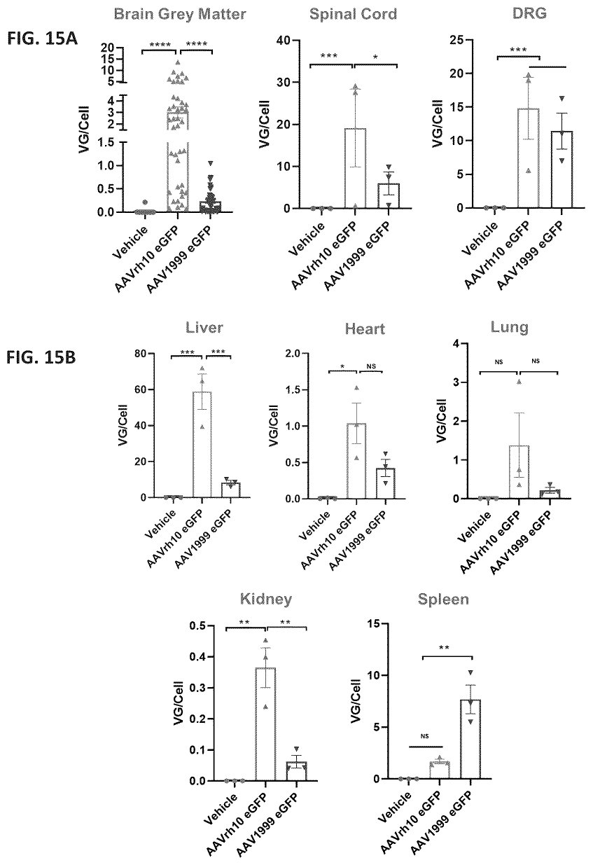

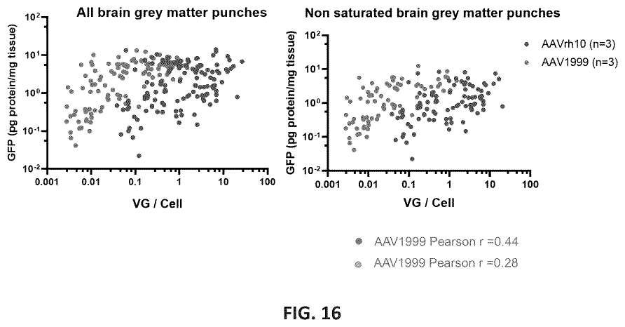

AAV1999 vector genome load was ten times lower in the brain and three times lower in the spinal cord, compared to AAVrh.10 (FIG. 15A). This is noticeable in the ‘left-shift’ in the correlation between GFP expression (y-axis) and tissue vector genomes (x-axis), where in the correlation for AAV1999 (red) is left shifted indication higher protein expression and lower (˜10 fold) tissue dose of vector (FIG. 16). Furthermore, DRGs, liver, heart, lung, and kidney also show lower AAV1999 vector genomes compared to AAVrh.10 (FIG. 15B). Spleen is the only tissue tested that shows higher AAV1999 vector genomes compared to AAVrh.10 (FIG. 15B). These results indicate that AAV1999 is superior to AAVrh.10 and gives broader biodistribution and higher transgene expression at a lower dose.

### Example 11: Long-Term Pharmacology, Efficacy and Durability Study in Pre-Neuropathic ARSA KO Mouse Model Using AAV1999-ARSA

This study was designed to evaluate long-term pharmacology and efficacy of AAV1999-ARSA in an Arsa KO mouse model. The study design is summarized in Table 2. Two-month-old Arsa KO mice were dosed (bilateral ICV injection; 4 uL per hemisphere) with AAV1999-ARSA (LOT #VP091321) at 1.6e11 VG per mouse (3.3e11 VG/gm brain weight). As controls, age-matched WT and Arsa KO mice were dosed with formulation buffer. Mice were euthanized 13 months post-dose and samples collected.

TABLE 2
Long-term pharmacology, efficacy and durability study in Arsa KO mice (2mo-15mo)

| | Number/ | | | | Dosing | Time points, sample |
|---|---|---|---|---|---|---|
| Group | sex | Genotype | Test article | Dose | regimen | collection, analysis |
| 1 | 12 (6 M, 6 F) | WT | Formulation buffer | N/A | Bi-lateral | [symbol] Dosing age: 2 mo |
| | | | | | ICV, 4 ul each | [symbol] Necropsy age: 15 mo |
| 2 | 12 (6 M, 6 F) | ARSA^{-/-} | Formulation buffer | N/A | side | (13 mo in-life) |
| 3 | 12 (6 M, 6 F) | ARSA^{-/-} | AAV1999-ARSA | 1.6e11VG | | [symbol] Brain, spinal cord, |
| | | | (LOT# VP091321) | (3.3e11VG/gram | | DRG, sciatic nerve, |
| | | | | brain weight) | | liver, CSF and plasma |

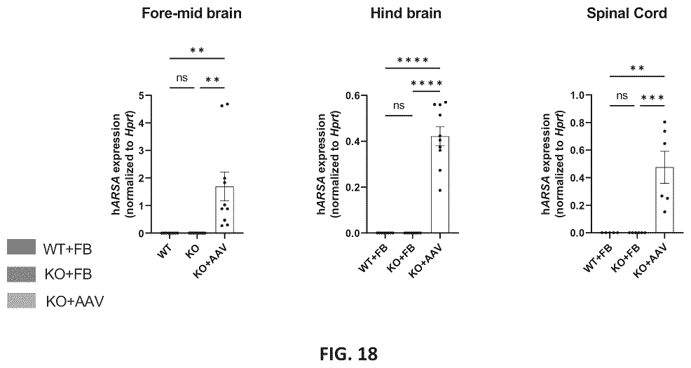

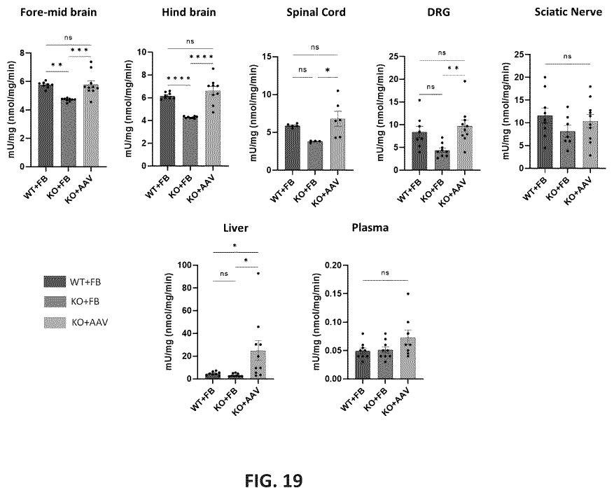

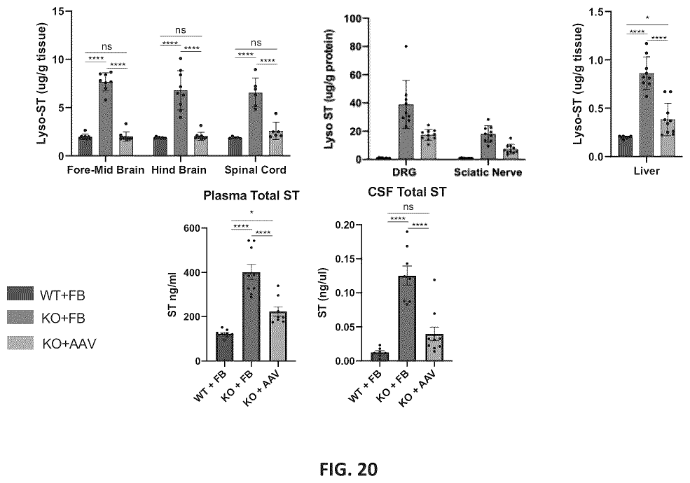

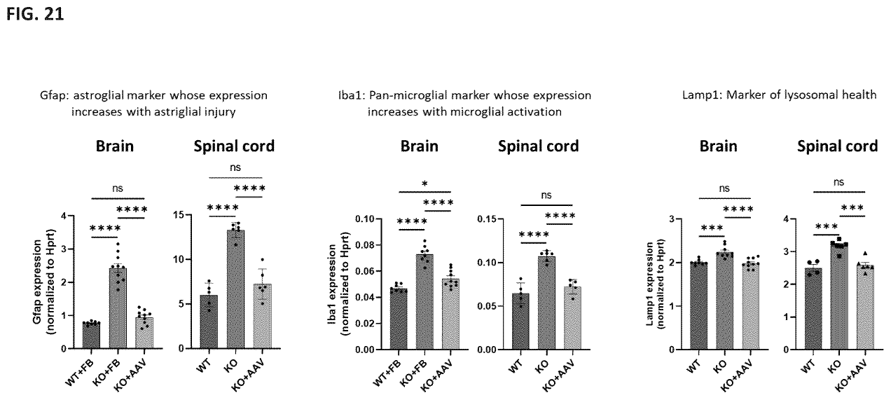

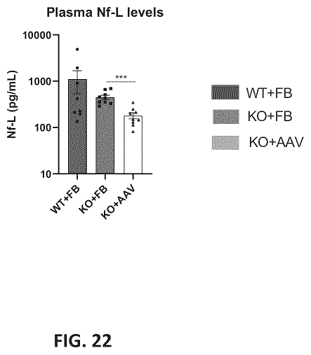

Robust hARSA mRNA expression was observed in the brain and spinal cord of AAV1999-ARSA treated Arsa KO mice (FIG. 18). Sulfatase activity was measured using the Sulfatase Activity Assay Kit (Abcam, ab204731). Arsa KO mice dosed with AAV1999-ARSA showed normal (equivalent to WT) levels of sulfatase activity in brain, spinal cord, DRG and sciatic nerve (FIG. 19). A measurable increase over WT sulfatase activity levels was observed in liver of treated mice (FIG. 19). Sulfatide deposition in MLD patients is the primary driver of toxicity and neuronal death. Lyso-sulfatide (lyso-ST), the deacylated form of sulfatide, accumulates in all tissues and is a characteristic hallmark of MLD-associated pathology in patients. In AAV1999-ARSA treated Arsa KO mice, lyso-ST levels returned to normal (similar to WT) in the brain and spinal cord (FIG. 20). Significant reduction in lyso-ST levels were also observed in DRGs, sciatic nerve and the liver, while total sulfatide levels were significantly reduced in plasma and CSF from treated animals (FIG. 20). Consequent to sulfatide clearance in the brain and spinal cord, AAV1999-ARSA treated Arsa KO mice showed significant improvement in neuroinflammatory markers (Gfap and Aif1) and Lamp1 expression (marker of lysosomal health) in the brain and spinal cord (FIG. 21). Furthermore, a significant reduction in plasma Nf-L levels were observed in AAV1999-ARSA treated Arsa KO, compared buffer treated Arsa KO mice (FIG. 22).

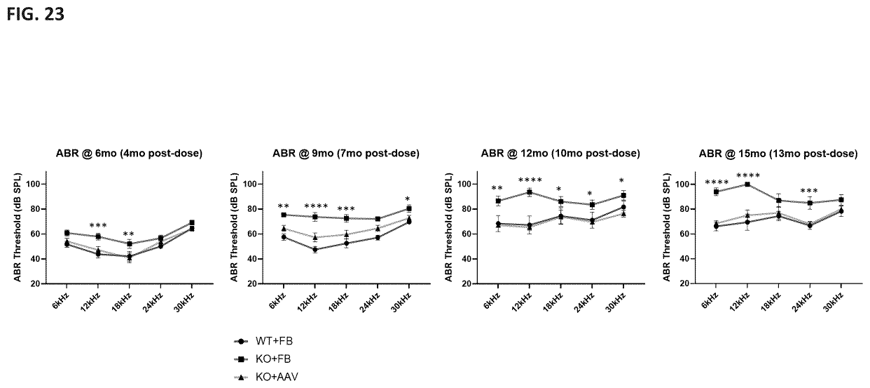

MLD patients often present with hearing impairment. The auditory brainstem response (ABR) test provides functional information about the inner ear (cochlea) and the central pathways for hearing. The ABR reflects the electrical responses of both the cochlear ganglion neurons and the nuclei of the central auditory pathway to sound stimulation (Zhou et al., 2006; Burkard et al., 2007). The ABR is recorded via electrodes placed on the scalp of an anesthetized animal. ABR thresholds refer to the lowest sound pressure level (SPL) that can generate identifiable electrical response waves. Arsa KO show progressive deterioration in hearing with time, which is reversed upon treatment with AAV1999-ARSA (FIG. 23). Partial reversal of phenotype is observed as early at 4 months post-dosing (earliest time post-dose tested) (FIG. 23).

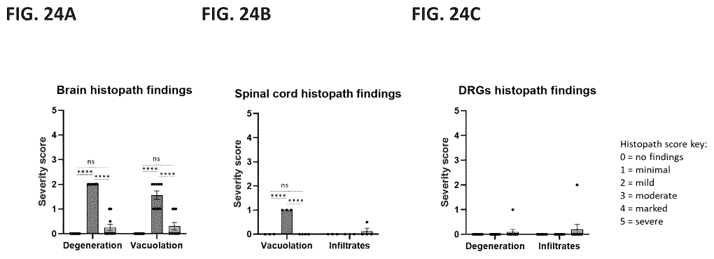

Vacuolation and neurodegeneration were observed in the brains of all Arsa KO mice usually in brainstem and/or cerebellum. In treated mice, histological changes were detected only in 4 of 10 mice, and the severity was lower than the Arsa KO mice (FIG. 24A). All Arsa KO mice showed vacuolation of minimal severity in the spinal cord, while no changes were detected in WT mice or AAV1999-ARSA treated Arsa KO mice (FIG. 24B). Finally, no pathological changes were detected in DRGs in any group (except 1 of 10 animals in KO+AAV group) (FIG. 24C). Therefore, treatment of Arsa KO mice with AAV1999-ARSA significantly minimized and/or prevented MLD-associated pathology in the brain and prevented MLD-associated pathology in the spinal cord.

### Example 12: Longitudinal Pharmacology, Efficacy and Durability Study in Early-Neuropathic ARSA KO Mouse Model Using AAV1999

This study was designed to evaluate target engagement and reversal of MLD-associated pathological and biochemical phenotypes in the Arsa KO model over a 6-month period. The early-neuropathic stage (6mo at dose) was selected as its most relevant to the targeted patient population. The study design is summarized in Table 3. Six-month-old Arsa KO mice were dosed (bilateral ICV injection; 4 uL per hemisphere) with AAV1999-ARSA (LOT #VP091321) at a dose of 5e10 VG per mouse (1e11 VG/gm brain weight). Control age-matched WT and Arsa KO mice were dosed with formulation buffer. Mice were euthanized at 1, 2, 3 and 6 months post-dose and samples collected.

TABLE 3
Longitudinal pharmacology, efficacy and durability study in Arsa KO mice

| | Number | | | | Dosing | Time points, sample collection, |
|---|---|---|---|---|---|---|
| Group | and sex | Genotype | Test article | Dose | regimen | analysis |
| 1 | 32 (16 M, | WT | Formulation | N/A | Bi-lateral | [symbol] Dosing age: 6 mo |
| | 16 F) | | buffer | | ICV, 4 ul | [symbol] Necropsy age: 7 mo (1 mo in-life), |
| 2 | 32 (16 M, | Arsa^{-/-} | Formulation | N/A | each side | 8 mo (2 mo in-life), 9 mo (3 mo in- |
| | 16 F) | | buffer | | | life), 12 mo (6 mo in-life) |
| 3 | 32 (16 M, | Arsa^{-/-} | AAV1999- | 5e10VG | | [symbol] Brain, spinal cord, DRG, sciatic |
| | 16 F) | | ARSA (LOT# | (1e11VG/gram | | nerve, liver, CSF and plasma |
| | | | VP091321) | brain weight) | | |

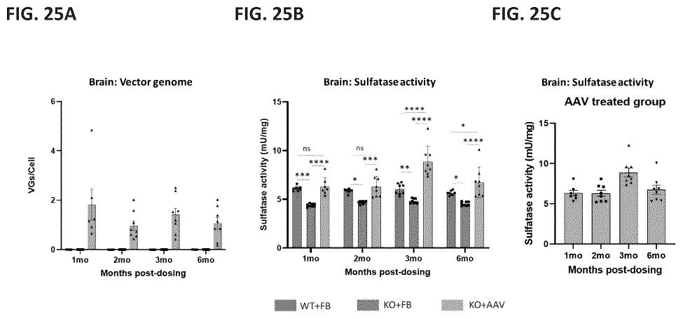

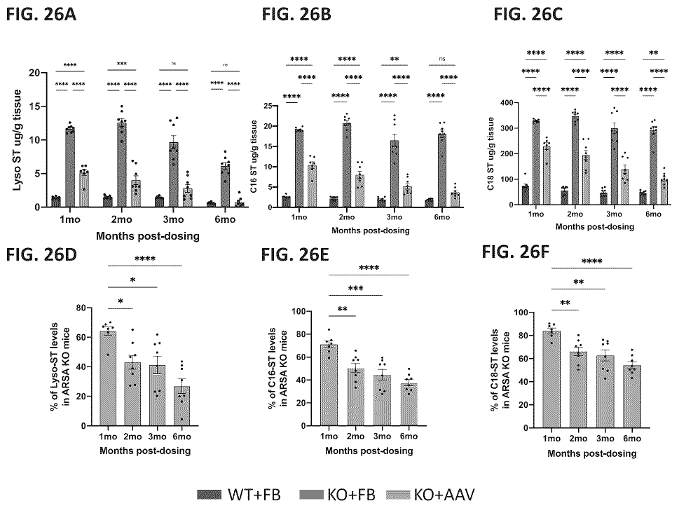

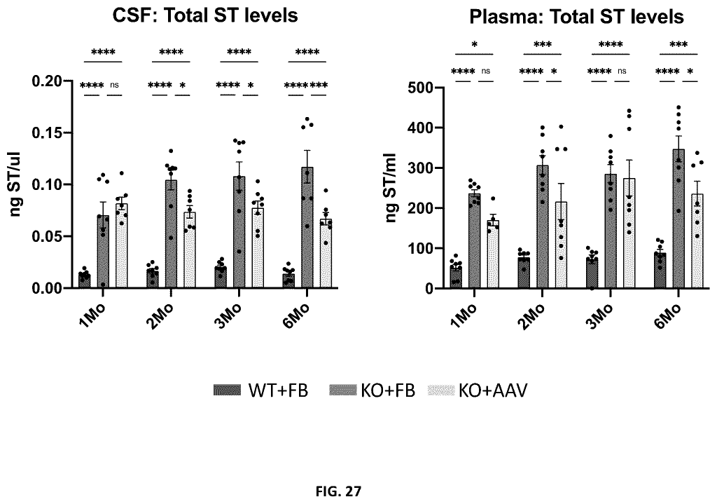

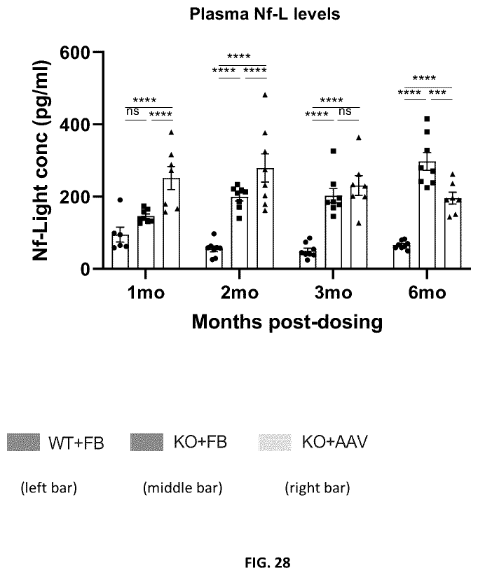

AAV1999-ARSA vector genome exposure in the brain stayed consistent over the 6-month period post-dose (FIG. 25A). This translated into persistent ARSA-mediated sulfatase activity over time (FIG. 25B) with peak sulfatase activity achieved by 1 month post-dosing (FIG. 25C) At each timepoint, treated mice showed a significant decrease in lyso-, C16- and C18-sulfatides (FIGS. 26A-C) Importantly, clearance was progressive, with sulfatide levels (especially Lyso-ST) returning to normal by 6-months post dose (FIGS. 26D-F). Furthermore, total sulfatide levels in CSF and plasma were significantly reduced in AAV1999-ARSA treated Arsa KO mice (FIG. 27). Finally, the initial increase in plasma Nf-L levels decreased by 6 months post-dosing with AAV1999-ARSA, compared to KO+FB group (FIG. 28).

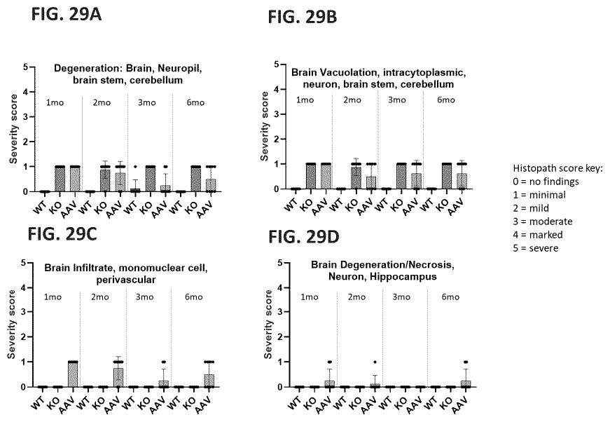

The neuronal and neutrophil changes in the brain of Arsa KO mice were observed in the brain-stem and/or cerebellar nuclei. Notably, a decrease in incidence and severity of these changes was observed in Arsa KO mice treated with AAV1999-ARSA, as early as 2 months post-dose (FIGS. 29A-B). The brain (usually near hippocampus) of some AAV1999-ARSA treated mice had focal or multifocal, perivascular, infiltrates of mononuclear cells of minimal severity (FIG. 29C). There was also minimal degeneration/necrosis of the neurons of mainly the CA3 region of the hippocampus in 1-2 animals per group (FIG. 29D).

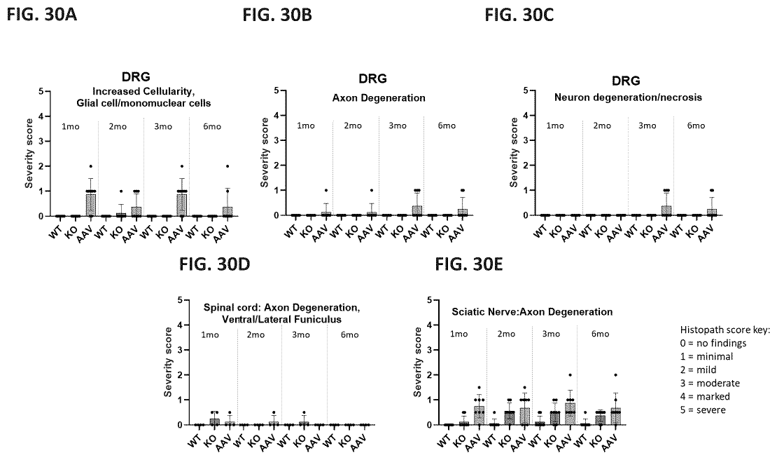

The dorsal root ganglia of some AAV1999-ARSA treated mice had focal or multifocal increase in the cellularity of glial cells and/or mononuclear cells of usually minimal severity (FIG. 30A), with some treated Arsa KO mice showing minimal degeneration of nerve axons (FIGS. 30B-C). Microscopic findings were observed in spinal cord of animals in all groups and were deemed random or incidental (FIG. 30D). Most Arsa KO mice showed histological changes (very minimal or minimal axon degeneration) in the sciatic nerve, with incidence and severity occasionally increased t*o minimal/mild or mild in AAV1999-ARSA treated Arsa KO mice (FIG. 30E). The sciatic nerve of WT mice also had rare, very minimal, background of incidental axonal degeneration (FIG. 30E). No test-article specific findings noted in the liver.

Taken together these data demonstrate that AAV1999-ARSA mediated gene replacement in Arsa KO mice is well tolerated and reversed MLD-associated phenotype to the same extent in both male and female mice.

### Example 13: NHP Pharmacology and Dose Range Finding Study Evaluating AAV1999-ARSA

Briefly, purpose bred, naive, male/female cynomolgus (Cambodia 2-3 yr old, 2.6-3.1 kg) NHPs seronegative for AAV1999 neutralizing antibodies were dosed by single direct cisterna magna (ICM) infusion. Animals were placed in Trendelenburg position during dosing. The dosing paradigm involved a single 2.5 mL infusion of AAV1999-ARSA (LOT #VP050322) at 0.125 ml/min, followed by a 250 ul flush with formulation buffer. Five weeks post-dosing, animals were euthanized, and samples were assessed for vector biodistribution (dPCR) and hARSA mRNA (RTdPCR), accompanied by a safety assessment. Samples represent 64 punches from brain representing 19 distinct grey matter regions and 7 distinct white matter regions, 8 segments of the spinal cord with adjacent DRGs, peripheral nerves and visceral organs. The study design is described in Table 4.

TABLE 4
Study CRL.2021-5613: NHP dose range finding study

| | | Dose/gm | Dose/ | Dosing | Animal | Time points, sample |
|---|---|---|---|---|---|---|
| Group | Test article | brain weight | animal | regimen | #'s | collection, analysis |
| 1 | AAV1999- | 1e10 VG | 7.5e11 VG | RoA: ICM with | 5 (M/F) | In-life: 35 days |
| | ARSA | | | animal placed | | Brain, spinal cord, |
| 2 | | 3.3e10 VG | 2.5e12 VG | in | 5 (M/F) | DRG, peripheral |
| 3 | | 1e11 VG | 7.5e12 VG | Trendelenburg | 5 (M/F) | nerves, visceral organs |
| 4 | | 3.3e11 VG | 2.5e13 VG | position | 5 (M/F) | at necropsy |
| 5 | Formulation | n/a | n/a | Doing | 3 (M/F) | CSF (pre-study, and at |
| | Buffer | | | parameters: | | necropsy) |
| | | | | 2.5 ml | | Plasma: pre-study, days |
| | | | | @0.125 mL/min | | post-dose: 2, 4, 7, 14 |
| | | | | with 250 ul | | and at necropsy |
| | | | | flush | | Neurological/behavioral |
| | | | | | | assessment (pre-dose, |
| | | | | | | 7-day and 5 wk post- |
| | | | | | | dose) |

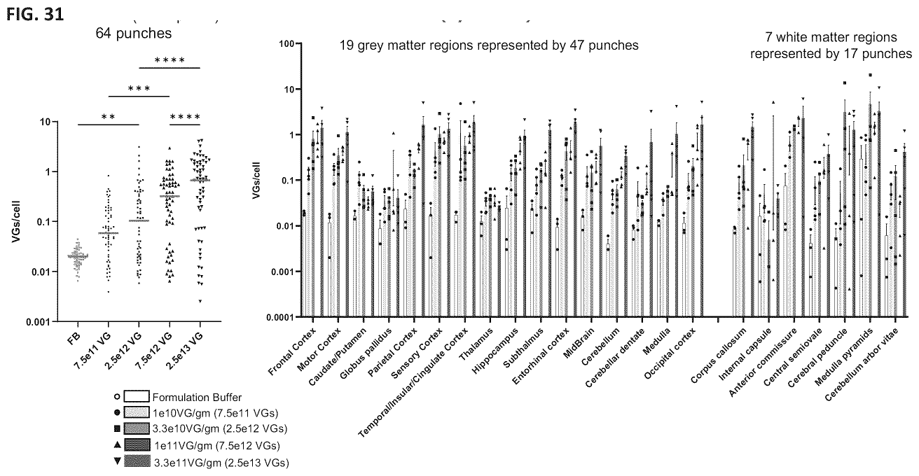

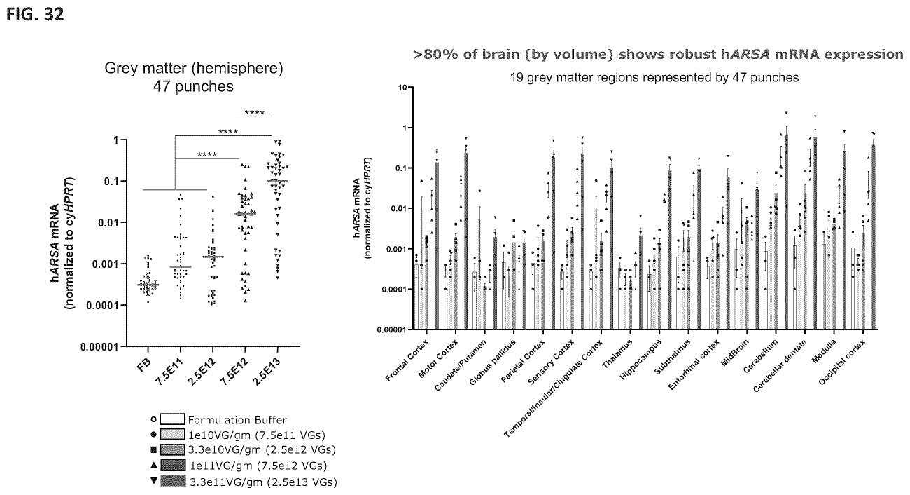

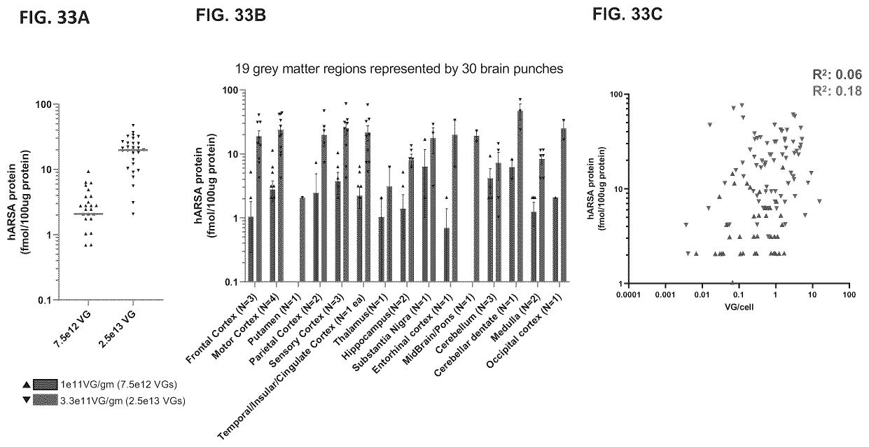

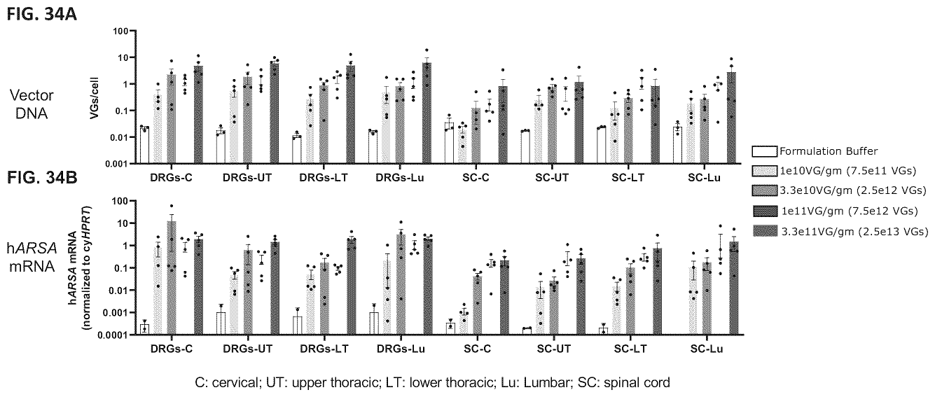

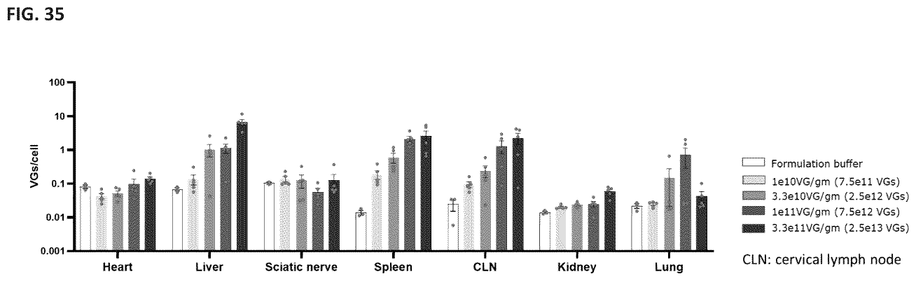

A dose-dependent increase in AAV1999-ARSA vector biodistribution was observed in NHP brain with ICM dosing, both in grey (19 unique brain regions) and white matter regions (7 unique brain regions) (FIG. 31). AAV1999-ARSA treatment also resulted in wide-spread dose-dependent increase in hARSA mRNA (FIG. 32) and protein (FIG. 33) levels at the two top doses: 7.5e12 VG (1e11 VG/gm brain weight) and 2.5e13 VG (3.3e11 VG/gm brain weight). Uniform, dose-dependent vector biodistribution and hARSA expression was observed in DRGs and spinal cord, along the spinal rostral-caudal axis (FIG. 34). Among the visceral organs, liver, spleen, and cervical lymph node show a dose-dependent increase vector biodistribution (FIG. 35).

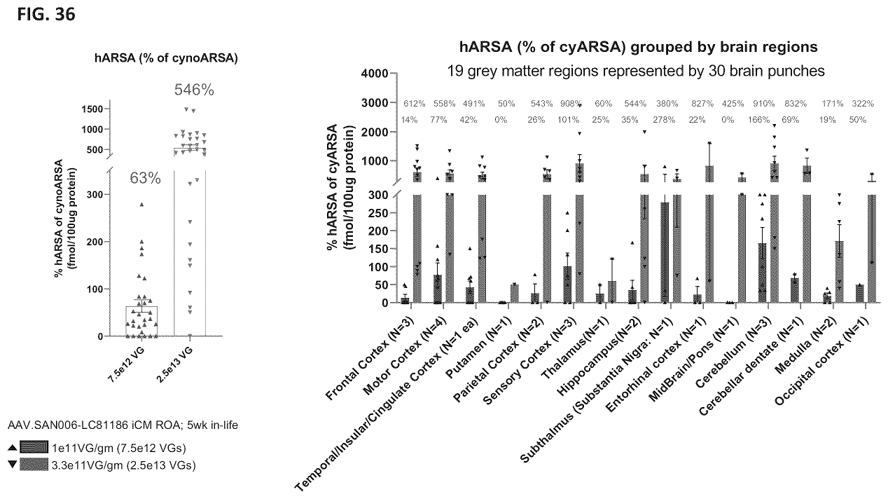

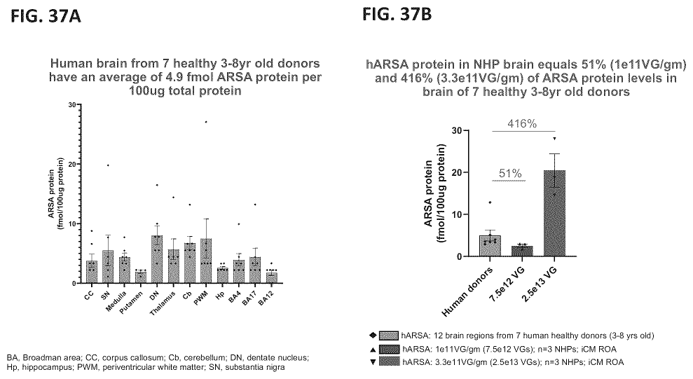

To explore whether administration of AAV1999-ARSA led to meaningful human ARSA expression in NHP brains, we assessed the level of AAV-derived human ARSA protein and compared to 1) endogenous cynomolgus cyARSA protein (hARSA/cyARSA in 19 brain regions measured within the same samples) and to 2) human ARSA protein measured in the brains (in 12 brain regions) of 7 healthy human organ-donors between the ages of 3 and 8 years old. At doses of 1e11 VG/gm and 3.3e11 VG/gm brain weight, brain-wide mean human ARSA protein levels were ˜63% and ˜546% of native cyARSA (FIG. 36). In addition, at doses of 1e11 VG/gm and 3.3e11 VG/gm brain weight, brain-wide mean human ARSA protein levels were ˜51% and ˜416% of human ARSA protein levels from healthy control organ-donors (FIG. 37). Therefore, AAV1999-ARSA treatment in NHPs results in therapeutically meaningful ARSA protein expression in the CNS at 1e11 and 3.3e11 VG/gm brain weight.

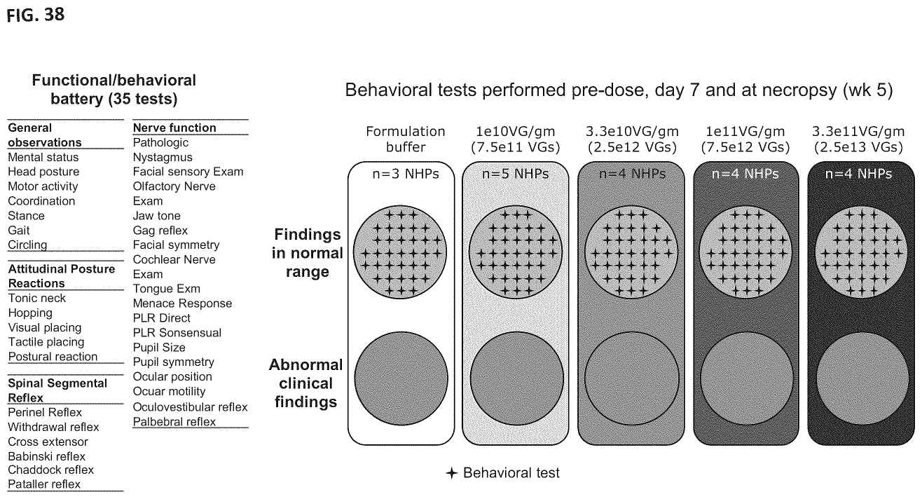

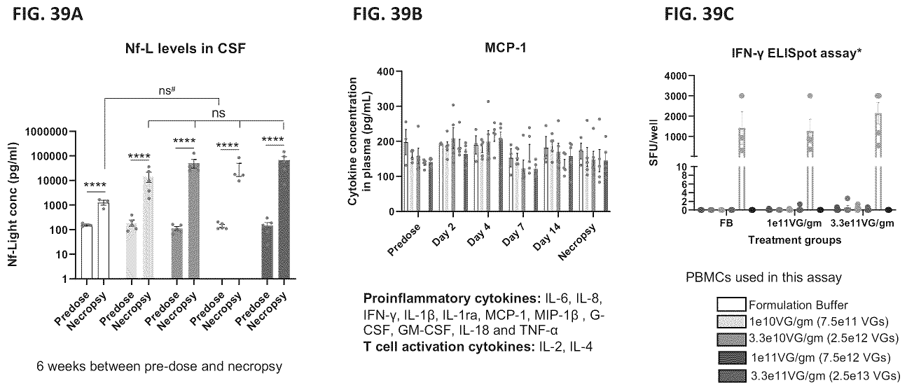

No clinical signs (functional or behavioral deficits) were observed in NHPs at week 1 or week 5 (necropsy) post-dosing in either dosing groups, compared to pre-dose tests (FIG. 38). Intra-CM infusion was well tolerated; as expected, the ICM procedure accounted for a significant increase in CSF Nf-L levels, with further increase observed in AAV1999-ARSA treated NHPs compared to formulation buffer treated NHPs (FIG. 39A). However, no dose-dependent increase was observed (FIG. 39A). No significant change in plasma cytokine concentration were observed in NHPs treated with AAV1999-ARSA across any dose (FIG. 39B), and no cell-mediated immune response was noted to AAV1999 capsid or hARSA protein in NHPs treated with AAV1999-ARSA at the two high doses by IFN-γ ELISpot (FIG. 39C). Furthermore, no test item-related gross finding noted with any animal at necropsy.

Cerebrospinal fluid changes consisted of minimal, non-dose-related, increases in nucleated cell counts (primarily of mononuclear cells) in most animals from 1e10 VG/gm brain weight. In addition, albumin and/or total proteins were increased in most animals at ≥3.3e10 VG/gm brain weight at 5-weeks post vector administration. A few individual animals at 3.3e10 or 3.3e11 VG/gm brain weight had variable amount of basophilic to eosinophilic, granular material morphologically compatible with neural material. These changes correlated with neuronal degeneration and mononuclear infiltration noted in various sections from the central nervous system.

Hematology changes consisted of transient mild increases in reticulocyte count in most males≥1e10 VG/gm brain weight and in a few sporadic females at 1e11 VG/gm brain weight on Day 7 with concurrent minimal decreases in red blood cell mass parameters in a few individuals. There were increases in white blood cell counts secondary to increases in neutrophil, lymphocyte, and/or monocyte counts in some males and females at 3.3e10 VG/gm brain weight, still noted in some animals, mainly in males administered 3.3e11 VG/gm brain weight. No test-article related effects were noted on coagulation or clinical chemistry.

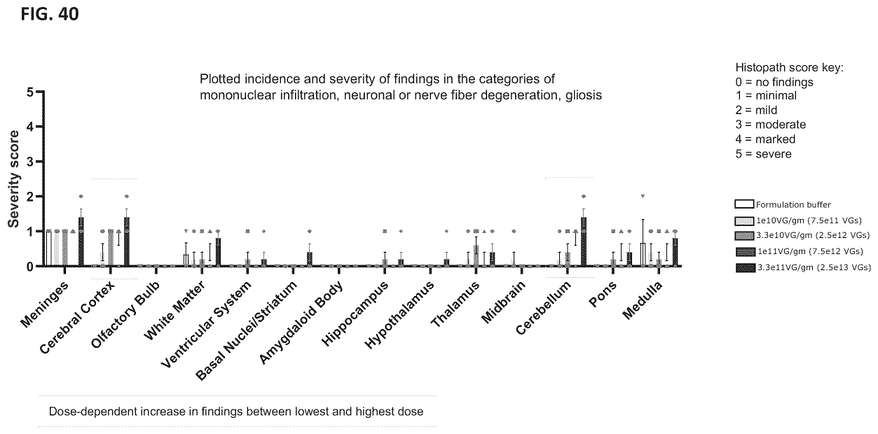

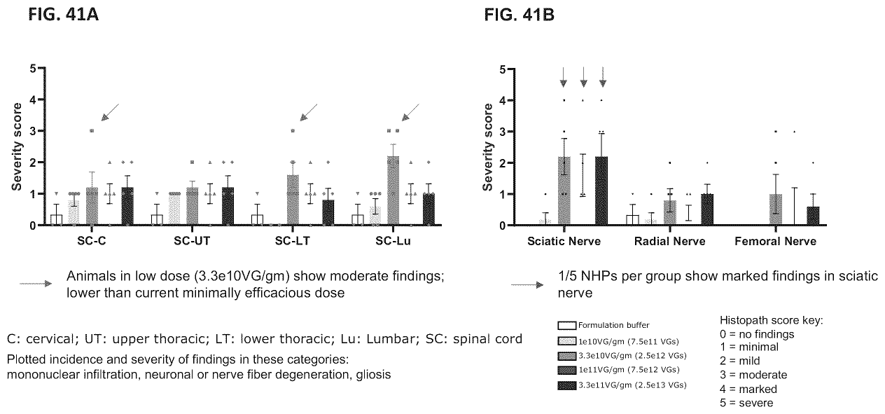

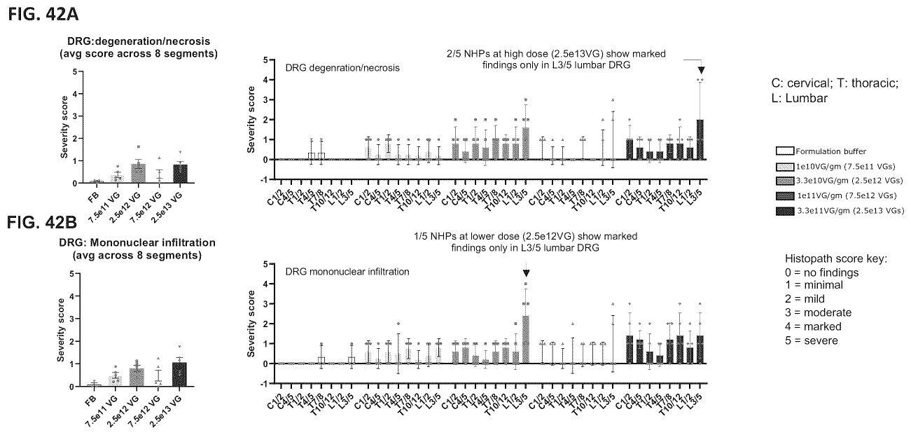

The main test article effects were comprised of neuronal degeneration (affecting brain, lumbar spinal cord, and DRG), increase glial cell response (cerebellum, and spinal cord), nerve fiber degeneration (affecting the white matter of spinal cord and nerve fibers of peripheral nerves), and mononuclear cell infiltrate (including perivascular distribution) which affected the brain (cerebellum), DRG, and spinal cord (FIGS. 40-42). No test article related findings were observed in visceral organs (Heart, liver, gallbladder, spleen, pancreas, adrenal gland, lung, bone, sternum/marrow, ovary, duodenum, testis, epididymis, thymus, eye, uterus with cervix, kidney) at any dose. A dose response was noted between the lowest dose (1e10VG/gm brain weight) and the highest dose (3.3e11 VG/gm brain weight) for some changes in some locations such as neuronal degeneration (cerebellum, lumbar DRGs at the highest dose), gliosis (cerebral cortex, cerebellum, but not in the spinal cord), and for mononuclear cell infiltrates (in the cerebral cortex and in lumbar DRGs at the highest dose) (FIGS. 40-42).

In conclusion, single administration of AAV1999-ARSA by direct intra-cisterna magna (ICM) was well tolerated in cynomolgus monkeys (both male and female) at dosing levels of 1e10, 3.3e10, 1e11 and 3.3e11 VG/gm brain weight, and 1e11 and 3.3e11 VG/gm brain weight were deemed efficacious doses.

SEQUENCES

```
ARSA Polypeptide Sequence
                10 20 30 40 50
MGAPRSLLLA LAAGLAVARP PNIVLIFADD LGYGDLGCYG HPSSTTPNLD
                60 70 80 90 100
QLAAGGLRFT DFYVPVSLCT PSRAALLTGR LPVRMGMYPG VLVPSSRGGL
              110 120 130 140 150
PLEEVTVAEV LAARGYLTGM AGKWHLGVGP EGAFLPPHQG FHRELGIPYS
              160 170 180 190 200
HDQGPCQNLT CFPPATPCDG GCDQGLVPIP LLANLSVEAQ PPWLPGLEAR
              210 220 230 240 250
YMAFAHDLMA DAQRQDRPFF LYYASHHTHY PQFSGQSFAE RSGRGPFGDS
              260 270 280 290 300
LMELDAAVGT LMTAIGDLGL LEETLVIFTA DNGPETMRMS RGGCSGLLRC
              310 320 330 340 350
GKGTTYEGGV REPALAFWPG HIAPGVTHEL ASSLDLLPTL AALAGAPLPN
              360 370 380 390 400
VTLDGFDLSP LLLGTGKSPR QSLFFYPSYP DEVRGVFAVR TGKYKAHFFT
              410 420 430 440 450
QGSAHSDTTA DPACHASSSL TAHEPPLLYD LSKDPGENYN LLGGVAGATP
              460 470 480 490 500
EVLQALKQLQ LLKAQLDAAV TFGPSQVARG EDPALQICCH PGCTPRPACC
HCPDPHA (SEQ ID NO: 1)
Codon Optimized ARSA DNA sequence (Human)
ATGAGCATGGGAGCCCCTAGATCTCTGCTGCTGGCTCTTGCTGCTGGACTGGCTGTGGCCAGACCTCCT
AACATCGTGCTGATCTTCGCCGACGATCTCGGCTATGGCGATCTGGGCTGTTACGGACACCCTAGCAGC
ACCACACCTAACCTGGATCAACTGGCTGCCGGCGGACTGAGATTCACCGATTTCTACGTGCCCGTGTCTC
TGTGCACACCTAGTAGAGCTGCTCTGCTGACAGGCAGACTGCCAGTGCGGATGGGAATGTATCCTGGCG
TGCTGGTTCCTAGCAGTAGAGGCGGACTGCCTCTGGAAGAAGTGACAGTTGCTGAAGTGCTGGCCGCC
AGAGGCTATCTGACTGGAATGGCCGGAAAATGGCACCTCGGAGTTGGACCTGAAGGCGCTTTTCTGCCT
CCTCACCAGGGCTTCCACAGATTTCTGGGCATCCCTTACAGCCACGATCAGGGCCCTTGCCAGAACCTGA
CCTGCTTTCCTCCTGCCACACCTTGTGATGGCGGCTGTGATCAGGGACTCGTGCCTATTCCTCTGCTGGCC
AATCTGAGCGTGGAAGCTCAACCTCCTTGGCTGCCTGGCCTGGAAGCCAGATATATGGCCTTCGCTCAC
GACCTGATGGCCGACGCTCAGAGACAGGACAGACCATTCTTCCTGTACTACGCCAGCCACCACACACAC
TACCCTCAGTTCTCTGGCCAGTCCTTCGCCGAGAGATCTGGCAGAGGCCCTTTTGGCGATAGCCTGATGG
AACTGGATGCCGCCGTGGGAACACTGATGACAGCCATTGGAGATCTGGGCCTGCTGGAAGAGACACTG
GTCATCTTCACCGCCGACAACGGCCCCGAGACAATGAGAATGAGCAGAGGCGGCTGTAGCGGCCTGCT
GAGATGTGGCAAGGGAACAACATACGAAGGCGGCGTCAGAGAGCCTGCTCTGGCTTTTTGGCCTGGAC
ATATTGCCCCTGGCGTGACACACGAACTGGCCTCTTCTCTGGATCTGCTGCCTACACTGGCTGCTTTGGC
TGGCGCTCCTCTGCCTAATGTGACCCTGGATGGCTTCGATCTGTCTCCACTGCTGCTCGGAACAGGCAAG
AGCCCTAGACAGAGCCTGTTCTTCTACCCTAGCTACCCCGATGAAGTGCGGGGAGTGTTTGCCGTGCGG
ACAGGCAAGTACAAGGCCCACTTTTTTACCCAAGGCAGCGCCCACAGCGATACCACAGCTGATCCTGCTT
GTCACGCCTCTAGCAGCCTGACAGCTCATGAACCACCTCTGCTGTACGACCTGTCTAAGGACCCCGGCGA
GAACTATAATCTGCTTGGCGGAGTTGCCGGCGCTACACCTGAAGTTCTGCAGGCTCTGAAACAGCTCCA
GCTGCTGAAAGCCCAGCTGGACGCTGCTGTGACATTTGGACCTTCTCAGGTGGCAAGAGGCGAGGACC
CTGCTCTGCAGATTTGTTGTCACCCTGGCTGTACCCCTAGACCTGCCTGCTGTCACTGTCCTGATCCTCAC
GCTTGA (SEQ ID NO: 2)
Full DNA sequence of plasmid for rAAV packaging; coding sequence for ARSA is
underlined
TTGGCCACTCCCTCTCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAACTCCATCACTAG
GGGTTCCTTACGTACAATTGGGATCCCGGACCGTCGACATTGATTATTGACTAGTTATTAATAGTAATCA
ATTACGGGGTCATTAGTTCATAGCCCATATATGGAGTTCCGCGTTACATAACTTACGGTAAATGGCCCGC
CTGGCTGACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGCTATTACCATGGTCGA
GGTGAGCCCCACGTTCTGCTTCACTCTCCCCATCTCCCCCCCCTCCCCACCCCCAATTTTGTATTTATTTATT
TTTTAATTATTTTGTGCAGCGATGGGGGCGGGGGGGGGGGGGGGGCGCGCGCCAGGCGGGGGGGG
CGGGGCGAGGGGCGGGGCGGGGCGAGGCGGAGAGGTGCGGCGGCAGCCAATCAGAGCGGCGCGCT
CCGAAAGTTTCCTTTTATGGCGAGGCGGCGGCGGCGGCGGCCCTATAAAAAGCGAAGCGCGCGGCGG
GCGGGAGTCGCTGCGCGCTGCCTTCGCCCCGTGCCCCGCTCCGCCGCCGCCTCGCGCCGCCCGCCCCGG
CTCTGACTGACCGCGTTACTCCCACAGGTGAGCGGGCGGGACGGCCCTTCTCCTCCGGGCTGTAATTAG
CGCTTGGTTTAATGACGGCTTGTTTCTTTTCTGTGGCTGCGTGAAAGCCTTGAGGGGCTCCGGGAGGGC
CCTTTGTGCGGGGGGAGCGGCTCGGGGGGTGCGTGCGTGTGTGTGTGCGTGGGGAGCGCCGCGTGCG
GCTCCGCGCTGCCCGGCGGCTGTGAGCGCTGCGGGCGCGGCGCGGGGCTTTGTGCGCTCCGCAGTGTG
CGCGAGGGGAGCGCGGCCGGGGGCGGTGCCCCGCGGTGCGGGGGGGGCTGCGAGGGGAACAAAGG
CTGCGTGCGGGGTGTGTGCGTGGGGGGGTGAGCAGGGGGTGTGGGCGCGTCGGTCGGGCTGCAACCC
CCCCTGCACCCCCCTCCCCGAGTTGCTGAGCACGGCCCGGCTTCGGGTGCGGGGCTCCGTACGGGGCGT
GGCGCGGGGCTCGCCGTGCCGGGCGGGGGGTGGCGGCAGGTGGGGGTGCCGGGGGGGGCGGGGCC
GCCTCGGGCCGGGGAGGGCTCGGGGGAGGGGCGCGGCGGCCCCCGGAGCGCCGGCGGCTGTCGAGG
CGCGGCGAGCCGCAGCCATTGCCTTTTATGGTAATCGTGCGAGAGGGCGCAGGGACTTCCTTTGTCCCA
AATCTGTGCGGAGCCGAAATCTGGGAGGCGCCGCCGCACCCCCTCTAGCGGGCGCGGGGCGAAGCGG
TGCGGCGCCGGCAGGAAGGAAATGGGGGGGGAGGGCCTTCGTGCGTCGCCGCGCCGCCGTCCCCTTCT
CCCTCTCCAGCCTCGGGGCTGTCCGCGGGGGGACGGCTGCCTTCGGGGGGGACGGGGCAGGGCGGGG
TTCGGCTTCTGGCGTGTGACCGGCGGCTCTAGAGCCTCTGCTAACCATGTTCATGCCTTCTTCTTTTTCCT
ACAGCTCCTGGGCAACGTGCTGGTTATTGTGCTGTCTCATCATTTTGGCAAAGAATTCTACGTACCACCA
TGAGCATGGGAGCCCCTAGATCTCTGCTGCTGGCTCTTGCTGCTGGACTGGCTGTGGCCAGACCTCCTA
ACATCGTGCTGATCTTCGCCGACGATCTCGGCTATGGCGATCTGGGCTGTTACGGACACCCTAGCAGCA
CCACACCTAACCTGGATCAACTGGCTGCCGGCGGACTGAGATTCACCGATTTCTACGTGCCCGTGTCTCT
GTGCACACCTAGTAGAGCTGCTCTGCTGACAGGCAGACTGCCAGTGCGGATGGGAATGTATCCTGGCGT
GCTGGTTCCTAGCAGTAGAGGCGGACTGCCTCTGGAAGAAGTGACAGTTGCTGAAGTGCTGGCCGCCA
GAGGCTATCTGACTGGAATGGCCGGAAAATGGCACCTCGGAGTTGGACCTGAAGGCGCTTTTCTGCCTC
CTCACCAGGGCTTCCACAGATTTCTGGGCATCCCTTACAGCCACGATCAGGGCCCTTGCCAGAACCTGAC
CTGCTTTCCTCCTGCCACACCTTGTGATGGCGGCTGTGATCAGGGACTCGTGCCTATTCCTCTGCTGGCC
AATCTGAGCGTGGAAGCTCAACCTCCTTGGCTGCCTGGCCTGGAAGCCAGATATATGGCCTTCGCTCAC
GACCTGATGGCCGACGCTCAGAGACAGGACAGACCATTCTTCCTGTACTACGCCAGCCACCACACACAC
TACCCTCAGTTCTCTGGCCAGTCCTTCGCCGAGAGATCTGGCAGAGGCCCTTTTGGCGATAGCCTGATGG
AACTGGATGCCGCCGTGGGAACACTGATGACAGCCATTGGAGATCTGGGCCTGCTGGAAGAGACACTG
GTCATCTTCACCGCCGACAACGGCCCCGAGACAATGAGAATGAGCAGAGGCGGCTGTAGCGGCCTGCT
GAGATGTGGCAAGGGAACAACATACGAAGGCGGCGTCAGAGAGCCTGCTCTGGCTTTTTGGCCTGGAC
ATATTGCCCCTGGCGTGACACACGAACTGGCCTCTTCTCTGGATCTGCTGCCTACACTGGCTGCTTTGGC
TGGCGCTCCTCTGCCTAATGTGACCCTGGATGGCTTCGATCTGTCTCCACTGCTGCTCGGAACAGGCAAG
AGCCCTAGACAGAGCCTGTTCTTCTACCCTAGCTACCCCGATGAAGTGCGGGGAGTGTTTGCCGTGCGG
ACAGGCAAGTACAAGGCCCACTTTTTTACCCAAGGCAGCGCCCACAGCGATACCACAGCTGATCCTGCTT
GTCACGCCTCTAGCAGCCTGACAGCTCATGAACCACCTCTGCTGTACGACCTGTCTAAGGACCCCGGCGA
GAACTATAATCTGCTTGGCGGAGTTGCCGGCGCTACACCTGAAGTTCTGCAGGCTCTGAAACAGCTCCA
GCTGCTGAAAGCCCAGCTGGACGCTGCTGTGACATTTGGACCTTCTCAGGTGGCAAGAGGCGAGGACC
CTGCTCTGCAGATTTGTTGTCACCCTGGCTGTACCCCTAGACCTGCCTGCTGTCACTGTCCTGATCCTCAC
GCTTGAGATTAATCAACCTCTGGATTACAAAATTTGTGAAAGATTGACTGGTATTCTTAACTATGTTGCTC
CTTTTACGCTATGTGGATACGCTGCTTTAATGCCTTTGTATCATGCTATTGCTTCCCGTATGGCTTTCATTT
TCTCCTCCTTGTATAAATCCTGGTTGCTGTCTCTTTATGAGGAGTTGTGGCCCGTTGTCAGGCAACGTGG
CGTGGTGTGCACTGTGTTTGCTGACGCAACCCCCACTGGTTGGGGCATTGCCACCACCTGTCAGCTCCTT
TCCGGGACTTTCGCTTTCCCCCTCCCTATTGCCACGGCGGAACTCATCGCCGCCTGCCTTGCCCGCTGCTG
GACAGGGGCTCGGCTGTTGGGCACTGACAATTCCGTGGTGTTGTCGGGGAAATCATCGTCCTTTCCTTG
GCTGCTCGCCTGTGTTGCCACCTGGATTCTGCGCGGGACGTCCTTCTGCTACGTCCCTTCGGCCCTCAATC
CAGCGGACCTTCCTTCCCGCGGCCTGCTGCCGGCTCTGCGGCCTCTTCCGCGTCTTCGCCTTCGCCCTCAG
ACGAGTCGGATCTCCCTTTGGGCCGCCTCCCCGCCTGATCCTGTGCCTTCTAGTTGCCAGCCATCTGTTGT
TTGCCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAG
GGGGAGGATTGGGAAGACAATAGCAGGCATGCTCGAAATTAGGAACCCCTAGTGATGGAGTTGGCCAC
TCCCTCTCTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAATCGCGACGGCCGACGTCTTT
GTTACAACTTACTATATATATGCACACATATATATATATTTGGGTATATTGGGGGGGTTCTAATTTAAGAA
ATGCATAATTGGCTATAGACAGACAGTTGTCAGAACTTGGCAATGGGTACGTGCAGGTTCATTATACCA
AGTCTACTTGTAGTTGTTCAAAATGTATCATAATACAAGGCCGGGCGAGGTCGTCACGCCTGTAATCCCA
GCATTTTGGGAGGCTAAGGCAGGAGGATTGCTTGAGGTCAGGAGTTTGTGACCAGCCTGGGCAACAGA
GCAAGACCCTGTCTCCAAAAAGAAAAAAAATAATTTTTTACAAAATAAAAACAAAATGTATCATCAGACG
AAATTAAATAAGAGGCAATTCATTGTAATGACAACTTTTCCCAGCTTGACATTTAACAAAAAGTCTAAGT
CCTCTTAATTCATATTTAATGATCAAATATCAAATACTAATTTTTTTTTTTTTTTTTTTTTTGAGACGGAGTC
TCGCTCTGTCGCCCAGGCTGGAGTGCAGTGGCGCGATCCTGGCTCACTGCAAGCTCCGCCTCCCGGGTT
CACGCCATTCTCCTGCCTCAGCCTCCCGAGTAGCTGGGATTACAGACATGCGCCACCACGCCCGGCTAAT
TTTGTATTTTTAGTAGAGATGGGGTTTCTCCATGTTGGTCAGGCTGGTCTTGAATTTCCCACCTCAGGTGA
TCTGCCTGCCTCAGCCTCACAAAGCAGTAGCTGGGACTACAGGCACCCACCACCACACTTGGTTAATTCT
TTTGTATTTTTTTTGTAAAGACGGGATTTCACCATGTTAGCCAGGATGGTCTCGATCTCCTGATCTCATGA
TCCGCCCGCCTCAGCCTCCCAAAGTGCTGGGATTACAGGCGTGAGCCACCCCGCCCGGCCATCAAATAC
TAATTCTTAAATGGTAAGGACCCACTATTCAGAACCTGTATCCTTATCACTAATATGCAAATATTTATTGA
ATACTTACTATGTCATGCATACTAGAGAGAGTTAGATAAATTTGATACAGCTACCCTCACAGAACTTACA
GTGTAATAGATGGCATGACATGTACATGAGTAACTGTGAACAGTGTTAAATTGCTATTTAAAAAAAAAG
ACGGCTGGGCGCTGTGGCTCATGCCTGTAATCCCAGCACTTTGGGAGGCCAAGGCAAGTTGATCGCTCG
AGGTCAAGAGTTCGAGACCAGCCTGGCCAACGTGGTAAAACCCCGTCTCTACTAAAAATACAAAAAAAA
AATTAGCCAGGCATGGTGGCACAGGCCTGTAATCCCAGCTACTAGGGAGGCTGAGACATGGAGAACTG
CTTGAATCCAGGAGGCAGAGGTTACAGTGAGCCGAGATCATACCACTACACTCCAGCCTGAGTGACAGA
GCGAGACTCCTGTCTAAAAAAAAAAAAAAAAAAAAAGATACAGGTTAAGTGTTATGGTAGTTGAAGAG
AGAACTCAAACTCTGTCTCAGAAGCCTCACTTGCATGTGGACCACTGATATGAAATAATATAAATAGGTA
TAATTCAATAAATAGGAACTTCAGTTTTAATCATCCCAAACACCAAAACTTCCTATCAAACAGGTCCAATA
AACTCAATCTCTATAAGAGCTAGACAGAAATCTACTTGGTGGCCTATAATCTTATTAGCCCTTACTTGTCC
CATCTGATATTAATTAACCCCATCTAATATGGATTAGTTAACAATCCAGTGGCTGCTTTGACAGGAACAG
TTGGAGAGAGTTGGGGATTGCAACATATTCAATTATACAAAAATGCATTCAGCATCTACCTTGATTAAGG
CAGTGTGCAACAGAATTTGCAGGAGAGTAAAAGAATGATTATAAATTTACAACCCTTAAAGAGCTATAG
CTGGGCGTGGTGGCTCATGCCTGTAAATCCCAGCACTTTGGGAGGCTGAGGCGGGTGGATCACCTGAG
GCCAGAAGTTCAAGACCAGCCTAGCCAACATGGCGAAACCCTGTCTCTACAAAAAATACAAAAATTAGC
CGGGTGTGGTGGCACGTGCCTGTAGTCCCAGTTACTTGGGAGGCCGAGGCAGGAGAATCGCTTGAACC
TAGGAGGTGGAGGCTGCAGTGAGCCGAGATTGTGCCACTGCACTCCACTTCAGCCTGGGCGACAAGAG
CAAGACTCCGTCACAAAAAAAAAAAAAAAAAAAAAGGCTAAAATCTAGTGGGAAAGGCATATATACAT
ACAACTAACTGTATAGCATAATAAAGCTCATAATCTGTAACAAAATCTAATTCGACAAGCCCAGAAACTT
GTGATTTACCAAAAACAGTTATATATACACAAAAAGTAAACCTAGAACCCAAAGTTACCCAGCACCAATG
ATTCTCTCCCTAAGCAGTATCAAGTTTAAAGCAGTGATTACATTCTACTGCCTAGATTGTAAACTGAGTAA
AGGAGACCAGCACCTTTCTGCTACTGAACTAGCACAGCCGTGTAAACCAACAAGGCAATGGCAGTGCCC
AACTTTCTGTATGAATATAAGTTACATCTGTTTTATTATTTGTGACTTGGTGTTGCATGTGGTTATTATCAA
CACCTTCTGAAAGAACAACTACCTGCTCAGGCTGCCATAACAAAATACCACAGACTGAGTGACTTAACAG
AAACTTATTTCTCACAGTTTTGGAGGCTGGGAAGTCCAAAATTAAGCTAACTGCAAGGTAGGTTTCAATC
TCAGGCCTCTTCTTTGGCTTGAAGGTCTTCTAACTGTGTGCTCACATGACCTCTTCTAACAAGCTCTCTGG
TGTCTCTTTTTTTTTTTTTTTCTTTTTTGAGACAGAGTCTCACTCTGTCACCCAGGCTGGAGTACAGTGGCA
CAATCTGGGCTCACTGCAACCTCCAACTCCCGGGTTCAAGTGATTCTCATGCCTCACCCTCCCGAGTAGCT
TGGATGACAGGAGCCCGCTACCACACCCAGCTAATTTTTGTATTTTTAGTAGAGATGGTGTTTCACTACA
TTGGCCAGGCTGGTCTCAAACTCCTGACCTCGTGATCCACCCACCTTGGCCTCCCAAAGTGCTGGGATTA
CAGGTGTGAGCCACTGCGCCCGTCCTGGTGTCTTTTCATATAAGGGCACTAATCCAATCAGACCTGGGCC
CAACCCTCCCGACTTCTTCTAACTGTAATTACCTTCCAAAGGCCCTGTCTCCAAATACCATCACACTGGGG
GTTAGGACTTCAAAAAAGGTATGGGGGGGGTGTGGGAGGACATAAATGCTCAGTCCATAACAAGCACC
CAACATAAAAATGGCTAGAACAGATCACAAAAAAAAGGTCCTGTATGGCTTTGGGGAAGGGCTCAACC
CCAAAATATCTGAAAGCTCTGGAGGGGCCTAGAAGTGGTAAATGAATGAAAACGTGGTTACTCTCCCGA
TCTGCCTTTCCCAAATATGGCCATTCTTGGCTGAATCAGAAATCAAAGGACAGGTTATTAATTACTAGCTC
TAAGTTACTTACCATTTGCTGAGACAGTTCAGAAATCTGACTGCATCTCCTCAGGGATCTAGAACACAGT
TCTCAAATTCTAACTTACTTGTGATATACTTGTGAATGATAAAAATCGCTACAGGTACTTTTATTAATCTG
AAAGAGTATTGAGAAATTACCTTTCATTCTGACTTTTGTCTGGAATGAAAATCAATACTTTTGCTATATTC
CATTACTGAAATAATTTTACTTTCCAGTAAAACTGGCATTATAATTTTTTTTAATTTTTAAAACTTCATAATT
TTTTGCCAGACTGACCCATGTAAACATACAAATTACTAATAATTATGCACGTCACATCTGTAATAATGGCC
TTCATGTAAACATTTTTGTGGTTTACACATAAAATCTCTAATTACAAAGCTATATTATCTAAAATTACAGTA
AGCAAGAAAATTAATCCAAGCTAAGACAATACTTGCAACATCAATTCATCATCTGTGACAAGGACTGCTT
AAGTCTCTTTGTGGTTGACGTCATTAATTAACTGGCCTCATGGGCCTTCCGCTCACTGCCCGCTTTCCAGT
CGGGAAACCTGTCGTGCCAGCTGCATTAACATGGTCATAGCTGTTTCCTTGCGTATTGGGCGCTCTCCGC
TTCCTCGCTCACTGACTCGCTGCGCTCGGTCGTTCGGGTAAAGCCTGGGGTGCCTAATGAGCAAAAGGC
CAGCAAAAGGCCAGGAACCGTAAAAAGGCCGCGTTGCTGGCGTTTTTCCATAGGCTCCGCCCCCCTGAC
GAGCATCACAAAAATCGACGCTCAAGTCAGAGGTGGCGAAACCCGACAGGACTATAAAGATACCAGGC
GTTTCCCCCTGGAAGCTCCCTCGTGCGCTCTCCTGTTCCGACCCTGCCGCTTACCGGATACCTGTCCGCCT
TTCTCCCTTCGGGAAGCGTGGCGCTTTCTCATAGCTCACGCTGTAGGTATCTCAGTTCGGTGTAGGTCGT
TCGCTCCAAGCTGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAACAGGATTAGC
AGAGCGAGGTATGTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCGCAGAAAAAAA
GGATCTCAAGAAGATCCTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATTAAAAATGAAGTTTTAA
ATCAATCTAAAGTATATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATAACTACGATACGGGA
GGGCTTACCATCTGGCCCCAGTGCTGCAATGATACCGCGAGAACCACGCTCACCGGCTCCAGATTTATCA
GCAATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTTGCCA
TTGCTACAGGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGATCGTTGTC
AGAAGTAAGTTGGCCGCAGTGTTATCACTCATGGTTATGGCAGCACTGCATAATTCTCTTACTGTCATGC
CATCCGTAAGATGCTTTTCTGTGACTGGTGAGTACTCAACCAAGTCATTCTGAGAATAGTGTATGCGGCG
ACCGAGTTGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGAATACTCATACTCTTCC
TTTTTCAATATTATTGAAGCATTTATCAGGGTTATTGTCTCATGAGCGGATACATATTTGAATGTATTTAG
AAAAATAAACAAATAGGGGTTCCGCGCACATTTCCCCGAAAAGTGCCACCTAAATTGTAAGCGTTAATAT
TTTGTTAAAATTCGCGTTAAATTTTTGTTAAATCAGCTCATTTTTTAACCAATAGGCCGAAATCGGCAAAA
TCCCTTATAAATCAAAAGAATAGACCGAGATAGGGTTGAGTGGCCGCTACAGGGCGCTCCCATTCGCCA
TTCAGGCTGCGCAACTGTTGGGAAGGGCGTTTCGGTGCGGGCCTCTTCGCTATTACGCCAGCTGGCGAA
AGGGGGATGTGCTGCAAGGCGATTAAGTTGGGTAACGCCAGGGTTTTCCCAGTCACGACGTTGTAAAA
CGACGGCCAGTGAGCGCGACGTAATACGACTCACTATAGGGCGAATTGGCGGAAGGCCGTCAAGGCCT
AGGCGCGCCAACGCGT (SEQ ID NO: 3)
Underlined, codon Optimized ARSA DNA sequence (Human)
5′ AAV2 ITR DNA Sequence
TTGGCCACTCCCTCTCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAACTCCATCACTAG
GGGTTCCT (SEQ ID NO: 4)
3′ AAV2 ITR DNA Sequence in Flip Orientation
AGGAACCCCTAGTGATGGAGTTGGCCACTCCCTCTCTGCGCGCTCGCTCGCTCACTGAGGCCGCCCGGG
CAAAGCCCGGGCGTCGGGCGACCTTTGGTCGCCCGGCCTCAGTGAGCGAGCGAGCGCGCAGAGAGGG
AGTGGCCAA (SEQ ID NO: 5)
CMV Enhancer Element DNA Sequence (GenBank: K03104.1)
GACATTGATTATTGACTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGGAGTATT
TACGGTAAACTGCCCACTTGGCAGTACATCAAGTGTATCATATGCCAAGTACGCCCCCTATTGACGTCAA
TGACGGTAAATGGCCCGCCTGGCATTATGCCCAGTACATGACCTTATGGGACTTTCCTACTTGGCAGTAC
ATCTACGTATTAGTCATCGCTATTACCATGG (SEQ ID NO: 6)
Chicken-Actin Promoter DNA Sequence
TCGAGGTGAGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGGGCGCGCGCCAGGCGGGGC
GGGGCGGGGCGAGGGGCGGGGCGGGGCGAGGCGGAGAGGTGCGGCGGCAGCCAATCAGAGCGGCG
CGCTCCGAAAGTTTCCTTTTATGGCGAGGCGGCGGCGGCGGCGGCCCTATAAAAAGCGAAGCGCGCGG
CGGGCGGGAGTCGCTGCGCGCTGCCTTCGCCCCGTGCCCCGCTCCGCCGCC (SEQ ID NO: 7)
WPRE Element DNA Sequence
TTAATCAACCTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCATTTTCTCCTCC
TTGTATAAATCCTGGTTGCTGTCTCTTTATGAGGAGTTGTGGCCCGTTGTCAGGCAACGTGGCGTGGTGT
GCACTGTGTTTGCTGACGCAACCCCCACTGGTTGGGGCATTGCCACCACCTGTCAGCTCCTTTCCGGGAC
TTTCGCTTTCCCCCTCCCTATTGCCACGGCGGAACTCATCGCCGCCTGCCTTGCCCGCTGCTGGACAGGG
GCTCGGCTGTTGGGCACTGACAATTCCGTGGTGTTGTCGGGGAAATCATCGTCCTTTCCTTGGCTGCTCG
CCTGTGTTGCCACCTGGATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTTCGCCCTCAGACGAGTC
GGATCTCCCTTTGGGCCGCCTCCCCGCCTG (SEQ ID NO: 8)
AAV9 Capsid Amino Acid Sequence (VP1)
Met Ala Ala Asp Gly Tyr Leu Pro Asp Trp Leu Glu Asp Asn Leu Ser
1 5 10 15
Glu Gly Ile Arg Glu Trp Trp Ala Leu Lys Pro Gly Ala Pro Gln Pro
                        20 25 30
Lys Ala Asn Gln Gln His Gln Asp Asn Ala Arg Gly Leu Val Leu Pro
                35 40 45
Gly Tyr Lys Tyr Leu Gly Pro Gly Asn Gly Leu Asp Lys Gly Glu Pro
        50 55 60
Val Asn Ala Ala Asp Ala Ala Ala Leu Glu His Asp Lys Ala Tyr Asp
65 70 75 80
Gln Gln Leu Lys Ala Gly Asp Asn Pro Tyr Leu Lys Tyr Asn His Ala
                                85 90 95
Asp Ala Glu Phe Gln Glu Arg Leu Lys Glu Asp Thr Ser Phe Gly Gly
                        100 105 110
Asn Leu Gly Arg Ala Val Phe Gln Ala Lys Lys Arg Leu Leu Glu Pro
                115 120 125
Leu Gly Leu Val Glu Glu Ala Ala Lys Thr Ala Pro Gly Lys Lys Arg
        130 135 140
Pro Val Glu Gln Ser Pro Gln Glu Pro Asp Ser Ser Ala Gly Ile Gly
145 150 155 160
Lys Ser Gly Ala Gln Pro Ala Lys Lys Arg Leu Asn Phe Gly Gln Thr
                                165 170 175
Gly Asp Thr Glu Ser Val Pro Asp Pro Gln Pro Ile Gly Glu Pro Pro
                        180 185 190
Ala Ala Pro Ser Gly Val Gly Ser Leu Thr Met Ala Ser Gly Gly Gly
                195 200 205
Ala Pro Val Ala Asp Asn Asn Glu Gly Ala Asp Gly Val Gly Ser Ser
        210 215 220
Ser Gly Asn Trp His Cys Asp Ser Gln Trp Leu Gly Asp Arg Val Ile
225 230 235 240
Thr Thr Ser Thr Arg Thr Trp Ala Leu Pro Thr Tyr Asn Asn His Leu
                                245 250 255
Tyr Lys Gln Ile Ser Asn Ser Thr Ser Gly Gly Ser Ser Asn Asp Asn
                        260 265 270
Ala Tyr Phe Gly Tyr Ser Thr Pro Trp Gly Tyr Phe Asp Phe Asn Arg
                275 280 285
Phe His Cys His Phe Ser Pro Arg Asp Trp Gln Arg Leu Ile Asn Asn
        290 295 300
Asn Trp Gly Phe Arg Pro Lys Arg Leu Asn Phe Lys Leu Phe Asn Ile
305 310 315 320
Gln Val Lys Glu Val Thr Asp Asn Asn Gly Val Lys Thr Ile Ala Asn
                                325 330 335
Asn Leu Thr Ser Thr Val Gln Val Phe Thr Asp Ser Asp Tyr Gln Leu
                        340 345 350
Pro Tyr Val Leu Gly Ser Ala His Glu Gly Cys Leu Pro Pro Phe Pro
                355 360 365
Ala Asp Val Phe Met Ile Pro Gln Tyr Gly Tyr Leu Thr Leu Asn Asp
        370 375 380
Gly Ser Gln Ala Val Gly Arg Ser Ser Phe Tyr Cys Leu Glu Tyr Phe
385 390 395 400
Pro Ser Gln Met Leu Arg Thr Gly Asn Asn Phe Gln Phe Ser Tyr Glu
                                405 410 415
Phe Glu Asn Val Pro Phe His Ser Ser Tyr Ala His Ser Gln Ser Leu
                        420 425 430
Asp Arg Leu Met Asn Pro Leu Ile Asp Gln Tyr Leu Tyr Tyr Leu Ser
                435 440 445
Lys Thr Ile Asn Gly Ser Gly Gln Asn Gln Gln Thr Leu Lys Phe Ser
        450 455 460
Val Ala Gly Pro Ser Asn Met Ala Val Gln Gly Arg Asn Tyr Ile Pro
465 470 475 480
Gly Pro Ser Tyr Arg Gln Gln Arg Val Ser Thr Thr Val Thr Gln Asn
                                485 490 495
Asn Asn Ser Glu Phe Ala Trp Pro Gly Ala Ser Ser Trp Ala Leu Asn
                        500 505 510
Gly Arg Asn Ser Leu Met Asn Pro Gly Pro Ala Met Ala Ser His Lys
                515 520 525
Glu Gly Glu Asp Arg Phe Phe Pro Leu Ser Gly Ser Leu Ile Phe Gly
        530 535 540
Lys Gln Gly Thr Gly Arg Asp Asn Val Asp Ala Asp Lys Val Met Ile
545 550 555 560
Thr Asn Glu Glu Glu Ile Lys Thr Thr Asn Pro Val Ala Thr Glu Ser
                                565 570 575
Tyr Gly Gln Val Ala Thr Asn His Gln Ser Ala Gln Ala Gln Ala Gln
                        580 585 590
Thr Gly Trp Val Gln Asn Gln Gly Ile Leu Pro Gly Met Val Trp Gln
                595 600 605
Asp Arg Asp Val Tyr Leu Gln Gly Pro Ile Trp Ala Lys Ile Pro His
        610 615 620
Thr Asp Gly Asn Phe His Pro Ser Pro Leu Met Gly Gly Phe Gly Met
625 630 635 640
Lys His Pro Pro Pro Gln Ile Leu Ile Lys Asn Thr Pro Val Pro Ala
                                645 650 655
Asp Pro Pro Thr Ala Phe Asn Lys Asp Lys Leu Asn Ser Phe Ile Thr
                        660 665 670
Gln Tyr Ser Thr Gly Gln Val Ser Val Glu Ile Glu Trp Glu Leu Gln
                675 680 685
Lys Glu Asn Ser Lys Arg Trp Asn Pro Glu Ile Gln Tyr Thr Ser Asn
        690 695 700
Tyr Tyr Lys Ser Asn Asn Val Glu Phe Ala Val Asn Thr Glu Gly Val
705 710 715 720
Tyr Ser Glu Pro Arg Pro Ile Gly Thr Arg Tyr Leu Thr Arg Asn Leu
                                725 730 735
(SEQ ID NO: 9)
Targeting Peptide Amino Acid Sequence
KGGGFHG (SEQ ID NO: 10)
Targeting Peptide Flanked by Linkers-Amino Acid Sequence
AAAKGGGFHGAS (SEQ ID NO: 11)
AAV1999 Capsid Amino Acid Sequence (VP1)
Met Ala Ala Asp Gly Tyr Leu Pro Asp Trp Leu Glu Asp Asn Leu Ser
1 5 10 15
Glu Gly Ile Arg Glu Trp Trp Ala Leu Lys Pro Gly Ala Pro Gln Pro
                        20 25 30
Lys Ala Asn Gln Gln His Gln Asp Asn Ala Arg Gly Leu Val Leu Pro
                35 40 45
Gly Tyr Lys Tyr Leu Gly Pro Gly Asn Gly Leu Asp Lys Gly Glu Pro
        50 55 60
Val Asn Ala Ala Asp Ala Ala Ala Leu Glu His Asp Lys Ala Tyr Asp
65 70 75 80
Gln Gln Leu Lys Ala Gly Asp Asn Pro Tyr Leu Lys Tyr Asn His Ala
                                85 90 95
Asp Ala Glu Phe Gln Glu Arg Leu Lys Glu Asp Thr Ser Phe Gly Gly
                        100 105 110
Asn Leu Gly Arg Ala Val Phe Gln Ala Lys Lys Arg Leu Leu Glu Pro
                115 120 125
Leu Gly Leu Val Glu Glu Ala Ala Lys Thr Ala Pro Gly Lys Lys Arg
        130 135 140
Pro Val Glu Gln Ser Pro Gln Glu Pro Asp Ser Ser Ala Gly Ile Gly
145 150 155 160
Lys Ser Gly Ala Gln Pro Ala Lys Lys Arg Leu Asn Phe Gly Gln Thr
                                165 170 175
Gly Asp Thr Glu Ser Val Pro Asp Pro Gln Pro Ile Gly Glu Pro Pro
                        180 185 190
Ala Ala Pro Ser Gly Val Gly Ser Leu Thr Met Ala Ser Gly Gly Gly
                195 200 205
Ala Pro Val Ala Asp Asn Asn Glu Gly Ala Asp Gly Val Gly Ser Ser
        210 215 220
Ser Gly Asn Trp His Cys Asp Ser Gln Trp Leu Gly Asp Arg Val Ile
225 230 235 240
Thr Thr Ser Thr Arg Thr Trp Ala Leu Pro Thr Tyr Asn Asn His Leu
                                245 250 255
Tyr Lys Gln Ile Ser Asn Ser Thr Ser Gly Gly Ser Ser Asn Asp Asn
                        260 265 270
Ala Tyr Phe Gly Tyr Ser Thr Pro Trp Gly Tyr Phe Asp Phe Asn Arg
                275 280 285
Phe His Cys His Phe Ser Pro Arg Asp Trp Gln Arg Leu Ile Asn Asn
        290 295 300
Asn Trp Gly Phe Arg Pro Lys Arg Leu Asn Phe Lys Leu Phe Asn Ile
305 310 315 320
Gln Val Lys Glu Val Thr Asp Asn Asn Gly Val Lys Thr Ile Ala Asn
                                325 330 335
Asn Leu Thr Ser Thr Val Gln Val Phe Thr Asp Ser Asp Tyr Gln Leu
                        340 345 350
Pro Tyr Val Leu Gly Ser Ala His Glu Gly Cys Leu Pro Pro Phe Pro
                355 360 365
Ala Asp Val Phe Met Ile Pro Gln Tyr Gly Tyr Leu Thr Leu Asn Asp
        370 375 380
Gly Ser Gln Ala Val Gly Arg Ser Ser Phe Tyr Cys Leu Glu Tyr Phe
385 390 395 400
Pro Ser Gln Met Leu Arg Thr Gly Asn Asn Phe Gln Phe Ser Tyr Glu
                                405 410 415
Phe Glu Asn Val Pro Phe His Ser Ser Tyr Ala His Ser Gln Ser Leu
                        420 425 430
Asp Arg Leu Met Asn Pro Leu Ile Asp Gln Tyr Leu Tyr Tyr Leu Ser
                435 440 445
Lys Thr Ile Asn Gly Ser Gly Gln Asn Gln Gln Thr Leu Lys Phe Ser
        450 455 460
Val Ala Gly Pro Ser Asn Met Ala Val Gln Gly Arg Asn Tyr Ile Pro
465 470 475 480
Gly Pro Ser Tyr Arg Gln Gln Arg Val Ser Thr Thr Val Thr Gln Asn
                                485 490 495
Asn Asn Ser Glu Phe Ala Trp Pro Gly Ala Ser Ser Trp Ala Leu Asn
                        500 505 510
Gly Arg Asn Ser Leu Met Asn Pro Gly Pro Ala Met Ala Ser His Lys
                515 520 525
Glu Gly Glu Asp Arg Phe Phe Pro Leu Ser Gly Ser Leu Ile Phe Gly
        530 535 540
Lys Gln Gly Thr Gly Arg Asp Asn Val Asp Ala Asp Lys Val Met Ile
545 550 555 560
Thr Asn Glu Glu Glu Ile Lys Thr Thr Asn Pro Val Ala Thr Glu Ser
                                565 570 575
Tyr Gly Gln Val Ala Thr Asn His Gln Ser Ala Gln Ala Ala Ala Lys
                        580 585 590
Gly Gly Gly Phe His Gly Ala Ser Ala Gln Ala Gln Thr Gly Trp Val
                595 600 605
Gln Asn Gln Gly Ile Leu Pro Gly Met Val Trp Gln Asp Arg Asp Val
        610 615 620
Tyr Leu Gln Gly Pro Ile Trp Ala Lys Ile Pro His Thr Asp Gly Asn
625 630 635 640
Phe His Pro Ser Pro Leu Met Gly Gly Phe Gly Met Lys His Pro Pro
                                645 650 655
Pro Gln Ile Leu Ile Lys Asn Thr Pro Val Pro Ala Asp Pro Pro Thr
                        660 665 670
Ala Phe Asn Lys Asp Lys Leu Asn Ser Phe Ile Thr Gln Tyr Ser Thr
                675 680 685
Gly Gln Val Ser Val Glu Ile Glu Trp Glu Leu Gln Lys Glu Asn Ser
        690 695 700
Lys Arg Trp Asn Pro Glu Ile Gln Tyr Thr Ser Asn Tyr Tyr Lys Ser
705 710 715 720
Asn Asn Val Glu Phe Ala Val Asn Thr Glu Gly Val Tyr Ser Glu Pro
                                725 730 735
Arg Pro Ile Gly Thr Arg Tyr Leu Thr Arg Asn Leu (SEQ ID NO: 12)
                        740 745
AAV1999 Capsid Amino Acid Sequence (VP2)
Thr Ala Pro Gly Lys Lys Arg
Pro Val Glu Gln Ser Pro Gln Glu Pro Asp Ser Ser Ala Gly Ile Gly
Lys Ser Gly Ala Gln Pro Ala Lys Lys Arg Leu Asn Phe Gly Gln Thr
Gly Asp Thr Glu Ser Val Pro Asp Pro Gln Pro Ile Gly Glu Pro Pro
Ala Ala Pro Ser Gly Val Gly Ser Leu Thr Met Ala Ser Gly Gly Gly
Ala Pro Val Ala Asp Asn Asn Glu Gly Ala Asp Gly Val Gly Ser Ser
Ser Gly Asn Trp His Cys Asp Ser Gln Trp Leu Gly Asp Arg Val Ile
Thr Thr Ser Thr Arg Thr Trp Ala Leu Pro Thr Tyr Asn Asn His Leu
Tyr Lys Gln Ile Ser Asn Ser Thr Ser Gly Gly Ser Ser Asn Asp Asn
Ala Tyr Phe Gly Tyr Ser Thr Pro Trp Gly Tyr Phe Asp Phe Asn Arg
Phe His Cys His Phe Ser Pro Arg Asp Trp Gln Arg Leu Ile Asn Asn
Asn Trp Gly Phe Arg Pro Lys Arg Leu Asn Phe Lys Leu Phe Asn Ile
Gln Val Lys Glu Val Thr Asp Asn Asn Gly Val Lys Thr Ile Ala Asn
Asn Leu Thr Ser Thr Val Gln Val Phe Thr Asp Ser Asp Tyr Gln Leu
Pro Tyr Val Leu Gly Ser Ala His Glu Gly Cys Leu Pro Pro Phe Pro
Ala Asp Val Phe Met Ile Pro Gln Tyr Gly Tyr Leu Thr Leu Asn Asp
Gly Ser Gln Ala Val Gly Arg Ser Ser Phe Tyr Cys Leu Glu Tyr Phe
Pro Ser Gln Met Leu Arg Thr Gly Asn Asn Phe Gln Phe Ser Tyr Glu
Phe Glu Asn Val Pro Phe His Ser Ser Tyr Ala His Ser Gln Ser Leu
Asp Arg Leu Met Asn Pro Leu Ile Asp Gln Tyr Leu Tyr Tyr Leu Ser
Lys Thr Ile Asn Gly Ser Gly Gln Asn Gln Gln Thr Leu Lys Phe Ser
Val Ala Gly Pro Ser Asn Met Ala Val Gln Gly Arg Asn Tyr Ile Pro
Gly Pro Ser Tyr Arg Gln Gln Arg Val Ser Thr Thr Val Thr Gln Asn
Asn Asn Ser Glu Phe Ala Trp Pro Gly Ala Ser Ser Trp Ala Leu Asn
Gly Arg Asn Ser Leu Met Asn Pro Gly Pro Ala Met Ala Ser His Lys
Glu Gly Glu Asp Arg Phe Phe Pro Leu Ser Gly Ser Leu Ile Phe Gly
Lys Gln Gly Thr Gly Arg Asp Asn Val Asp Ala Asp Lys Val Met Ile
Thr Asn Glu Glu Glu Ile Lys Thr Thr Asn Pro Val Ala Thr Glu Ser
Tyr Gly Gln Val Ala Thr Asn His Gln Ser Ala Gln Ala Ala Ala Lys
Gly Gly Gly Phe His Gly Ala Ser Ala gln Ala Gln Thr Gly Trp Val
Gln Asn Gln Gly Ile Leu Pro Gly met Val Trp Gln Asp Arg Asp Val
Tyr Leu Gln Gly Pro Ile Trp Ala Lys Ile Pro His Thr Asp Gly Asn
Phe His Pro Ser Pro Leu Met Gly Gly Phe Gly Met Lys His Pro Pro
Pro Gln Ile Leu Ile Lys Asn Thr Pro Val Pro Ala Asp Pro Pro Thr
Ala Phe Asn Lys Asp Lys Leu Asn Ser Phe Ile Thr Gln Tyr Ser Thr
Gly Gln Val Ser Val Glu Ile Glu Trp Glu Leu Gln Lys Glu Asn Ser
Lys Arg Trp Asn Pro Glu Ile Gln Tyr Thr Ser Asn Tyr Tyr Lys Ser
Asn Asn Val Glu Phe Ala Val Asn Thr Glu Gly Val Tyr Ser Glu Pro
Arg Pro Ile Gly Thr Arg Tyr Leu Thr Arg Asn Leu (SEQ ID NO: 13)
AAV1999 Capsid Amino Acid Sequence (VP3)
Met Ala Ser Gly Gly Gly
Ala Pro Val Ala Asp Asn Asn Glu Gly Ala Asp Gly Val Gly Ser Ser
Ser Gly Asn Trp His Cys Asp Ser Gln Trp Leu Gly Asp Arg Val Ile
Thr Thr Ser Thr Arg Thr Trp Ala Leu Pro Thr Tyr Asn Asn His Leu
Tyr Lys Gln Ile Ser Asn Ser Thr Ser Gly Gly Ser Ser Asn Asp Asn
Ala Tyr Phe Gly Tyr Ser Thr Pro Trp Gly Tyr Phe Asp Phe Asn Arg
Phe His Cys His Phe Ser Pro Arg Asp Trp Gln Arg Leu Ile Asn Asn
Asn Trp Gly Phe Arg Pro Lys Arg Leu Asn Phe Lys Leu Phe Asn Ile
Gln Val Lys Glu Val Thr Asp Asn Asn Gly Val Lys Thr Ile Ala Asn
Asn Leu Thr Ser Thr Val Gln Val Phe Thr Asp Ser Asp Tyr Gln Leu
Pro Tyr Val Leu Gly Ser Ala His Glu Gly Cys Leu Pro Pro Phe Pro
Ala Asp Val Phe Met Ile Pro Gln Tyr Gly Tyr Leu Thr Leu Asn Asp
Gly Ser Gln Ala Val Gly Arg Ser Ser Phe Tyr Cys Leu Glu Thr Phe
Pro Ser Gln Met Leu Arg Thr Gly Asn Asn Phe Gln Phe Ser Tyr Glu
Phe Glu Asn Val Pro Phe His Ser Ser Tyr Ala His Ser Gln Ser Leu
Asp Arg Leu Met Asn Pro Leu Ile Asp Gln Tyr Leu Tyr Tyr Leu Ser
Lys Thr Ile Asn Gly Ser Gly Gln Asn Gln Gln Thr Leu Lys Phe Ser
Val Ala Gly Pro Ser Asn Met Ala Val Gln Gly Arg Asn Tyr Ile Pro
Gly Pro Ser Tyr Arg Gln Gln Arg Val Ser Thr Thr Val Thr Gln Asn
Asn Asn Ser Glu Phe Ala Trp Pro gly Ala Ser Ser Trp Ala Leu Asn
Gly Arg Asn Ser Leu Met Asn Pro Gly Pro Ala Met Ala Ser His Lys
Glu Gly Glu Asp Arg Phe Phe Pro Leu Ser Gly Ser Leu Ile Phe Gly
Lys Gln Gly Thr Gly Arg Asp Asn Val Asp Ala Asp Lys Val Met Ile
Thr Asn Glu Glu Glu Ile Lys Thr Thr Asn Pro Val Ala Thr Glu Ser
Tyr Gly Gln Val Ala Thr Asn His Gln Ser Ala Gln Ala Ala Ala Lys
Gly Gly Gly Phe His Gly Ala Ser Ala Gln Ala Gln Thr Gly Trp Val
Gln Asn Gln Gly Ile Leu Pro Gly Met Val Trp Gln Asp Arg Asp Val
Tyr Leu Gln Gly Pro Ile Trp Ala Lys Ile Pro His Thr Asp Gly Asn
Phe His Pro Ser Pro Leu Met Gly Gly Phe Gly Met Lys His Pro Pro
Pro Gln Ile Leu Ile Lys Asn Thr Pro Val Pro Ala Asp Pro Pro Thr
Ala Phe Asn Lys Asp Lys Leu Asn Ser Phe Ile Thr Gln Tyr Ser Thr
Gly Gln Val Ser Val Glu Ile Glu Trp Glu Leu Gln Lys Glu Asn Ser
Lys Arg Trp Asn Pro Glu Ile Gln Tyr Thr Ser Asn Tyr Tyr Lys Ser
Asn Asn Val Glu Phe Ala Val Asn Thr Glu Gly Val Tyr Ser Glu Pro
Arg Pro Ile Gly Thr Arg Tyr Leu Thr Arg Asn Leu (SEQ ID NO: 14)
Full DNA sequence of vector; coding sequence for ARSA is underlined
TTGGCCACTCCCTCTCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAACTCCATCACTAG
GGGTTCCTTACGTACAATTGGGATCCCGGACCGTCGACATTGATTATTGACTAGTTATTAATAGTAATCA
ATTACGGGGTCATTAGTTCATAGCCCATATATGGAGTTCCGCGTTACATAACTTACGGTAAATGGCCCGC
CTGGCTGACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGCTATTACCATGGTCGA
GGTGAGCCCCACGTTCTGCTTCACTCTCCCCATCTCCCCCCCCTCCCCACCCCCAATTTTGTATTTATTTATT
TTTTAATTATTTTGTGCAGCGATGGGGGCGGGGGGGGGGGGGGGGCGCGCGCCAGGCGGGGGGGG
CGGGGCGAGGGGCGGGGGGGGCGAGGCGGAGAGGTGCGGCGGCAGCCAATCAGAGCGGCGCGCT
CCGAAAGTTTCCTTTTATGGCGAGGCGGCGGCGGCGGCGGCCCTATAAAAAGCGAAGCGCGCGGCGG
GCGGGAGTCGCTGCGCGCTGCCTTCGCCCCGTGCCCCGCTCCGCCGCCGCCTCGCGCCGCCCGCCCCGG
CTCTGACTGACCGCGTTACTCCCACAGGTGAGCGGGCGGGACGGCCCTTCTCCTCCGGGCTGTAATTAG
CGCTTGGTTTAATGACGGCTTGTTTCTTTTCTGTGGCTGCGTGAAAGCCTTGAGGGGCTCCGGGAGGGC
CCTTTGTGCGGGGGGAGCGGCTCGGGGGGTGCGTGCGTGTGTGTGTGCGTGGGGAGCGCCGCGTGCG
GCTCCGCGCTGCCCGGCGGCTGTGAGCGCTGCGGGCGCGGCGCGGGGCTTTGTGCGCTCCGCAGTGTG
CGCGAGGGGAGCGCGGCCGGGGGCGGTGCCCCGCGGTGCGGGGGGGGCTGCGAGGGGAACAAAGG
CTGCGTGCGGGGTGTGTGCGTGGGGGGGTGAGCAGGGGGTGTGGGCGCGTCGGTCGGGCTGCAACCC
CCCCTGCACCCCCCTCCCCGAGTTGCTGAGCACGGCCCGGCTTCGGGTGCGGGGCTCCGTACGGGGCGT
GGCGCGGGGCTCGCCGTGCCGGGGGGGGGGGGCGGCAGGTGGGGGTGCCGGGCGGGGGGGGGCC
GCCTCGGGCCGGGGAGGGCTCGGGGGAGGGGCGCGGCGGCCCCCGGAGCGCCGGCGGCTGTCGAGG
CGCGGCGAGCCGCAGCCATTGCCTTTTATGGTAATCGTGCGAGAGGGCGCAGGGACTTCCTTTGTCCCA
AATCTGTGCGGAGCCGAAATCTGGGAGGCGCCGCCGCACCCCCTCTAGCGGGCGCGGGGCGAAGCGG
TGCGGCGCCGGCAGGAAGGAAATGGGGGGGGAGGGCCTTCGTGCGTCGCCGCGCCGCCGTCCCCTTCT
CCCTCTCCAGCCTCGGGGCTGTCCGCGGGGGGACGGCTGCCTTCGGGGGGGACGGGGCAGGGCGGGG
TTCGGCTTCTGGCGTGTGACCGGCGGCTCTAGAGCCTCTGCTAACCATGTTCATGCCTTCTTCTTTTTCCT
ACAGCTCCTGGGCAACGTGCTGGTTATTGTGCTGTCTCATCATTTTGGCAAAGAATTCTACGTACCACCA
TGAGCATGGGAGCCCCTAGATCTCTGCTGCTGGCTCTTGCTGCTGGACTGGCTGTGGCCAGACCTCCTA
ACATCGTGCTGATCTTCGCCGACGATCTCGGCTATGGCGATCTGGGCTGTTACGGACACCCTAGCAGCA
CCACACCTAACCTGGATCAACTGGCTGCCGGCGGACTGAGATTCACCGATTTCTACGTGCCCGTGTCTCT
GTGCACACCTAGTAGAGCTGCTCTGCTGACAGGCAGACTGCCAGTGCGGATGGGAATGTATCCTGGCGT
GCTGGTTCCTAGCAGTAGAGGCGGACTGCCTCTGGAAGAAGTGACAGTTGCTGAAGTGCTGGCCGCCA
GAGGCTATCTGACTGGAATGGCCGGAAAATGGCACCTCGGAGTTGGACCTGAAGGCGCTTTTCTGCCTC
CTCACCAGGGCTTCCACAGATTTCTGGGCATCCCTTACAGCCACGATCAGGGCCCTTGCCAGAACCTGAC
CTGCTTTCCTCCTGCCACACCTTGTGATGGCGGCTGTGATCAGGGACTCGTGCCTATTCCTCTGCTGGCC
AATCTGAGCGTGGAAGCTCAACCTCCTTGGCTGCCTGGCCTGGAAGCCAGATATATGGCCTTCGCTCAC
GACCTGATGGCCGACGCTCAGAGACAGGACAGACCATTCTTCCTGTACTACGCCAGCCACCACACACAC
TACCCTCAGTTCTCTGGCCAGTCCTTCGCCGAGAGATCTGGCAGAGGCCCTTTTGGCGATAGCCTGATGG
AACTGGATGCCGCCGTGGGAACACTGATGACAGCCATTGGAGATCTGGGCCTGCTGGAAGAGACACTG
GTCATCTTCACCGCCGACAACGGCCCCGAGACAATGAGAATGAGCAGAGGCGGCTGTAGCGGCCTGCT
GAGATGTGGCAAGGGAACAACATACGAAGGCGGCGTCAGAGAGCCTGCTCTGGCTTTTTGGCCTGGAC
ATATTGCCCCTGGCGTGACACACGAACTGGCCTCTTCTCTGGATCTGCTGCCTACACTGGCTGCTTTGGC
TGGCGCTCCTCTGCCTAATGTGACCCTGGATGGCTTCGATCTGTCTCCACTGCTGCTCGGAACAGGCAAG
AGCCCTAGACAGAGCCTGTTCTTCTACCCTAGCTACCCCGATGAAGTGCGGGGAGTGTTTGCCGTGCGG
ACAGGCAAGTACAAGGCCCACTTTTTTACCCAAGGCAGCGCCCACAGCGATACCACAGCTGATCCTGCTT
GTCACGCCTCTAGCAGCCTGACAGCTCATGAACCACCTCTGCTGTACGACCTGTCTAAGGACCCCGGCGA
GAACTATAATCTGCTTGGCGGAGTTGCCGGCGCTACACCTGAAGTTCTGCAGGCTCTGAAACAGCTCCA
GCTGCTGAAAGCCCAGCTGGACGCTGCTGTGACATTTGGACCTTCTCAGGTGGCAAGAGGCGAGGACC
CTGCTCTGCAGATTTGTTGTCACCCTGGCTGTACCCCTAGACCTGCCTGCTGTCACTGTCCTGATCCTCAC
GCTTGAGATTAATCAACCTCTGGATTACAAAATTTGTGAAAGATTGACTGGTATTCTTAACTATGTTGCTC
CTTTTACGCTATGTGGATACGCTGCTTTAATGCCTTTGTATCATGCTATTGCTTCCCGTATGGCTTTCATTT
TCTCCTCCTTGTATAAATCCTGGTTGCTGTCTCTTTATGAGGAGTTGTGGCCCGTTGTCAGGCAACGTGG
CGTGGTGTGCACTGTGTTTGCTGACGCAACCCCCACTGGTTGGGGCATTGCCACCACCTGTCAGCTCCTT
TCCGGGACTTTCGCTTTCCCCCTCCCTATTGCCACGGCGGAACTCATCGCCGCCTGCCTTGCCCGCTGCTG
GACAGGGGCTCGGCTGTTGGGCACTGACAATTCCGTGGTGTTGTCGGGGAAATCATCGTCCTTTCCTTG
GCTGCTCGCCTGTGTTGCCACCTGGATTCTGCGCGGGACGTCCTTCTGCTACGTCCCTTCGGCCCTCAATC
CAGCGGACCTTCCTTCCCGCGGCCTGCTGCCGGCTCTGCGGCCTCTTCCGCGTCTTCGCCTTCGCCCTCAG
ACGAGTCGGATCTCCCTTTGGGCCGCCTCCCCGCCTGATCCTGTGCCTTCTAGTTGCCAGCCATCTGTTGT
TTGCCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAG
GGGGAGGATTGGGAAGACAATAGCAGGCATGCTCGAAATTAGGAACCCCTAGTGATGGAGTTGGCCAC
TCCCTCTCTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAA (SEQ ID NO: 16)
Underlined, codon Optimized ARSA DNA sequence (Human)
```

1. A recombinant adeno-associated virus (rAAV) particle comprising (1) a rAAV vector comprising an expression cassette for expressing an arylsulfatase A (ARSA) polypeptide, wherein the expression cassette comprises a gene encoding the ARSA polypeptide operably linked to a promoter and optionally an enhancer, and (2) a modified AAV9 capsid protein comprising a targeting peptide that has SEQ ID NO: 10.

2. The rAAV particle of claim 1, wherein the ARSA polypeptide comprises SEQ ID NO: 1.

3. The rAAV particle of claim 2, wherein the targeting peptide is flanked by linker sequences on its N-terminal end and the C-terminal end.

4. The rAAV particle of claim 3, wherein the combined targeting peptide and linker sequences comprise SEQ ID NO: 11.

5. The rAAV particle of claim 4, wherein modified capsid protein has a sequence that is at least 98.5% identical to SEQ ID NO: 12.

6. The rAAV particle of claim 5, wherein the modified capsid protein comprises a sequence comprising SEQ ID NO: 12.

7. The rAAV particle of claim 6, wherein the expression cassette comprises a codon optimized ARSA gene of SEQ ID NO: 2.

8. The rAAV particle of claim 7, wherein the rAAV vector comprises a 5′ AAV2 ITR of SEQ ID NO: 4 and a 3′ AAV2 ITR of SEQ ID NO: 5.

9. The rAAV particle of claim 8, wherein the expression cassette comprises a CMV enhancer element comprising SEQ ID NO: 6.

10. The rAAV particle of claim 9, wherein the expression cassette comprises a chicken b-actin promoter comprising SEQ ID NO: 7.

11. The rAAV particle of claim 10, wherein the rAAV vector further comprises a WPRE element.

12. The rAAV particle of claim 11, wherein the WPRE element comprises a sequence of SEQ ID NO:8.

13. The rAAV particle of claim 12, wherein the rAAV vector comprises a sequence of SEQ ID NO:1615.

14. A method of treating metachromatic leukodystrophy (MLD) in a human patient in need thereof, comprising administering to the cerebrospinal fluid (CSF) of the patient a composition comprising an effective amount of recombinant adeno-associated virus (rAAV) viral particle of claim 6.

15. The method of claim 14, wherein the composition is administered directly to the CSF of the patient via intracerebroventricular (ICV) administration.

16. The method of claim 14, wherein the composition is administered directly to the CSF of the patient via direct cisterna magna (dCM) administration.

17. The method of claim 14, wherein the composition is administered directly to the CSF of the patient with an intrathecal microcatheter (IT-CM).

18. The method of claim 14, wherein the composition is administered only once over the lifetime of the patient.

19. The method of claim 14, wherein the composition is administered only once yearly to the patient.

20. The method of claim 14, wherein said administering increases ARSA activity by at least 5% in the patient.

21-24. (canceled)

25. A method of increasing expression and/or activity of ARSA in an individual in need thereof, comprising administering to the cerebrospinal fluid (CSF) of the patient a composition comprising an effective amount of recombinant adeno-associated virus (rAAV) viral particle of claim 1.

26-49. (canceled)


SEQUENCE LISTING (16 sequences)

SEQ ID NO: 1 - protein, 507 aa, Homo sapiens
MGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 2 - DNA, 1530 bp, Homo sapiens
  source 1..1530: mol_type = unassigned DNA
atgagcatgggagcccctagatctctgctgctggctcttgctgctggactggctgtggccagacctcctaacatcgtgctgatcttcgccgacgatctcggctatggcgatctgggctgttacggacaccctagcagcaccacacctaacctggatcaactggctgccggcggactgagattcaccgatttctacgtgcccgtgtctctgtgcacacctagtagagctgctctgctgacaggcagactgccagtgcggatgggaatgtatcctggcgtgctggttcctagcagtagaggcggactgcctctggaagaagtgacagttgctgaagtgctggccgccagaggctatctgactggaatggccggaaaatggcacctcggagttggacctgaaggcgcttttctgcctcctcaccagggcttccacagatttctgggcatcccttacagccacgatcagggcccttgccagaacctgacctgctttcctcctgccacaccttgtgatggcggctgtgatcagggactcgtgcctattcctctgctggccaatctgagcgtggaagctcaacctccttggctgcctggcctggaagccagatatatggccttcgctcacgacctgatggccgacgctcagagacaggacagaccattcttcctgtactacgccagccaccacacacactaccctcagttctctggccagtccttcgccgagagatctggcagaggcccttttggcgatagcctgatggaactggatgccgccgtgggaacactgatgacagccattggagatctgggcctgctggaagagacactggtcatcttcaccgccgacaacggccccgagacaatgagaatgagcagaggcggctgtagcggcctgctgagatgtggcaagggaacaacatacgaaggcggcgtcagagagcctgctctggctttttggcctggacatattgcccctggcgtgacacacgaactggcctcttctctggatctgctgcctacactggctgctttggctggcgctcctctgcctaatgtgaccctggatggcttcgatctgtctccactgctgctcggaacaggcaagagccctagacagagcctgttcttctaccctagctaccccgatgaagtgcggggagtgtttgccgtgcggacaggcaagtacaaggcccacttttttacccaaggcagcgcccacagcgataccacagctgatcctgcttgtcacgcctctagcagcctgacagctcatgaaccacctctgctgtacgacctgtctaaggaccccggcgagaactataatctgcttggcggagttgccggcgctacacctgaagttctgcaggctctgaaacagctccagctgctgaaagcccagctggacgctgctgtgacatttggaccttctcaggtggcaagaggcgaggaccctgctctgcagatttgttgtcaccctggctgtacccctagacctgcctgctgtcactgtcctgatcctcacgcttga

SEQ ID NO: 3 - DNA, 10788 bp, synthetic construct
  source 1..10788: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacaattgggatcccggaccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctacgtaccaccatgagcatgggagcccctagatctctgctgctggctcttgctgctggactggctgtggccagacctcctaacatcgtgctgatcttcgccgacgatctcggctatggcgatctgggctgttacggacaccctagcagcaccacacctaacctggatcaactggctgccggcggactgagattcaccgatttctacgtgcccgtgtctctgtgcacacctagtagagctgctctgctgacaggcagactgccagtgcggatgggaatgtatcctggcgtgctggttcctagcagtagaggcggactgcctctggaagaagtgacagttgctgaagtgctggccgccagaggctatctgactggaatggccggaaaatggcacctcggagttggacctgaaggcgcttttctgcctcctcaccagggcttccacagatttctgggcatcccttacagccacgatcagggcccttgccagaacctgacctgctttcctcctgccacaccttgtgatggcggctgtgatcagggactcgtgcctattcctctgctggccaatctgagcgtggaagctcaacctccttggctgcctggcctggaagccagatatatggccttcgctcacgacctgatggccgacgctcagagacaggacagaccattcttcctgtactacgccagccaccacacacactaccctcagttctctggccagtccttcgccgagagatctggcagaggcccttttggcgatagcctgatggaactggatgccgccgtgggaacactgatgacagccattggagatctgggcctgctggaagagacactggtcatcttcaccgccgacaacggccccgagacaatgagaatgagcagaggcggctgtagcggcctgctgagatgtggcaagggaacaacatacgaaggcggcgtcagagagcctgctctggctttttggcctggacatattgcccctggcgtgacacacgaactggcctcttctctggatctgctgcctacactggctgctttggctggcgctcctctgcctaatgtgaccctggatggcttcgatctgtctccactgctgctcggaacaggcaagagccctagacagagcctgttcttctaccctagctaccccgatgaagtgcggggagtgtttgccgtgcggacaggcaagtacaaggcccacttttttacccaaggcagcgcccacagcgataccacagctgatcctgcttgtcacgcctctagcagcctgacagctcatgaaccacctctgctgtacgacctgtctaaggaccccggcgagaactataatctgcttggcggagttgccggcgctacacctgaagttctgcaggctctgaaacagctccagctgctgaaagcccagctggacgctgctgtgacatttggaccttctcaggtggcaagaggcgaggaccctgctctgcagatttgttgtcaccctggctgtacccctagacctgcctgctgtcactgtcctgatcctcacgcttgagattaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcgaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaatcgcgacggccgacgtctttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcattgtaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaaggctaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaagctaactgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgaaagctctggaggggcctagaagtggtaaatgaatgaaaacgtggttactctcccgatctgcctttcccaaatatggccattcttggctgaatcagaaatcaaaggacaggttattaattactagctctaagttacttaccatttgctgagacagttcagaaatctgactgcatctcctcagggatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctatattccattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttgacgtcattaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgccaacgcgt

SEQ ID NO: 4 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 5 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 6 - DNA, 381 bp, synthetic construct
  source 1..381: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 7 - DNA, 323 bp, synthetic construct
  source 1..323: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgcc

SEQ ID NO: 8 - DNA, 594 bp, synthetic construct
  source 1..594: mol_type = other DNA
ttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 9 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 7 aa, synthetic construct
KGGGFHG

SEQ ID NO: 11 - protein, 12 aa, synthetic construct
AAAKGGGFHGAS

SEQ ID NO: 12 - protein, 748 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAKGGGFHGASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 611 aa, synthetic construct
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAKGGGFHGASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 546 aa, synthetic construct
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAKGGGFHGASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 78 bp, synthetic construct
  source 1..78: mol_type = other DNA
cactccctctctgcgcgctcgctcgctcactgaggccgggcgaccaaaggtcgcccacgcccgggctttgcccgggcg

SEQ ID NO: 16 - DNA, 4397 bp, synthetic construct
  source 1..4397: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacaattgggatcccggaccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctacgtaccaccatgagcatgggagcccctagatctctgctgctggctcttgctgctggactggctgtggccagacctcctaacatcgtgctgatcttcgccgacgatctcggctatggcgatctgggctgttacggacaccctagcagcaccacacctaacctggatcaactggctgccggcggactgagattcaccgatttctacgtgcccgtgtctctgtgcacacctagtagagctgctctgctgacaggcagactgccagtgcggatgggaatgtatcctggcgtgctggttcctagcagtagaggcggactgcctctggaagaagtgacagttgctgaagtgctggccgccagaggctatctgactggaatggccggaaaatggcacctcggagttggacctgaaggcgcttttctgcctcctcaccagggcttccacagatttctgggcatcccttacagccacgatcagggcccttgccagaacctgacctgctttcctcctgccacaccttgtgatggcggctgtgatcagggactcgtgcctattcctctgctggccaatctgagcgtggaagctcaacctccttggctgcctggcctggaagccagatatatggccttcgctcacgacctgatggccgacgctcagagacaggacagaccattcttcctgtactacgccagccaccacacacactaccctcagttctctggccagtccttcgccgagagatctggcagaggcccttttggcgatagcctgatggaactggatgccgccgtgggaacactgatgacagccattggagatctgggcctgctggaagagacactggtcatcttcaccgccgacaacggccccgagacaatgagaatgagcagaggcggctgtagcggcctgctgagatgtggcaagggaacaacatacgaaggcggcgtcagagagcctgctctggctttttggcctggacatattgcccctggcgtgacacacgaactggcctcttctctggatctgctgcctacactggctgctttggctggcgctcctctgcctaatgtgaccctggatggcttcgatctgtctccactgctgctcggaacaggcaagagccctagacagagcctgttcttctaccctagctaccccgatgaagtgcggggagtgtttgccgtgcggacaggcaagtacaaggcccacttttttacccaaggcagcgcccacagcgataccacagctgatcctgcttgtcacgcctctagcagcctgacagctcatgaaccacctctgctgtacgacctgtctaaggaccccggcgagaactataatctgcttggcggagttgccggcgctacacctgaagttctgcaggctctgaaacagctccagctgctgaaagcccagctggacgctgctgtgacatttggaccttctcaggtggcaagaggcgaggaccctgctctgcagatttgttgtcaccctggctgtacccctagacctgcctgctgtcactgtcctgatcctcacgcttgagattaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcgaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa